(19) United States
(12) Patent Application Publication
(10) Pub. No.: US 2007/0219214 A1
(43) Pub. Date: Sep. 20, 2007

(54) Dual NK2/NK3-antagonists, pharmaceutical compositions comprising them, and processes for their preparation

(75) Inventors: Daniel Jasserand, Hannover (DE); Matthias Mentzel, Hannover (DE); Janis Jaunzems, Burgdorf (DE); Werner Benson, Seelze (DE); Uwe Schon, Burgdorf (DE); Pieter Smid, Voorhout (NL); Ulf Preuschoff, Isernhagen (DE); Michael Firnges, Barsinghausen (DE); Holger Sann, Hannover (DE); Reinhard Brueckner, Hannover (DE); Martinus Adolfs, Almere (NL); Amos Attali, Amsterdam (NL); Dania Reiche, Adelheidsdorf (DE); Andrew Christopher McCreary, Almere (NL)

Correspondence Address: CROWELL & MORING LLP;INTELLECTUAL PROPERTY GROUP, P.O. BOX 14300, WASHINGTON, DC, 20044-4300, US

(73) Assignee: Solvay pharmaceuticals GmbH, Hannover (DE)

(21) Appl. No.: 11/700,788

(22) Filed: Feb. 1, 2007

Related U.S. Application Data
(60) Provisional application No. 60/764,073, filed on Feb. 1, 2006.

(51) Int. Cl. A61K 31/495; A61K 31/445; C07D 211/26; C07D 241/04

(52) U.S. Cl. 514/252.12; 514/317; 544/392; 544/399; 546/229

(57) ABSTRACT

Dual NK2/NK3-antagonists corresponding to formula I: 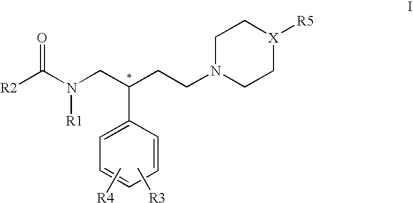 and physiologically compatible salts of such compounds in which X and R1 to R5 have specific defined meanings, pharmaceutical compositions containing such compounds, methods of using such compounds to treat or inhibit disorders mediated by tachykinin receptors, and a process for preparing such compounds.

### CROSS REFERENCE TO RELATED APPLICATION

This application claims priority from co-pending U.S. provisional patent application No. 60/764,073, filed Feb. 1, 2006.

### BACKGROUND OF THE INVENTION

The present invention relates to novel dual NK2/NK3-antagonists and also to pharmaceutical compositions comprising these compounds. Furthermore, the invention relates to processes for the preparation of the novel dual NK2/NK3-antagonists.

Neurokinins (NKs) also known as tachykinins, include the naturally-occurring neuropeptides substance P, neurokinin A and neurokinin B. The tachykinins act as agonists of receptors occurring in larger mammals and humans, such as the neurokinin-1 receptor, the NK-2 receptor and the NK-3 receptor. Artificially prepared compounds which are antagonistic to tachykinin receptors are usually classified according to their relative ability to bind to one or more of the aforementioned three receptor subtypes. In the physiological process the tachykinins play e.g. an important part in the transmission of pain, emesis, neurogenic inflammation, bladder inflammation, inflammatory joint diseases or asthmatic complaints.

A review article about NK receptor antagonists was recently published by Gerspacher (Progress in Medical Chemistry, 2005, Vol. 43, 49 to 103) giving an overview about recent developments on selective (NK1- or NK2- or NK3-receptor antagonists) and on combined (NK1/NK2-; NK1/NK2/NK3-; and NK2/NK3-) receptor antagonists.

The class of combined NK2/NK3-receptor antagonists appears to be limited by two approaches from GSK and Sanofi. GSK preferred a stepwise modification of the structure of the NK3 selective antagonist talnetant, by the introduction of a variety of substituents at the meta-position of the quinoline moiety of the molecule. A highly effective compound is the following:

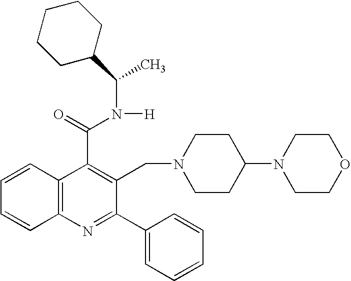

Sanofi-Syntheselabo describes in WO 2002/094821 (published Nov. 28, 2002) a series of piperidine-carboxamide derivatives having the following cyclic, non-linear general structure: 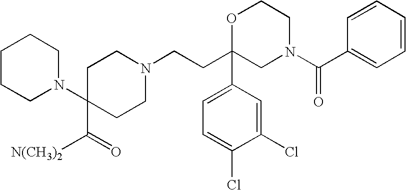
Sanofi-Syntheselabo reports a potent NK2-receptor affinity with a Ki value of 0.04 nM and a potent NK3-receptor affinity with a Ki value of 0.04 nM.

However, compounds with a linear core having either a selective NK3-receptor affinity or a combined NK2-/NK3-receptor affinity have not been reported so far.

### SUMMARY OF THE INVENTION

It was therefore an object of the present invention to provide novel active compounds with a linear core having properties antagonistic to NK2- and/or NK3 tachykinin receptors.

Another object of the invention was to provide NK2- and/or NK3-tachykinin receptor antagonists with a linear core.

A further aspect of the invention was to provide compounds suitable for treating or inhibiting a variety of disorders mediated by the NK2- and/or NK3-tachykinin receptor, as well as a method for treating or inhibiting such disorders utilizing such compounds.

Surprisingly, it has now been found that a group of novel linear core-compounds is distinguished by properties antagonistic to tachykinin receptors, in particular to NK2 and/or NK3-receptors. Accordingly, the group of compounds according to the invention appears particularly suitable for the treatment of peripheral disorders in which tachykinins, in particular neurokinin A and/or neurokinin B, participate as transfer agents, for example for the treatment and/or inhibition of any pathology where either neurokinin A and/or NK2-receptors, or neurokinin B and/or NK3-receptors, or both neurokinin A and neurokinin B and/or NK2 and NK3-receptors are involved.

In more detail, the compounds of the present invention appear particularly suitable for the treatment and/or inhibition of pathologies of the respiratory, gastrointestinal, urinary, immune and cardiovascular systems and of the central nervous system as well as pain, migraine, inflammation, nausea and vomiting, and skin diseases.

In even more detail, the compounds of the present invention appear particularly suitable for the treatment and/or inhibition of pathologies of respiratory diseases, in particular asthma, chronic obstructive pulmonary disease, chronic obstructive bronchitis, bronchitis, cough, and rhinitis; skin diseases, in particular inflammatory skin reactions, allergic skin reactions, and psoriasis; arthropathy diseases, in particular arthritis, vasculitides and systemic lupus erythematosus; functional or inflammatory disorders in the gastrointestinal tract, in particular pseudomembranous colitis, gastritis, acute and chronic pancreatitis, ulcerative colitis, Crohn's disease and diarrhea bladder diseases such as cystitis and interstitial cystitis; cardiovascular diseases such as hypertension, treatment of cancer especially melanomas, gliomas, small-cell and large-cell lung cancers, diseases of the immune system, bipolar disorders; migraine; pain, anxiety, depression, cognitive disorders, stress-related somatic disorders, psychosis, in particular schizophrenia, mania, schizoaffective disorder and panic disorders.

The present invention thus relates to compounds of formula I: 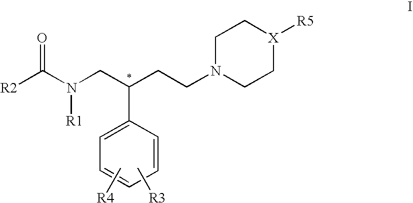
wherein
R1 is selected from the group consisting of alkyl and cycloalkyl;
R2 is selected from the group consisting of alkyl, cycloalkyl, aryl, alkylenearyl, alkenylenearyl, heteroaryl, and heterocyclic ring;
R3 and R4 are independently selected from the group consisting of hydrogen, halogen, hydroxyl, cyano, and carboxyalkyl;
X is selected from the group consisting of CR6 and nitrogen;
R5 is selected from the group consisting of alkyl optionally substituted with (CO)ₘNR9R10, cycloalkyl optionally substituted with (CO)ₘNR9R10, and NR7R8;
R6 is selected from the group consisting of hydrogen, alkyl, cycloalkyl, and (CO)ₘNR9R10;
R7 and R8 are independently selected from the group consisting of alkyl, cycloalkyl, aryl, alkylenearyl, alkyleneoxyalkyl, COalkyl, COaryl, or wherein R7 and R8 form together a 5- to 7-membered ring optionally containing an additional heteroatoms wherein such ring may be substituted by CONR9R10, and wherein in a 6-membered ring, none of the ring atoms is replaced by carbonyl;
R9 and R10 are independently selected from the group consisting of hydrogen, alkyl, cycloalkyl, aryl, alkylenearyl, alkyleneoxyalkyl, or wherein R9 and R10 form together a 5- to 7-membered ring optionally containing an additional heteroatom;
m is 0 or 1;
   and physiologically compatible salts, especially acid addition salts, of compounds of Formula I. Furthermore, the invention also relates to pharmaceutical compositions comprising the compounds of Formula I.

In another aspect, the invention also relates to processes for the preparation of the compounds of Formula I.

Where in the compounds of Formula I or in other compounds described within the scope of the present invention substituents are or contain alkyl and/or alkylene, these may each be straight-chain or branched and contain from 1 to 20 carbon atoms, preferably from 1 to 10 carbon atoms, more preferably from 1 to 7 carbon atoms and even more preferably from 1 to 4 carbon atoms. The most preferred straight-chain alkyl and/or alkylene group is methyl and/or methylene, respectively. The most preferred branched alkyl and/or alkylene group is isopropyl and/or isopropylene, respectively.

Where in the compounds of Formula I or in other compounds described within the scope of the present invention substituents are or contain cycloalkyl and/or cycloalkylene, these may possess from 1 to 20 carbon atoms, preferably from 1 to 10 carbon atoms, more preferably from 1 to 7 carbon atoms and even more preferably from 1 to 4 carbon atoms. The most preferred cycloalkyl and/or cycloalkylene groups are cyclopentyl and cyclohexyl and/or cyclopentylene and cyclohexylene, respectively.

Where in the compounds of Formula I or in other compounds described within the scope of the present invention substituents are or contain alkenyl and/or alkenylene, these may each be straight-chain or branched and possess from 2 to 20 carbon atoms, preferably from 2 to 10 carbon atoms, more preferably from 2 to 7 carbon atoms and even more preferably from 2 to 4 carbon atoms. The most preferred alkenyl and/or alkenylene group is ethenyl and/or ethenylene, respectively.

Where substituents in compounds of Formula I are or contain halogen, fluorine, chlorine or bromine are suitable. Chlorine is preferred. Where substituents in compounds of Formula I are or contain carboxyalkyl, —OC(O)alkyl or C(O)Oalkyl are suitable, OC(O)alkyl is preferred.

Where substituents in compounds of Formula I are or contain aryl and/or arylene, monocyclic, bicyclic, tricyclic and tetracyclic aromatic ring systems are suitable. Phenyl is preferred.

Where substituents in compounds of Formula I are or contain heteroaryl and/or heteroarylene, monocyclic, bicyclic, tricyclic and tetracyclic aromatic ring systems containing at least one heteroatom such as nitrogen are suitable.

Where substituents in compounds of Formula I are or contain heterocyclic rings, monocyclic, bicyclic, tricyclic and tetracyclic non-aromatic ring systems containing at least one, if not two or three or even four heteroatoms such as nitrogen and/or sulfur and/or oxygen are suitable. A monocyclic ring system is preferred. Nitrogen and/or oxygen are preferred as heteroatoms.

Where R7 and R8 and/or R9 and R10 form together a 5- to 7-membered ring and wherein in a particular embodiment, each of these rings independently optionally contain an additional heteroatom, such heteroatom may be selected from nitrogen, oxygen and sulfur, preferably oxygen.

Where in the compounds of Formula I or in other compounds described within the scope of the present invention are or contain alkyl and/or alkylene, alkyl and/or alkylene, aryl and/or arylene, heteroaryl and/or heteroarylene, heterocyclic rings, all these substituents may be further substituted by any of alkyl, alkenyl, hydroxyl, SH, carbonylalkyl, carboxyalkyl, carbonylaryl, carboxyaryl, carbonylheteroaryl, carboxyheteroyaryl, carbonylheterocyclic ring, carboxyheterocyclic ring, halogen, cyano, oxyalkyl, oxyalkenyl, aryl, heteroaryl, NO₂, SO₂R11, and SO₃R11.

In one preferred embodiment of the present invention, in compounds of Formula I, R1 is methyl.

In another preferred embodiment of the present invention, R3 and R4 are independently selected from the group consisting of hydrogen, fluoro, chloro, preferably hydrogen or chloro.

In third preferred embodiment of the present invention, in compounds of Formula I X is CR6, R5 is NR7R8, and R6 is (CO)ₘNR9R10 with m=1.

In yet another preferred embodiment of the present invention, in compounds X is N, R5 is cycloalkyl substituted with (CO)ₘNR9R10 and m=1.

In a fourth preferred embodiment of the present invention, in compounds of Formula I, R7 and R8 form together a 6-membered ring or R7 and R8 form together a 6-membered ring substituted by CONR9R10.

In a fifth preferred embodiment of the present invention, in compounds of Formula I R9 and R10 are both methyl, or R9 and R10 form together a 6-membered ring, or R9 and R10 form together a 5-membered ring substituted by carbonyl.

In another preferred embodiment of the present invention, in compounds of Formula I R2 is selected from the group consisting of C₁ to C₂₀ alkyl; C₃ to C₂₀ cycloalkyl; C₂ to C₂₀ alkenyl; 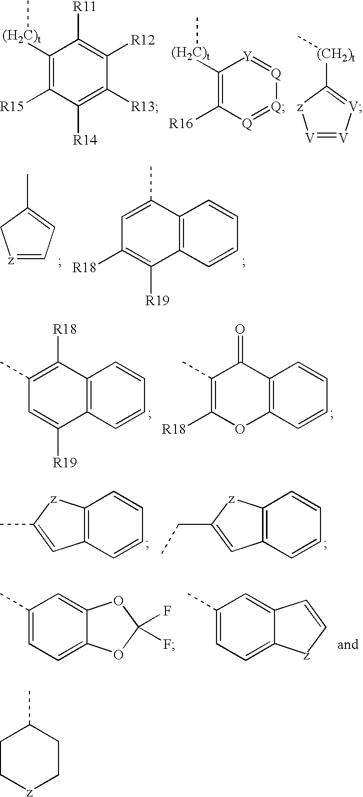
wherein
each of R11 to R16 are independently selected from the group consisting of hydrogen, fluoro, chloro, bromo, hydroxyl, alkoxy, cyano, N(H)C(O)Oalkyl, aminoalkyl, dialkylamino, OCF₃, CF₃, carboxyalkyl, S(O)₂NH₂, phenyl, alkyl, and cycloalkyl;
each of R18 and R19 are independently selected from the group consisting of hydrogen, cyano and aryl;
t is 0 or 1;
each Q is independently selected from the group consisting of CR11 and N;
Y is selected from the group consisting of CH, N and NO;
Z is selected from the group consisting of C-benzyl, NH, N-benzyl, N-alkyl, O and S;
each V is independently selected from the group consisting of N and CR17; and; R17 is selected from the group consisting of hydrogen, alkyl, cycloalkyl, aryl, and thioalkyl.

In another preferred embodiment of the present invention, R5 is selected from the group consisting of:

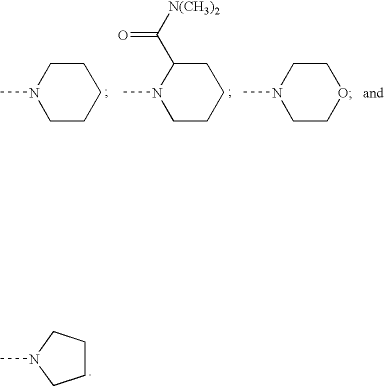

Specific preferred embodiments of the invention include the following compounds: 1′-[4-(benzoyl-methyl-amino)-3-(3,4-dichlorophenyl)-butyl]-[1,4′]bipiperidinyl-4′-carboxylic acid dimethylamide; 1′-[4-(benzoyl-methyl-amino)-3-(3,4-dichlorophenyl)-butyl]-[1,4′]bipiperidinyl-4′-carboxylic acid methylamide; 1′-[4-(cyclohexanecarbonyl-methyl-amino)-3-(3,4-dichlorophenyl)-butyl]-[1,4′]bipiperidinyl-4′-carboxylic acid dimethylamide; 1′-{3-(3,4-dichlorophenyl)-4-[(4-fluoro-benzoyl)-methyl-amino]-butyl}-[1,4′]bipiperidinyl-4′-carboxylic acid dimethylamide; acetic acid 4-{[2-(3,4-dichlorophenyl)-4-(4′-dimethylcarbamoyl-[1,4′]bi-piperidinyl-1′-yl)-butyl]-methyl-carbamoyl}-phenyl ester; 1′-{3-(3,4-dichlorophenyl)-4-[(4-hydroxy-benzoyl)-methyl-amino]-butyl}-[1,4′]bi-piperidinyl-4′-carboxylic acid dimethylamide; acetic acid 2-{[2-(3,4-dichlorophenyl)-4-(4′-dimethylcarbamoyl-[1,4′]bi-piperidinyl-1′-yl)-butyl]-methyl-carbamoyl}-phenyl ester; 1′-[4-[(3-chloro-4-fluoro-benzoyl)-methyl-amino]-3-(3,4-dichlorophenyl)-butyl]-[1,4′]bipiperidinyl-4′-carboxylic acid dimethylamide; 1′-{3-(3,4-dichlorophenyl)-4-[(3,5-difluoro-benzoyl)-methyl-amino]-butyl}-[1,4′]bipiperidinyl-4′-carboxylic acid dimethylamide; 1′-[4-[(5-chloro-2-fluoro-benzoyl)-methyl-amino]-3-(3,4-dichlorophenyl)-butyl]-[1,4′]bipiperidinyl-4′-carboxylic acid dimethylamide; 1′-{3-(3,4-dichlorophenyl)-4-[methyl-(naphthalene-1-carbonyl-3-cyano)-amino]-butyl}-[1,4′]bipiperidinyl-4′-carboxylic acid dimethylamide; 1′-{3-(3,4-dichlorophenyl)-4-[(2-hydroxy-benzoyl)-methyl-amino]-butyl}-[1,4′]bipiperidinyl-4′-carboxylic acid dimethylamide; 1′-{3-(3,4-dichlorophenyl)-4-[(2,4-difluoro-benzoyl)-methyl-amino]-butyl}-[1,4′]bipiperidinyl-4′-carboxylic acid dimethylamide; 1′-{3-(3,4-dichlorophenyl)-4-[(3,4-difluoro-benzoyl)-methyl-amino]-butyl}-[1,4′]bipiperidinyl-4′-carboxylic acid dimethylamide; 1′-{3-(3,4-dichlorophenyl)-4-[(2,5-difluoro-benzoyl)-methyl-amino]-butyl}-[1,4′]bipiperidinyl-4′-carboxylic acid dimethylamide; 1′-{3-(3,4-dichlorophenyl)-4-[methyl-(2,3,4-trifluoro-benzoyl)-amino]-butyl}-[1,4′]bipiperidinyl-4′-carboxylic acid dimethylamide; 1′-{3-(3,4-dichlorophenyl)-4-[methyl-(1-oxy-pyridine-4-carbonyl)-amino]-butyl}-[1,4′]bipiperidinyl-4′-carboxylic acid dimethylamide; 1′-[4-[(6-chloro-pyridine-3-carbonyl)-methyl-amino]-3-(3,4-dichlorophenyl)-butyl]-[1,4′]bipiperidinyl-4′-carboxylic acid dimethylamide; 1′-{3-(3,4-dichlorophenyl)-4-[methyl-(pyridine-3-carbonyl)-amino]-butyl}-[1,4′]bipiperidinyl-4′-carboxylic acid dimethylamide; 1′-[4-[(3-benzyl-2-methylsulfanyl-3H-imidazole-4-carbonyl)-methyl-amino]-3-(3,4-dichlorophenyl)-butyl]-[1,4′]bipiperidinyl-4′-carboxylic acid dimethylamide; 1′-{3-(3,4-dichlorophenyl)-4-[methyl-(4-oxo-2-phenyl-4H-chromene-3-carbonyl)-amino]-butyl}-[1,4′]bipiperidinyl-4′-carboxylic acid dimethylamide; 1′-[4-(cyclopropanecarbonyl-methyl-amino)-3-(3,4-dichlorophenyl)-butyl]-[1,4′]bi-piperidinyl-4′-carboxylic acid dimethylamide; 1′-[4-(cyclopentanecarbonyl-methyl-amino)-3-(3,4-dichlorophenyl)-butyl]-[1,4′]bipiperidinyl-4′-carboxylic acid dimethylamide; 1′-{3-(3,4-dichlorophenyl)-4-[methyl-(2-methylamino-benzoyl)-amino]-butyl}-[1,4′]bipiperidinyl-4′-carboxylic acid dimethylamide; N-[2-(3,4-dichlorophenyl)-4-(4′-dimethylcarbamoyl-[1,4′]bipiperidinyl-1′-yl)-butyl]-N-methyl-phthalamic acid; 1′-{3-(3,4-dichlorophenyl)-4-[(4-methoxy-benzoyl)-methyl-amino]-butyl}-[1,4′]bipiperidinyl-4′-carboxylic acid dimethylamide; 1′-[4-[(biphenyl-4-carbonyl)-methyl-amino]-3-(3,4-dichlorophenyl)-butyl]-[1,4′]bipiperidinyl-4′-carboxylic acid dimethylamide; 1′-{3-(3,4-dichlorophenyl)-4-[(3,3-diphenyl-propionyl)-methyl-amino]-butyl}-[1,4′]bipiperidinyl-4′-carboxylic acid dimethylamide; 1′-(3-(3,4-dichlorophenyl)-4-{[3-(4-hydroxy-phenyl)-propionyl]-methyl-amino}-butyl)-[1,4′]bipiperidinyl-4′-carboxylic acid dimethylamide; 1′-{3-(3,4-dichlorophenyl)-4-[methyl-(1-methyl-1H-pyrrole-2-carbonyl)-amino]-butyl}-[1,4′]bipiperidinyl-4′-carboxylic acid dimethylamide; 1′-{3-(3,4-dichlorophenyl)-4-[(furan-2-carbonyl)-methyl-amino]-butyl}-[1,4′]bipiperidinyl-4′-carboxylic acid dimethylamide; 1′-{3-(3,4-dichlorophenyl)-4-[methyl-(naphthalene-2-carbonyl)-amino]-butyl}-[1,4′]bipiperidinyl-4′-carboxylic acid dimethylamide; 1′-[4-[(2-biphenyl-4-yl-acetyl)-methyl-amino]-3-(3,4-dichlorophenyl)-butyl]-[1,4′]bipiperidinyl-4′-carboxylic acid dimethylamide; 1′-[4-{[3-(4-chlorophenyl)-acryloyl]-methyl-amino}-3-(3,4-dichlorophenyl)-butyl]-[1,4′]bipiperidinyl-4′-carboxylic acid dimethylamide; 1′-{3-(3,4-dichlorophenyl)-4-[methyl-(1H-pyrrole-2-carbonyl)-amino]-butyl}-[1,4′]bipiperidinyl-4′-carboxylic acid dimethylamide; 1′-{3-(3,4-dichlorophenyl)-4-[(furan-2-carbonyl)-methyl-amino]-butyl}-[1,4′]bipiperidinyl-4′-carboxylic acid dimethylamide; 1′-{3-(3,4-dichlorophenyl)-4-[methyl-(thiophene-2-carbonyl)-amino]-butyl}-[1,4′]bipiperidinyl-4′-carboxylic acid dimethylamide; 1′-{3-(3,4-dichlorophenyl)-4-[methyl-(thiophene-3-carbonyl)-amino]-butyl}-[1,4′]bipiperidinyl-4′-carboxylic acid dimethylamide; 1′-{3-(3,4-dichlorophenyl)-4-[(1H-indole-3-carbonyl)-methyl-amino]-butyl}-[1,4′]bipiperidinyl-4′-carboxylic acid dimethylamide; 1′-{3-(3,4-dichlorophenyl)-4-[(2-1H-indol-3-yl-acetyl)-methyl-amino]-butyl}-[1,4′]bipiperidinyl-4′-carboxylic acid dimethylamide; 1′-{3-(3,4-dichlorophenyl)-4-[(1H-indole-5-carbonyl)-methyl-amino]-butyl}-[1,4′]bipiperidinyl-4′-carboxylic acid dimethylamide; 1′-{3-(3,4-dichlorophenyl)-4-[methyl-(pyrazine-2-carbonyl)-amino]-butyl}-[1,4′]bipiperidinyl-4′-carboxylic acid dimethylamide; 1′-{3-(3,4-dichlorophenyl)-4-[methyl-(pyridine-2-carbonyl)-amino]-butyl}-[1,4′]bipiperidinyl-4′-carboxylic acid dimethylamide; 1′-{3-(3,4-dichlorophenyl)-4-[methyl-(pyridine-4-carbonyl)-amino]-butyl}-[1,4′]bipiperidinyl-4′-carboxylic acid dimethylamide; 1′-{3-(3,4-dichlorophenyl)-4-[methyl-(4-oxo-4H-chromene-2-carbonyl)-amino]-butyl}-[1,4′]bipiperidinyl-4′-carboxylic acid dimethylamide; 1′-{3-(3,4-dichlorophenyl)-4-[methyl-(4-sulfamoyl-benzoyl)-amino]-butyl}-[1,4′]bipiperidinyl-4′-carboxylic acid dimethylamide; 1′-[4-[(4-chloro-3-sulfamoyl-benzoyl)-methyl-amino]-3-(3,4-dichlorophenyl)-butyl]-[1,4′]bipiperidinyl-4′-carboxylic acid dimethylamide; 1′-{3-(3,4-dichlorophenyl)-4-[(2-1H-imidazol-4-yl-acetyl)-methyl-amino]-butyl}-[1,4′]bipiperidinyl-4′-carboxylic acid dimethylamide; 1′-{3-(3,4-dichlorophenyl)-4-[methyl-(2-pyridin-2-yl-acetyl)-amino]-butyl}-[1,4′]bipiperidinyl-4′-carboxylic acid dimethylamide; 1′-{3-(3,4-dichlorophenyl)-4-[methyl-(2-pyridin-3-yl-acetyl)-amino]-butyl}-[1,4′]bipiperidinyl-4′-carboxylic acid dimethylamide; 1′-{3-(3,4-dichlorophenyl)-4-[methyl-(2-pyridin-4-yl-acetyl)-amino]-butyl}-[1,4′]bipiperidinyl-4′-carboxylic acid dimethylamide; 1′-[4-[(1-acetylpiperidine-4-carbonyl)-methylamino]-3-(3,4-dichlorophenyl)-butyl]-[1,4′]bipiperidinyl-4′-carboxylic acid dimethylamide; 1′-{3-(3,4-dichlorophenyl)-4-[methyl-(tetrahydropyran-4-carbonyl)-amino]-butyl}-[1,4′]bi-piperidinyl-4′-carboxylic acid dimethylamide; (4-{[2-(3,4-dichlorophenyl)-4-(4′-dimethylcarbamoyl-[1,4′]bipiperidinyl-1′-yl)-butyl]-methyl-carbamoyl}-phenyl)-carbamic acid tert-butyl ester; 1′-{3-(3,4-dichlorophenyl)-4-[(3-{trifluoromethyl-methoxy}-benzoyl)-methyl-amino]-butyl}-[1,4′]bipiperidinyl-4′-carboxylic acid dimethylamide; 1′-(3-(3,4-dichlorophenyl)-4-{[2-(2,4-di{trifluoromethyl}-phenyl)-acetyl]-methylamino}-butyl)-[1,4′]bipiperidinyl-4′-carboxylic acid dimethylamide; 1′-(3-(3,4-dichlorophenyl)-4-{[2-(2,6-dihydroxypyrimidin-4-yl)-acetyl]-methyl-amino}-butyl)-[1,4′]bipiperidinyl-4′-carboxylic acid dimethylamide; 4-{[2-(3,4-dichlorophenyl)-4-(4′-dimethylcarbamoyl-[1,4′]bipiperidinyl-1′-yl)-butyl]-methyl-carbamoyl}-piperidine-1-carboxylic acid tert-butyl ester; 1′-{3-(3,4-dichlorophenyl)-4-[(1H-imidazole-4-carbonyl)-methyl-amino]-butyl}-[1,4′]bipiperidinyl-4′-carboxylic acid dimethylamide; (1-{[2-(3,4-dichlorophenyl)-4-(4′-dimethylcarbamoyl-[1,4′]bipiperidinyl-1′-yl)-butyl]-methyl-carbamoyl}-2-phenyl-ethyl)-carbamic acid tert-butyl ester; [2-(3,4-dichlorophenyl)-4-(4′-dimethylcarbamoyl-[1,4′]bipiperidinyl-1′-yl)-butyl]-methyl-carbamic acid tert-butyl ester; 1′-{3-(3,4-dichlorophenyl)-4-[(furazan-3-carbonyl)-methyl-amino]-butyl}-[1,4′]bipiperidinyl-4′-carboxylic acid dimethylamide; 1′-{3-(3,4-dichlorophenyl)-4-[(2,2-difluorobenzo[1,3]dioxole-5-carbonyl)-methyl-amino]-butyl}-[1,4′]bipiperidinyl-4′-carboxylic acid dimethylamide; 1′-{3-(3,4-dichlorophenyl)-4-[methyl-(1H-pyrrole-3-carbonyl)-amino]-butyl}-[1,4′]bipiperidinyl-4′-carboxylic acid dimethylamide; 1′-(3-(3,4-dichlorophenyl)-4-{[3-(4-fluorophenyl)-5-methyl-isoxazole-4-carbonyl]-methyl-amino}-butyl)-[1,4′]bipiperidinyl-4′-carboxylic acid dimethylamide; 1′-(3-(3,4-dichlorophenyl)-4-{[5-(4-methoxyphenyl)-oxazole-4-carbonyl]-methylamino}-butyl)-[1,4′]bipiperidinyl-4′-carboxylic acid dimethylamide; 1′-{3-(3,4-dichlorophenyl)-4-[methyl-(5-methyl-1-phenyl-1H-[1,2,3]triazole-4-carbonyl)-amino]-butyl}-[1,4′]bipiperidinyl-4′-carboxylic acid dimethylamide; 1′-[4-[(benzofuran-5-carbonyl)-methyl-amino]-3-(3,4-dichlorophenyl)-butyl]-[1,4′]bipiperidinyl-4′-carboxylic acid dimethylamide; 1′-{3-(3,4-dichlorophenyl)-4-[methyl-(5-methylbenzo[b]thiophene-2-carbonyl)-amino]-butyl}-[1,4′]bipiperidinyl-4′-carboxylic acid dimethylamide; 1′-[4-[(3,5-bis-trifluoromethyl-benzoyl)-methylamino]-3-(3,4-dichlorophenyl)-butyl]-[1,4′]bipiperidinyl-4′-carboxylic acid dimethylamide; 1′-[4-[(2-bromobenzoyl)-methylamino]-3-(3,4-dichlorophenyl)-butyl]-[1,4′]bipiperidinyl-4′-carboxylic acid dimethylamide; 1′-{3-(3,4-dichlorophenyl)-4-[(2-fluoro-benzoyl)-methylamino]-butyl}-[1,4′]bipiperidinyl-4′-carboxylic acid dimethylamide; 1′-[3-(3,4-dichlorophenyl)-4-(methyl-pentafluorobenzoyl-amino)-butyl]-[1,4′]bipiperidinyl-4′-carboxylic acid dimethylamide; 1′-{3-(3,4-dichlorophenyl)-4-[(2,6-difluorobenzoyl)-methylamino]-butyl}-[1,4′]bipiperidinyl-4′-carboxylic acid dimethylamide; 1′-[4-[(2,4-dichlorobenzoyl)-methyl-amino]-3-(3,4-dichlorophenyl)-butyl]-[1,4′]bipiperidinyl-4′-carboxylic acid dimethylamide; 1′-[4-[(2,6-dichlorobenzoyl)-methylamino]-3-(3,4-dichlorophenyl)-butyl]-[1,4′]bipiperidinyl-4′-carboxylic acid dimethylamide; 1′-{3-(3,4-dichlorophenyl)-4-[methyl-(2-trifluoromethyl-benzoyl)-amino]-butyl}-[1,4′]bipiperidinyl-4′-carboxylic acid dimethylamide; 1′-{3-(3,4-dichlorophenyl)-4-[methyl-(2-methylbenzoyl)-amino]-butyl}-[1,4′]bipiperidinyl-4′-carboxylic acid dimethylamide; 1′-{3-(3,4-dichlorophenyl)-4-[(3-fluorobenzoyl)-methylamino]-butyl}-[1,4′]bipiperidinyl-4′-carboxylic acid dimethylamide; 1′-[4-[(3-chlorobenzoyl)-methylamino]-3-(3,4-dichlorophenyl)-butyl]-[1,4′]bi-piperidinyl-4′-carboxylic acid dimethylamide; 1′-[4-[(3,4-dichlorobenzoyl)-methylamino]-3-(3,4-dichlorophenyl)-butyl]-[1,4′]bipiperidinyl-4′-carboxylic acid dimethylamide; 1′-{3-(3,4-dichlorophenyl)-4-[(3-methoxybenzoyl)-methylamino]-butyl}-[1,4′]bipiperidinyl-4′-carboxylic acid dimethylamide; 1′-{3-(3,4-dichlorophenyl)-4-[methyl-(3-trifluoromethyl-benzoyl)-amino]-butyl}-[1,4′]bipiperidinyl-4′-carboxylic acid dimethylamide; 1′-[4-[(4-chlorobenzoyl)-methylamino]-3-(3,4-dichlorophenyl)-butyl]-[1,4′]bipiperidinyl-4′-carboxylic acid dimethylamide; 1′-{3-(3,4-dichlorophenyl)-4-[(4-methoxybenzoyl)-methyl-amino]-butyl}-[1,4′]bipiperidinyl-4′-carboxylic acid dimethylamide; 1′-{3-(3,4-dichlorophenyl)-4-[methyl-(4-trifluoromethyl-benzoyl)-amino]-butyl}-[1,4′]bipiperidinyl-4′-carboxylic acid dimethylamide; 1′-{3-(3,4-dichlorophenyl)-4-[(4-methylbenzoyl)-methylamino]-butyl}-[1,4′]bipiperidinyl-4′-carboxylic acid dimethylamide; 1′-{3-(3,4-dichlorophenyl)-4-[(2,2-dimethylpropionyl)-methylamino]-butyl}-[1,4′]bipiperidinyl-4′-carboxylic acid dimethylamide; 1′-[3-(3,4-dichlorophenyl)-4-(methyl-phenylacetyl-amino)-butyl]-[1,4′]bipiperidinyl-4′-carboxylic acid dimethylamide; 1′-{3-(3,4-dichlorophenyl)-4-[methyl-(2-phenyl-cyclopropanecarbonyl)-amino]-butyl}-[1,4′]bipiperidinyl-4′-carboxylic acid dimethylamide; 1′-[4-[(4-cyanobenzoyl)-methylamino]-3-(3,4-dichlorophenyl)-butyl]-[1,4′]bipiperidinyl-4′-carboxylic acid dimethylamide; 1′-{3-(3,4-dichlorophenyl)-4-[methyl-(naphthalene-1-carbonyl)-amino]-butyl}-[1,4′]bipiperidinyl-4′-carboxylic acid dimethylamide; 1′-[4-[(3-cyano-naphthalene-1-carbonyl)-methylamino]-3-(3,4-dichlorophenyl)-butyl]-[1,4′]bipiperidinyl-4′-carboxylic acid dimethylamide; 1′-[4-(benzoyl-methylamino)-3-phenyl-butyl]-[1,4′]bipiperidinyl-4′-carboxylic acid dimethylamide; 1′-[4-(benzoyl-methylamino)-3-(3,4-dichlorophenyl)-butyl]-[1,4′]bipiperidinyl-4′-carboxylic acid ethyl-methyl-amide; N-{2-(3,4-dichlorophenyl)-4-[4-(1-dimethylcarbamoyl-cyclohexyl)-piperazin-1-yl]-butyl}-N-methyl-benzamide; 1′-[4-(benzoyl-methylamino)-3-(3,4-dichlorophenyl)-butyl]-[1,4′]bipiperidinyl-2-carboxylic acid dimethylamide; 1-[4-[(3-cyano-naphthalene-1-carbonyl)-methylamino]-3-(3,4-dichlorophenyl)-butyl]-4-pyrrolidin-1-yl-piperidine-4-carboxylic acid dimethylamide; 1-[4-(benzoyl-methylamino)-3-(3,4-dichlorophenyl)-butyl]-4-pyrrolidin-1-yl-piperidine-4-carboxylic acid dimethylamide; N-[4-[4-(cyclopropylmethyl-propionyl-amino)-piperidin-1-yl]-2-(3,4-dichlorophenyl)-butyl]-N-methyl-benzamide; N-{2-(3,4-dichlorophenyl)-4-[4-(isopropyl-propionyl-amino)-piperidin-1-yl]-butyl}-N-methyl-benzamide; N-{2-(3,4-dichlorophenyl)-4-[4-(phenyl-propionyl-amino)-piperidin-1-yl]-butyl}-N-methyl-benzamide; N-[4-[4-(butyl-propionyl-amino)-piperidin-1-yl]-2-(3,4-dichlorophenyl)-butyl]-N-methyl-benzamide; N-[4-[4-(butyl-cyclopropanecarbonyl-amino)-piperidin-1-yl]-2-(3,4-dichlorophenyl)-butyl]-N-methyl-benzamide; N-[4-[4-(butyl-cyclohexanecarbonyl-amino)-piperidin-1-yl]-2-(3,4-dichlorophenyl)-butyl]-N-methyl-benzamide; N-[4-[4-(benzoyl-butyl-amino)-piperidin-1-yl]-2-(3,4-dichlorophenyl)-butyl]-N-methyl-benzamide; N-(2-(3,4-dichlorophenyl)-4-{4-[(4-methoxybutyl)-propionyl-amino]-piperidin-1-yl}-butyl)-N-methyl-benzamide; N-[4-{4-[cyclopropanecarbonyl-(4-methoxybutyl)-amino]-piperidin-1-yl}-2-(3,4-dichlorophenyl)-butyl]-N-methyl-benzamide; N-[4-{4-[cyclohexanecarbonyl-(4-methoxybutyl)-amino]-piperidin-1-yl}-2-(3,4-dichlorophenyl)-butyl]-N-methyl-benzamide; N-[4-{4-[benzoyl-(4-methoxybutyl)-amino]-piperidin-1-yl}-2-(3,4-dichlorophenyl)-butyl]-N-methyl-benzamide; N-[4-{4-[cyclohexyl(propionyl)amino]piperidin-1-yl}-2-(3,4-dichlorophenyl)butyl]-N-methylbenzamide; N-[4-{4-[cyclohexyl(cyclopropylcarbonyl)-amino]piperidin-1-yl}-2-(3,4-dichlorophenyl)butyl]-N-methylbenzamide; N-[4-{4-[cyclo-hexyl(cyclohexylcarbonyl)amino]piperidin-1-yl}-2-(3,4-dichlorophenyl)butyl]-N-methyl-benzamide; N-[4-{4-[benzoyl(cyclohexyl)amino]piperidin-1-yl}-2-(3,4-dichlorophenyl)-butyl]-N-methylbenzamide; N-[2-(3,4-dichlorophenyl)-4-{4-[(1-methylpiperidin-4-yl)-(propionyl)amino]piperidin-1-yl}butyl]-N-methylbenzamide; N-[4-{4-[(cyclopropyl-carbonyl)(1-methylpiperidin-4-yl)amino]piperidin-1-yl}-2-(3,4-dichlorophenyl)butyl]-N-methylbenzamide; N-[4-{4-[(cyclohexylcarbonyl)(1-methylpiperidin-4-yl)amino]piperidin-1-yl}-2-(3,4-dichlorophenyl)butyl]-N-methylbenzamide; N-{1-[4-[benzoyl(methyl)amino]-3-(3,4-dichlorophenyl)butyl]piperidin-4-yl}-N-(1-methylpiperidin-4-yl)benzamide; N-{2-(3,4-dichlorophenyl)-4-[4′-(pyrrolidine-1-carbonyl)-[1,4′]bipiperidinyl-1′-yl]-butyl}-N-methyl-benzamide; 1-[4-[(3-cyano-naphthalene-1-carbonyl)-methylamino]-3-(3,4-dichlorophenyl)-butyl]-4-(2-oxo-pyrrolidin-1-yl)-piperidine-4-carboxylic acid dimethylamide; 3-cyano-naphthalene-1-carboxylic acid {2-(3,4-dichlorophenyl)-4-[4′-(piperidine-1-carbonyl)-[1,4′]bipiperidinyl-1′-yl]-butyl}-methyl-amide; 1′-[4-(benzoyl-methyl-amino)-3-(3,4-dichlorophenyl)-butyl]-[1,4′]bipiperidinyl-4′-carboxylic acid dipropylamide; 1-[4-(benzoyl-methylamino)-3-(3,4-dichlorophenyl)-butyl]-4-morpholin-4-yl-piperidine-4-carboxylic acid dimethylamide; 1-[4-(benzoyl-methyl-amino)-3-(3,4-dichlorophenyl)-butyl]-4-pyrrolidin-1-yl-piperidine-4-carboxylic acid isopropyl-methyl-amide; N-{2-(3,4-dichlorophenyl)-4-[4-(piperidine-1-carbonyl)-4-pyrrolidin-1-yl-piperidin-1-yl]-butyl}-N-methyl-benzamide; 1-[4-(benzoyl-methylamino)-3-(3,4-dichlorophenyl)-butyl]-4-pyrrolidin-1-yl-piperidine-4-carboxylic acid diethylamide; N-{2-(3,4-dichlorophenyl)-4-[4-(morpholine-4-carbonyl)-4-pyrrolidin-1-yl-piperidin-1-yl]-butyl}-N-methyl-benzamide; and physiologically compatible salts, especially acid addition salts, of these compounds.

Particularly preferred are the following compounds: 1′-[4-(benzoyl-methyl-amino)-3-(3,4-dichlorophenyl)-butyl]-[1,4′]bipiperidinyl-4′-carboxylic acid dimethylamide; 1′-[4-(benzoyl-methyl-amino)-3-(3,4-dichlorophenyl)-butyl]-[1,4′]bipiperidinyl-4′-carboxylic acid methylamide; 1′-[4-(cyclohexanecarbonyl-methyl-amino)-3-(3,4-dichlorophenyl)-butyl]-[1,4′]bipiperidinyl-4′-carboxylic acid dimethylamide; 1′-{3-(3,4-dichlorophenyl)-4-[(4-fluoro-benzoyl)-methyl-amino]-butyl}-[1,4′]bipiperidinyl-4′-carboxylic acid dimethylamide; acetic acid 4-{[2-(3,4-dichlorophenyl)-4-(4′-dimethylcarbamoyl-[1,4′]bi-piperidinyl-1′-yl)-butyl]-methyl-carbamoyl}-phenyl ester; 1′-{3-(3,4-dichlorophenyl)-4-[(4-hydroxybenzoyl)-methyl-amino]-butyl}-[1,4′]bi-piperidinyl-4′-carboxylic acid dimethylamide; acetic acid 2-{[2-(3,4-dichlorophenyl)-4-(4′-dimethylcarbamoyl-[1,4′]bi-piperidinyl-1′-yl)-butyl]-methyl-carbamoyl}-phenyl ester; 1′-[4-[(3-chloro-4-fluorobenzoyl)-methyl-amino]-3-(3,4-dichlorophenyl)-butyl]-[1,4′]bipiperidinyl-4′-carboxylic acid dimethylamide; 1′-{3-(3,4-dichlorophenyl)-4-[(3,5-difluorobenzoyl)-methylamino]-butyl}-[1,4′]bipiperidinyl-4′-carboxylic acid dimethylamide; 1′-[4-[(5-chloro-2-fluorobenzoyl)-methylamino]-3-(3,4-dichlorophenyl)-butyl]-[1,4′]bipiperidinyl-4′-carboxylic acid dimethylamide; 1′-{3-(3,4-dichlorophenyl)-4-[methyl-(naphthalene-1-carbonyl-3-cyano)-amino]-butyl}-[1,4′]bipiperidinyl-4′-carboxylic acid dimethylamide; 1′-{3-(3,4-dichlorophenyl)-4-[(2-hydroxybenzoyl)-methylamino]-butyl}-[1,4′]bipiperidinyl-4′-carboxylic acid dimethylamide; 1′-{3-(3,4-dichlorophenyl)-4-[(2,4-difluorobenzoyl)-methyl-amino]-butyl}-[1,4′]bipiperidinyl-4′-carboxylic acid dimethylamide; 1′-{3-(3,4-dichlorophenyl)-4-[(3,4-difluorobenzoyl)-methylamino]-butyl}-[1,4′]bipiperidinyl-4′-carboxylic acid dimethylamide; 1′-{3-(3,4-dichlorophenyl)-4-[(2,5-difluorobenzoyl)-methylamino]-butyl}-[1,4′]bipiperidinyl-4′-carboxylic acid dimethylamide; 1′-{3-(3,4-dichlorophenyl)-4-[methyl-(2,3,4-trifluoro-benzoyl)-amino]-butyl}-[1,4′]bipiperidinyl-4′-carboxylic acid dimethylamide; 1′-[4-[(6-chloropyridine-3-carbonyl)-methylamino]-3-(3,4-dichlorophenyl)-butyl]-[1,4′]bipiperidinyl-4′-carboxylic acid dimethylamide; 1′-{3-(3,4-dichlorophenyl)-4-[methyl-(pyridine-3-carbonyl)-amino]-butyl}-[1,4′]bipiperidinyl-4′-carboxylic acid dimethylamide; 1′-[4-(cyclopentanecarbonyl-methylamino)-3-(3,4-dichlorophenyl)-butyl]-[1,4′]bipiperidinyl-4′-carboxylic acid dimethylamide; 1′-{3-(3,4-dichlorophenyl)-4-[methyl-(2-methylamino-benzoyl)-amino]-butyl}-[1,4′]bipiperidinyl-4′-carboxylic acid dimethylamide; N-[2-(3,4-dichlorophenyl)-4-(4′-dimethylcarbamoyl-[1,4′]bipiperidinyl-1′-yl)-butyl]-N-methyl-phthalamic acid; 1′-{3-(3,4-dichlorophenyl)-4-[(4-methoxybenzoyl)-methyl-amino]-butyl}-[1,4′]bipiperidinyl-4′-carboxylic acid dimethylamide; 1′-(3-(3,4-dichlorophenyl)-4-{[3-(4-hydroxyphenyl)-propionyl]-methylamino}-butyl)-[1,4′]bipiperidinyl-4′-carboxylic acid dimethylamide; 1′-{3-(3,4-dichlorophenyl)-4-[methyl-(1-methyl-1H-pyrrole-2-carbonyl)-amino]-butyl}-[1,4′]bipiperidinyl-4′-carboxylic acid dimethylamide; 1′-{3-(3,4-dichlorophenyl)-4-[(furan-2-carbonyl)-methylamino]-butyl}-[1,4′]bipiperidinyl-4′-carboxylic acid dimethylamide; 1′-{3-(3,4-dichlorophenyl)-4-[methyl-(naphthalene-2-carbonyl)-amino]-butyl}-[1,4′]bipiperidinyl-4′-carboxylic acid dimethylamide; 1′-{3-(3,4-dichlorophenyl)-4-[methyl-(1H-pyrrole-2-carbonyl)-amino]-butyl}-[1,4′]bipiperidinyl-4′-carboxylic acid dimethylamide; 1′-{3-(3,4-dichlorophenyl)-4-[(furan-2-carbonyl)-methyl-amino]-butyl}-[1,4′]bipiperidinyl-4′-carboxylic acid dimethylamide; 1′-{3-(3,4-dichlorophenyl)-4-[methyl-(thiophene-2-carbonyl)-amino]-butyl}-[1,4′]bipiperidinyl-4′-carboxylic acid dimethylamide; 1′-{3-(3,4-dichlorophenyl)-4-[methyl-(thiophene-3-carbonyl)-amino]-butyl}-[1,4′]bipiperidinyl-4′-carboxylic acid dimethylamide; 1′-{3-(3,4-dichlorophenyl)-4-[(1H-indole-5-carbonyl)-methyl-amino]-butyl}-[1,4′]bipiperidinyl-4′-carboxylic acid dimethylamide; 1′-{3-(3,4-dichlorophenyl)-4-[methyl-(pyridine-2-carbonyl)-amino]-butyl}-[1,4′]bipiperidinyl-4′-carboxylic acid dimethylamide; 1′-{3-(3,4-dichlorophenyl)-4-[methyl-(pyridine-4-carbonyl)-amino]-butyl}-[1,4′]bipiperidinyl-4′-carboxylic acid dimethylamide; 1′-{3-(3,4-dichlorophenyl)-4-[methyl-(4-oxo-4H-chromene-2-carbonyl)-amino]-butyl}-[1,4′]bipiperidinyl-4′-carboxylic acid dimethylamide; 1′-{3-(3,4-dichlorophenyl)-4-[methyl-(4-sulfamoyl-benzoyl)-amino]-butyl}-[1,4′]bipiperidinyl-4′-carboxylic acid dimethylamide; 1′-[4-[(4-chloro-3-sulfamoyl-benzoyl)-methylamino]-3-(3,4-dichlorophenyl)-butyl]-[1,4′]bipiperidinyl-4′-carboxylic acid dimethylamide; 1′-{3-(3,4-dichlorophenyl)-4-[methyl-(2-pyridin-2-yl-acetyl)-amino]-butyl}-[1,4′]bipiperidinyl-4′-carboxylic acid dimethylamide; 1′-{3-(3,4-dichlorophenyl)-4-[methyl-(2-pyridin-3-yl-acetyl)-amino]-butyl}-[1,4′]bipiperidinyl-4′-carboxylic acid dimethylamide; 1′-{3-(3,4-dichlorophenyl)-4-[methyl-(2-pyridin-4-yl-acetyl)-amino]-butyl}-[1,4′]bipiperidinyl-4′-carboxylic acid dimethylamide; 1′-{3-(3,4-dichlorophenyl)-4-[methyl-(tetrahydropyran-4-carbonyl)-amino]-butyl}-[1,4′]bipiperidinyl-4′-carboxylic acid dimethylamide; (4-{[2-(3,4-dichlorophenyl)-4-(4′-dimethylcarbamoyl-[1,4′]bipiperidinyl-1′-yl)-butyl]-methyl-carbamoyl}-phenyl)-carbamic acid tert-butyl ester; 1′-{3-(3,4-dichlorophenyl)-4-[(3-{trifluoromethyl-methoxy}-benzoyl)-methyl-amino]-butyl}-[1,4′]bipiperidinyl-4′-carboxylic acid dimethylamide; 1′-(3-(3,4-dichlorophenyl)-4-{[2-(2,4-di{trifluoromethyl}-phenyl)-acetyl]-methyl-amino}-butyl)-[1,4′]bipiperidinyl-4′-carboxylic acid dimethylamide; 1′-{3-(3,4-dichlorophenyl)-4-[(2,2-difluorobenzo[1,3]dioxole-5-carbonyl)-methyl-amino]-butyl}-[1,4′]bipiperidinyl-4′-carboxylic acid dimethylamide; 1′-{3-(3,4-dichlorophenyl)-4-[methyl-(5-methyl-1-phenyl-1H-[1,2,3]triazole-4-carbonyl)-amino]-butyl}-[1,4′]bipiperidinyl-4′-carboxylic acid dimethylamide; 1′-[4-[(benzofuran-5-carbonyl)-methyl-amino]-3-(3,4-dichlorophenyl)-butyl]-[1,4′]bipiperidinyl-4′-carboxylic acid dimethylamide; 1′-[4-[(2-bromobenzoyl)-methylamino]-3-(3,4-dichlorophenyl)-butyl]-[1,4′]bipiperidinyl-4′-carboxylic acid dimethylamide; 1′-{3-(3,4-dichlorophenyl)-4-[(2-fluorobenzoyl)-methylamino]-butyl}-[1,4′]bipiperidinyl-4′-carboxylic acid dimethylamide; 1′-{3-(3,4-dichlorophenyl)-4-[(2,6-difluorobenzoyl)-methylamino]-butyl}-[1,4′]bipiperidinyl-4′-carboxylic acid dimethylamide; 1′-[4-[(2,4-dichloro-benzoyl)-methyl-amino]-3-(3,4-dichlorophenyl)-butyl]-[1,4′]bipiperidinyl-4′-carboxylic acid dimethylamide; 1′-{3-(3,4-dichlorophenyl)-4-[methyl-(2-trifluoromethylbenzoyl)-amino]-butyl}-[1,4′]bipiperidinyl-4′-carboxylic acid dimethylamide; 1′-{3-(3,4-dichlorophenyl)-4-[methyl-(2-methylbenzoyl)-amino}-butyl]-[1,4′]bipiperidinyl-4′-carboxylic acid dimethylamide; 1′-{3-(3,4-dichlorophenyl)-4-[(3-fluorobenzoyl)-methyl-amino]-butyl}-[1,4′]bipiperidinyl-4′-carboxylic acid dimethylamide; 1′-[4-[(3-chloro-benzoyl)-methyl-amino]-3-(3,4-dichlorophenyl)-butyl]-[1,4′]bipiperidinyl-4′-carboxylic acid dimethylamide; 1′-{3-(3,4-dichlorophenyl)-4-[(3-methoxybenzoyl)-methylamino]-butyl}-[1,4′]bipiperidinyl-4′-carboxylic acid dimethylamide; 1′-{3-(3,4-dichlorophenyl)-4-[methyl-(3-trifluoromethylbenzoyl)-amino]-butyl}-[1,4′]bipiperidinyl-4′-carboxylic acid dimethylamide; 1′-{3-(3,4-dichlorophenyl)-4-[(4-methyl-benzoyl)-methylamino]-butyl}-[1,4′]bipiperidinyl-4′-carboxylic acid dimethylamide; 1′-[4-[(4-cyanobenzoyl)-methylamino]-3-(3,4-dichlorophenyl)-butyl]-[1,4′]bipiperidinyl-4′-carboxylic acid dimethylamide; 1′-[4-[(3-cyano-naphthalene-1-carbonyl)-methyl-amino]-3-(3,4-dichlorophenyl)-butyl]-[1,4′]bipiperidinyl-4′-carboxylic acid dimethylamide; 1′-[4-(benzoyl-methyl-amino)-3-phenyl-butyl]-[1,4′]bipiperidinyl-4′-carboxylic acid dimethylamide; 1′-[4-(benzoyl-methyl-amino)-3-(3,4-dichlorophenyl)-butyl]-[1,4′]bipiperidinyl-4′-carboxylic acid ethyl-methyl-amide; N-{2-(3,4-dichlorophenyl)-4-[4-(1-dimethylcarbamoyl-cyclohexyl)-piperazin-1-yl]-butyl}-N-methyl-benzamide; 1′-[4-(benzoyl-methyl-amino)-3-(3,4-dichlorophenyl)-butyl]-[1,4′]bipiperidinyl-2-carboxylic acid dimethylamide; 1-[4-[(3-cyano-naphthalene-1-carbonyl)-methylamino]-3-(3,4-dichlorophenyl)-butyl]-4-pyrrolidin-1-yl-piperidine-4-carboxylic acid dimethylamide; 1-[4-(benzoyl-methyl-amino)-3-(3,4-dichlorophenyl)-butyl]-4-pyrrolidin-1-yl-piperidine-4-carboxylic acid dimethylamide; and physiologically compatible salts, especially acid addition salts, of these compounds.

Most preferred compounds of the present invention include the following: 1′-[4-(benzoyl-methyl-amino)-3-(3,4-dichlorophenyl)-butyl]-[1,4′]bipiperidinyl-4′-carboxylic acid dimethylamide; 1′-[4-(cyclohexanecarbonyl-methylamino)-3-(3,4-dichlorophenyl)-butyl]-[1,4′]bipiperidinyl-4′-carboxylic acid dimethylamide; 1′-{3-(3,4-dichlorophenyl)-4-[(4-fluoro-benzoyl)-methylamino]-butyl}-[1,4′]bipiperidinyl-4′-carboxylic acid dimethylamide; acetic acid 4-{[2-(3,4-dichlorophenyl)-4-(4′-dimethylcarbamoyl-[1,4′]bi-piperidinyl-1′-yl)-butyl]-methyl-carbamoyl}-phenyl ester; 1′-{3-(3,4-dichlorophenyl)-4-[(4-hydroxybenzoyl)-methyl-amino]-butyl}-[1,4′]bi-piperidinyl-4′-carboxylic acid dimethylamide; acetic acid 2-{[2-(3,4-dichlorophenyl)-4-(4′-dimethylcarbamoyl-[1,4′]bi-piperidinyl-1′-yl)-butyl]-methyl-carbamoyl}-phenyl ester; 1′-[4-[(3-chloro-4-fluorobenzoyl)-methylamino]-3-(3,4-dichlorophenyl)-butyl]-[1,4′]bipiperidinyl-4′-carboxylic acid dimethylamide; 1′-{3-(3,4-dichlorophenyl)-4-[(3,5-difluorobenzoyl)-methylamino]-butyl}-[1,4′]bipiperidinyl-4′-carboxylic acid dimethylamide; 1′-[4-[(5-chloro-2-fluorobenzoyl)-methylamino]-3-(3,4-dichlorophenyl)-butyl]-[1,4′]bipiperidinyl-4′-carboxylic acid dimethylamide; 1′-{3-(3,4-dichlorophenyl)-4-[methyl-(naphthalene-1-carbonyl-3-cyano)-amino]-butyl}-[1,4′]bipiperidinyl-4′-carboxylic acid dimethylamide; 1′-{3-(3,4-dichlorophenyl)-4-[(2-hydroxybenzoyl)-methylamino]-butyl}-[1,4′]bipiperidinyl-4′-carboxylic acid dimethylamide; 1′-{3-(3,4-dichlorophenyl)-4-[(2,4-difluorobenzoyl)-methyl-amino]-butyl}-[1,4′]bipiperidinyl-4′-carboxylic acid dimethylamide; 1′-{3-(3,4-dichlorophenyl)-4-[(3,4-difluorobenzoyl)-methylamino]-butyl}-[1,4′]bipiperidinyl-4′-carboxylic acid dimethylamide; 1′-{3-(3,4-dichlorophenyl)-4-[(2,5-difluorobenzoyl)-methylamino]-butyl}-[1,4′]bipiperidinyl-4′-carboxylic acid dimethylamide; 1′-[4-[(6-chloropyridine-3-carbonyl)-methylamino]-3-(3,4-dichlorophenyl)-butyl]-[1,4′]bipiperidinyl-4′-carboxylic acid dimethylamide; 1′-[4-(cyclopentanecarbonyl-methylamino)-3-(3,4-dichlorophenyl)-butyl]-[1,4′]bipiperidinyl-4′-carboxylic acid dimethylamide; 1′-{3-(3,4-dichlorophenyl)-4-[(furan-2-carbonyl)-methylamino]-butyl}-[1,4′]bipiperidinyl-4′-carboxylic acid dimethylamide; 1′-{3-(3,4-dichlorophenyl)-4-[methyl-(pyridine-2-carbonyl)-amino]-butyl}-[1,4′]bipiperidinyl-4′-carboxylic acid dimethylamide; 1′-{3-(3,4-dichlorophenyl)-4-[methyl-(2-pyridin-2-yl-acetyl)-amino]-butyl}-[1,4′]bipiperidinyl-4′-carboxylic acid dimethylamide; 1′-{3-(3,4-dichlorophenyl)-4-[methyl-(2-pyridin-3-yl-acetyl)-amino]-butyl}-[1,4′]bipiperidinyl-4′-carboxylic acid dimethylamide; 1′-{3-(3,4-dichlorophenyl)-4-[(3-{trifluoromethyl-methoxy}-benzoyl)-methyl-amino]-butyl}-[1,4′]bipiperidinyl-4′-carboxylic acid dimethylamide; 1′-{3-(3,4-dichlorophenyl)-4-[methyl-(5-methyl-1-phenyl-1H-[1,2,3]triazole-4-carbonyl)-amino]-butyl}-[1,4′]bipiperidinyl-4′-carboxylic acid dimethylamide; 1′-[4-[(benzofuran-5-carbonyl)-methyl-amino]-3-(3,4-dichlorophenyl)-butyl]-[1,4′]bipiperidinyl-4′-carboxylic acid dimethylamide; 1′-[4-[(3-cyano-naphthalene-1-carbonyl)-methylamino]-3-(3,4-dichlorophenyl)-butyl]-[1,4′]bipiperidinyl-4′-carboxylic acid dimethylamide; 1-[4-[(3-cyano-naphthalene-1-carbonyl)-methylamino]-3-(3,4-dichlorophenyl)-butyl]-4-pyrrolidin-1-yl-piperidine-4-carboxylic acid dimethylamide; and physiologically compatible acid addition salts of these compounds.

The compounds of Formula I and their acid addition salts may be prepared by reacting a compound of formula II 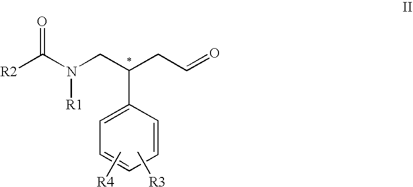
wherein R1 to R4 have the above meanings, with a compound of formula III 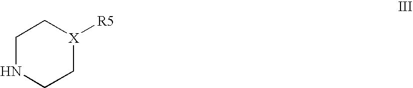
wherein R5 has the above meaning, to result in a compound of formula I which is optionally converted into its physiologically compatible salt.

Alternatively, the compounds of Formula I and their salts may be prepared by reacting a compound of formula III 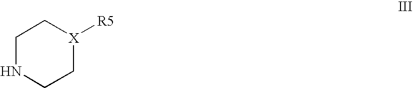
wherein R5 has the above meaning, with a compound of formula IV 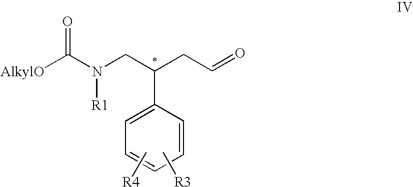
wherein R1, R3 and R4 have the above meanings, to give a compound of formula V,

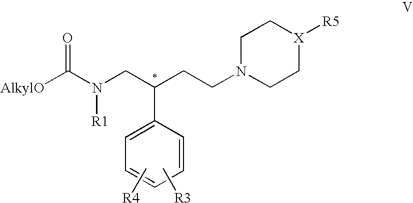

The compound of formula V is then hydrolyzed in an acidic medium to give a compound of formula VI: 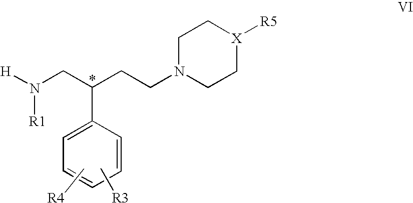
wherein R1 and R3 to R5 have the above meanings. The compound of formula VI is then reacted with a compound of formula VII 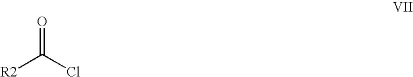
wherein R2 has the above meaning, to result in a compound of formula I which is optionally converted into its physiologically compatible acid addition salt.

Alternatively, the compounds of Formula I and their acid addition salts may be prepared by reacting a compound of formula X 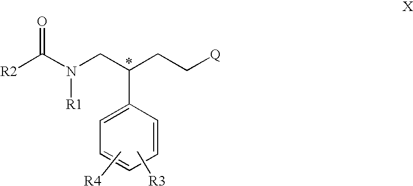
wherein Q is selected from the group consisting of halogen, preferably, bromo or iodo; and methylsulfonyl; with a compound of formula III 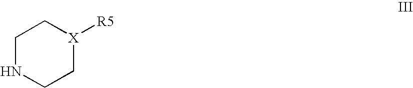
to result in a compound of formula I which is optionally converted into its physiologically compatible acid addition salt.

The compounds of Formula II can be prepared by reacting a compound of formula VII . . . 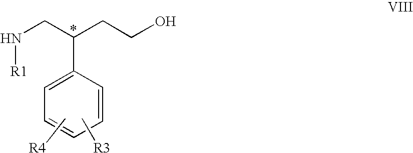
with a compound of formula VII 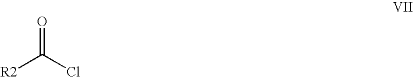
to give a compound of formula IX 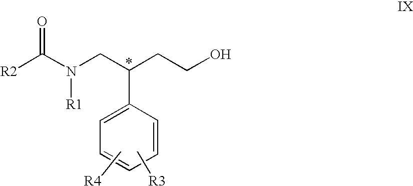
The compound of formula IX is then oxidized to give a compound of formula II.

The compounds of Formula III are well-known and can be prepared as disclosed in any of J. Med. Chem. 1964, p. 619 (van de Westeringh); WO02/094821 (Sanofi); J. Org. Chem. 1980, 45, p. 3671 (J. T. Tai); J. Med. Chem. 1974, 9, p. 424 (B. Devaux); J. Med. Chem. 2002, 45, p. 3972 (J. T. Albert). A reaction scheme for the preparation of compounds of formula II is giving in example 37.

The compounds of Formula IV can be prepared in a similar manner as suggested for compounds of Formula II, by selecting an appropriate R2.

The compounds of Formula VII are known per se or can be prepared by the person skilled in the art from known compounds in known manner.

The compounds of Formula X can be prepared by reacting a compound of formula IX 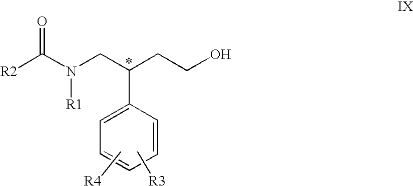
with a hydrogen halide, preferably HBr or HI to deliver compounds of Formula X wherein Q is a halogen, preferably bromo or iodo, or, alternatively, with methanesulfonylchloride to deliver compounds of Formula X wherein Q is methylsulfonyl

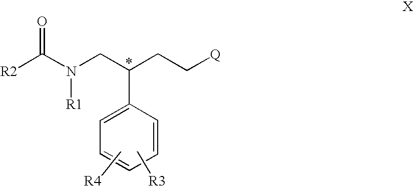

The compounds of Formula I may be isolated from the reaction mixture and purified in known manner. Acid addition salts may be converted into the free bases in conventional manner, and these may if desired be converted in known manner into physiologically compatible acid addition salts. Physiologically compatible salts of compounds of Formula I are their conventional salts with inorganic acids, for example sulfuric acid, phosphoric acids or hydrohalic acids, preferably hydrochloric acid, or with organic acids, for example lower aliphatic monocarboxylic, dicarboxylic or tricarboxylic acids such as maleic acid, fumaric acid, lactic acid, tartaric acid, citric acid, or with sulfonic acids, for example lower alkanesulfonic acids such as methanesulfonic acid or trifluoromethanesulfonic acid, or benzenesulfonic acids optionally substituted in the benzene ring by halogen or lower alkyl, such as p-toluenesulfonic acid.

The compounds of Formula I contain in the γ-position to the ring nitrogen atom in the 4-position of the piperidine or piperazine ring, respectively, an asymmetrical carbon atom, namely the carbon atom *C bearing the phenyl ring substituted by R3 and R4. Hence, the compounds of Formula I may be present in several stereoisomeric forms. The present invention covers both the mixtures of optical isomers and the isomerically pure compounds of Formula I. Preferred are compounds of Formula I in which the carbon atom *C bearing the phenyl ring substituted by R3 and R4 is in the S-configuration. If mixtures of optical isomers of the starting compound, for example of the compounds of Formula II or the compounds of Formula IV, are used in the synthesis of the compounds of Formula I, the compounds of Formula I are also obtained in the form of mixtures of optical isomers. Departing from stereochemically uniform forms of the starting compound, stereochemically uniform compounds of Formula I can also be obtained. The stereochemically uniform compounds of Formula I can be obtained from the mixtures of optical isomers in known manner, for example by chromatographic separation on chiral separating materials or by reaction with suitable optically active acids, for example tartaric acid or 10-camphorsulfonic acid, and subsequent separation into their optically active antipodes by fractional crystallisation of the diastereomeric salts obtained.

The compounds of Formula I and their acid addition salts have properties which are antagonistic to tachykinin receptors and are therefore suitable for the treatment of pathological conditions in larger mammals, particularly humans, in which tachykinins are involved as transfer agents. The group of compounds according to the invention is distinguished by a particularly beneficial action profile which is characterised by a high selectivity to NK2 and/or NK3-receptors. Furthermore, the group of compounds according to the invention is distinguished by good compatibility even over prolonged periods of administration, and by comparatively good oral availability. Due to their activity profile, the compounds of Formula I are suitable in particular for inhibiting processes in which tachykinins, such as neurokinin A, which bind to NK2-receptors, and/or neurokinin B, which which to NK3-receptors are involved. Due to the action which is advantageously directed at the peripheral region, the compounds of Formula I are suitable in particular for the treatment and/or inhibition of any pathology where either neurokinin A and/or NK2-receptors, or neurokinin B and/or NK3-receptors, or both neurokinin A and neurokinin B and/or NK2 and NK3-receptors are involved. Some compounds of the present invention, and particularly those in which R2 is a cyano-substituted naphthalene ring system, are also suitable for inhibiting processes in which tachykinins, such as substance P, which bind to NK1-receptors, are involved. Due to their action which is advantageously directed at the peripheral region, compounds of Formula I in which R2 is a cyano-substituted naphthalene ring system are suitable in particular for the treatment and/or inhibition of any pathology where substance P and/or NK1-receptors, or neurokinin A and/or NK2-receptors, or neurokinin B and/or NK3-receptors, or any combination of two or all three substance P, neurokinin A and neurokinin B and/or NK1, NK2 and NK3-receptors are involved.

The compounds of the present invention appear particularly suitable for the treatment and/or inhibition of pathologies of the respiratory, gastrointestinal, urinary, immune and cardiovascular system and of the central nervous system as well as pain, migraine, inflammation, nausea and vomiting, and skin diseases.

The compounds of the present invention also appear particularly suitable for the treatment and/or inhibition of pathologies of respiratory diseases, in particular asthma, chronic obstructive pulmonary disease, chronic obstructive bronchitis, bronchitis, cough, and rhinitis; skin diseases, in particular inflammatory skin reactions, allergic skin reactions, and psoriasis; arthropathy diseases, in particular arthritis, vasculitides and systemic lupus erythematosus; functional or inflammatory disorders in the gastrointestinal tract, in particular pseudomembranous colitis, gastritis, acute and chronic pancreatitis, ulcerative colitis, Crohn's disease and diarrhea; bladder diseases such as cystitis and interstitial cystitis; cardiovascular diseases such as hypertension, treatment of cancer especially melanomas, gliomas, small-cell and large-cell lung cancers, diseases of the immune system, bipolar disorders; migraine; pain, anxiety, depression, cognitive disorders, stress-related somatic disorders, psychosis, in particular schizophrenia, mania, schizoaffective disorder and panic disorders.

The functional disorders in the gastrointestinal tract which can be treated with the compounds of the invention include in particular the disorders of the lower intestinal tracts known under the name “irritable bowel syndrome” (=IBS). Typical symptoms for the diagnosis of IBS are described, for example, in W. G. Thompson et al., Gastroenterology International 2 (1989) 92-95 or in W. G. Thompson et al., GUT 45/II (1999) II43-II47, and are generally known among experts by the term “Rome Criteria”. The essential symptoms of IBS accordingly include pains in the lower abdomen, which appear to be due to hypersensitivity of the visceral afferent nervous system, and anomalies in bowel movement, such as constipation, diarrhea or alternating constipation and diarrhea. Further inflammatory disorders in the gastrointestinal tract which can be beneficially influenced by the group of compounds according to the invention are for example the inflammatory disorders in the small intestine and large intestine regions usually covered by the term “inflammatory bowel disease” (=IBD), for example ulcerative colitis or Crohn's disease. Owing to their action mechanism, the compounds according to the invention furthermore appear suitable for the treatment of other disorders in which tachykinins and in particular neurokinin A are involved as transfer agents. These disorders include for example neurogenic inflammation, inflammatory joint diseases such as rheumatic arthritis, asthmatic complaints, allergic disorders, disorders of immune regulation, bladder inflammation or also functional dyspepsia.

Other advantages of the compounds of the present invention are the synergistic effect between the NK2- and NK3-profiles and their very balanced combined NK2- and NK3-profile.

Description of the Pharmacological Test Methods

The example numbers given for the compounds of Formula I used as test substances in the pharmacological tests given below relate to the preparation examples described below.

1. Determination of the Binding Potency of the Test Substances to NK1-Receptors in Vitro

The affinity of test compounds for NK1-receptors can be determined by measuring the ability of the test compound to displace a radiolabeled ligand from its specific binding site. The tests were performed at Solvay Pharmaceuticals, Weesp, The Netherlands.

The radiolabel used in this assay is [³H]-Substance P. Receptors were obtained from membrane preparations of CHO-cells (Chinese Hamster Ovary cells), in which the human NK1-receptor was stably expressed. Membranes were incubated with [³H]-Substance P in the absence or presence of test compounds at different concentrations, diluted in a buffer system. Separation of bound radioactivity from free radioactivity was done by filtration through glass fiber filters (Packard GF/B) with several washings with ice-cold buffer solution. Bound radioactivity was measured with a liquid scintillation counter (total binding). Unspecific binding was determined by incubation with an excess (1 μmol/l) of unlabeled Substance P. Specific binding is obtained by subtracting the unspecific from the total binding.

Radioactivity of the specific binding was plotted against the concentration of the test compound and IC₅₀ values, i.e. the concentration of test compound by which 50% of the radioligand is displaced, were calculated. The inhibition constant (Ki) was calculated according to the Cheng-Prusoff equation, and listed as its negative logarithmic value (pKi). The pKi value describes the potency of a test compound to bind to a receptor.

TABLE 1
Binding potency of the test substances to NK1-receptors in vitro

| | Example No. | | | | | | | | |
|---|---|---|---|---|---|---|---|---|---|
| | 1 | 4 | 8 | 9 | 10 | 11 | 14 | 15 | 24 |
| pKi | 7.1 | 7.2 | 7.4 | 7.6 | 7.1 | 8.2 | 7.0 | 7.6 | 7.1 |
| | Example No. | | | | | | | | |
| | 49 | 55 | 56 | 65 | 70 | 80 | 82 | 83 | 89 |
| pKi | 7.0 | 7.0 | 7.2 | 7.1 | 7.2 | 7.1 | 7.1 | 7.1 | 7.0 |

| | Example No. | | | | | | | | |
|---|---|---|---|---|---|---|---|---|---|
| | 92 | 93 | 98 | 101 | 102 | 103 | 105 | 108 | 116 |
| pKi | 7.0 | 8.1 | 8.3 | 8.7 | 8.7 | 7.3 | 7.6 | 7.9 | 7.1 |

| | Example No. | | |
|---|---|---|---|
| | 125 | 126 | 127 |
| pKi | 7.9 | 8.2 | 7.1 |

For the compounds of all examples, the affinity to human NK1-receptors was determined in each case by at least three measurements of the test substances in concentration series of 10⁻⁶ to 10⁻¹⁰ mol/l. The average result of all measurements is listed above. The compounds of Example No. 1, 4, 8 to 11, 14, 15, 24, 49, 55, 56, 65, 70, 80, 82, 83, 89, 92, 93, 98, 101 to 103, 105, 108, 116, and 125 to 127 exhibited pKi values of at least 7.0 in this test model. The compounds of Example No. 11, 93, 98, 101, 102 and 126 exhibited pKi values of at least 8.0.

2. Determination of the Binding Potency of the Test Substances to NK2-Receptors in Vitro

The affinity of test compounds for NK2-receptors can be determined by measuring the ability of the test compound to displace a radiolabeled ligand from its specific binding site.

The radiolabel used in this assay is [³H]-SR 48968 (saredutant). Receptors were obtained from membrane preparations of CHO-cells (Chinese Hamster Ovary cells), in which the human NK2-receptor was stably expressed. Membranes were incubated with [³H]-saredutant in the absence or presence of test compounds at different concentrations, diluted in a buffer system. Separation of bound radioactivity from free radioactivity was done by filtration through glass fiber filters (Packard GF/B) with several washings with ice-cold buffer solution. Bound radioactivity was measured with a liquid scintillation counter (total binding). Unspecific binding was determined by incubation with an excess (0.1 μmol/l) of unlabeled saredutant. Specific binding is obtained by subtracting the unspecific binding from the total binding.

Radioactivity of the specific binding was plotted against the concentration of the test compound and IC₅₀ values, i.e. the concentration of test compound by which 50% of the radioligand is displaced, were calculated. The inhibition constant (Ki) was calculated according to the Cheng-Prusoff equation, and listed as its negative logarithmic value (pKi). The pKi value describes the potency of a test compound to bind to a receptor.

TABLE 2
Binding potency of the test substances to NK2-receptors in vitro

| | Example No. | | | | | | | | |
|---|---|---|---|---|---|---|---|---|---|
| | 1 | 2 | 3 | 4 | 5 | 6 | 7 | 8 | 9 |
| pKi | 9.8 | 9.4 | 8.8 | 9.6 | 9.0 | 9.2 | 8.7 | 9.1 | 9.2 |
| | Example No. | | | | | | | | |
| | 10 | 11 | 12 | 13 | 14 | 15 | 16 | 17 | 18 |
| pKi | 8.9 | 8.7 | 8.7 | 9.0 | 9.1 | 9.2 | 9.0 | 7.5 | 8.9 |
| | Example No. | | | | | | | | |
| | 19 | 20 | 21 | 22 | 23 | 24 | 25 | 26 | 27 |
| pKi | 8.5 | 7.6 | 7.9 | 7.8 | 8.7 | 8.3 | 8.3 | 8.3 | 7.8 |
| | Example No. | | | | | | | | |
| | 28 | 29 | 30 | 31 | 32 | 33 | 34 | 35 | 36 |
| pKi | 7.7 | 8.6 | 8.6 | 8.4 | 7.8 | 7.1 | 7.8 | 8.5 | 8.8 |
| | Example No. | | | | | | | | |
| | 37 | 38 | 39 | 40 | 41 | 42 | 43 | 44 | 45 |
| pKi | 8.8 | 8.8 | 7.7 | 7.9 | 8.5 | 7.8 | 9.6 | 8.3 | 8.6 |
| | Example No. | | | | | | | | |
| | 46 | 47 | 49 | 50 | 51 | 52 | 53 | 55 | 56 |
| pKi | 8.2 | 8.5 | 8.0 | 7.4 | 7.1 | 7.8 | 7.8 | 8.2 | 7.6 |
| | Example No. | | | | | | | | |
| | 58 | 59 | 61 | 63 | 64 | 65 | 66 | 67 | 68 |
| pKi | 7.0 | 7.0 | 7.4 | 8.4 | 7.9 | 7.6 | 7.8 | 8.2 | 8.7 |
| | Example No. | | | | | | | | |
| | 69 | 70 | 71 | 72 | 73 | 74 | 75 | 76 | 77 |
| pKi | 7.0 | 7.6 | 8.3 | 8.5 | 7.9 | 8.4 | 8.3 | 7.8 | 8.5 |
| | Example No. | | | | | | | | |
| | 78 | 79 | 80 | 81 | 82 | 83 | 84 | 85 | 86 |
| pKi | 8.8 | 8.7 | 8.7 | 7.9 | 8.6 | 8.3 | 7.9 | 7.9 | 7.9 |
| | Example No. | | | | | | | | |
| | 87 | 89 | 90 | 91 | 92 | 93 | 94 | 95 | 96 |
| pKi | 8.1 | 7.9 | 7.9 | 8.1 | 7.7 | 7.8 | 8.5 | 9.0 | 8.7 |
| | Example No. | | | | | | | | |
| | 97 | 98 | 99 | 100 | 101 | 102 | 103 | 104 | 105 |
| pKi | 8.7 | 8.3 | 9.2 | 8.5 | 9.4 | 8.3 | 8.7 | 8.7 | 8.7 |

Example No.

| | 108 | 116 | 124 | 125 | 126 | 127 |
|---|---|---|---|---|---|---|
| pKi | 8.4 | 8.3 | 9.0 | 7.3 | 8.0 | 8.7 |

For the compounds of Example Nos. 1 to 47, 49 to 53, 55, 56, 58, 59, 61, 63 to 104 and 124 to 127, the affinity to human NK2-receptors was determined in each case by at least three measurements of the test substances in concentration series of 10⁻⁶ to 10⁻¹⁰ mol/l. The average result of all measurements is listed above. All the aforementioned test substances exhibited pKi values of at least 7.0 in this test model. The compounds of Example No. 1 to 16, 18, 19, 23 to 26, 29 to 31, 35 to 38, 41, 43 to 47, 49, 55, 63, 67, 68, 71, 72, 74, 75, 77 to 80, 82, 83, 87, 91, 94 to 105, 108, 116, 124, 126 and 127 exhibited pKi values of at least 8.0. The compounds of Example No. 1, 2, 4 to 6, 8, 9, 13 to 16, 95, 104 and 124 exhibited pKi values of at least 9.0.

3. Determination of the Binding Potency of the Test Substances to NK3-Receptors in Vitro

The affinity of test compounds for NK3-receptors can be determined by measuring the ability of the test compound to displace a radiolabeled ligand from its specific binding site.

The radiolabel used in this assay is [³H]-SR 142801 (osanetant). Receptors were obtained from membrane preparations of CHO-cells (Chinese Hamster Ovary cells), in which the human NK3-receptor was stably expressed. Membranes were incubated with [³H]-osanetant in the absence or presence of test compounds at different concentrations, diluted in a buffer system. Separation of bound radioactivity from free radioactivity was done by filtration through glass fiber filters (Packard GF/B) with several washings with ice-cold buffer solution. Bound radioactivity was measured with a liquid scintillation counter (total binding). Unspecific binding was determined by incubation with an excess (1 μmol/l) of unlabeled osanetant. Specific binding is obtained by subtracting the unspecific binding from the total binding.

Radioactivity of the specific binding was plotted against the concentration of the test compound and IC₅₀ values, i.e. the concentration of test compound by which 50% of the radioligand is displaced, were calculated. The inhibition constant (Ki) was calculated according to the Cheng-Prusoff equation, and listed as its negative logarithmic value (pKi) The pKi value describes the potency of a test compound to bind to a receptor.

TABLE 3
Binding potency of the test substances to NK3-receptors in vitro

| | Example No. | | | | | | | | |
|---|---|---|---|---|---|---|---|---|---|
| | 1 | 3 | 4 | 5 | 6 | 7 | 9 | 10 | 11 |
| pKi | 8.6 | 8.2 | 8.2 | 8.3 | 8.7 | 8.0 | 8.4 | 8.0 | 7.9 |
| | Example No. | | | | | | | | |
| | 12 | 13 | 14 | 15 | 16 | 18 | 19 | 20 | 21 |
| pKi | 7.9 | 7.4 | 8.5 | 8.4 | 7.8 | 8.3 | 7.5 | 7.0 | 7.2 |
| | Example No. | | | | | | | | |
| | 22 | 23 | 24 | 25 | 26 | 28 | 29 | 30 | 31 |
| pKi | 7.3 | 8.0 | 7.0 | 7.2 | 7.3 | 7.0 | 7.3 | 7.7 | 7.4 |
| | Example No. | | | | | | | | |
| | 32 | 33 | 35 | 36 | 37 | 38 | 39 | 40 | 41 |
| pKi | 7.4 | 7.5 | 7.0 | 8.3 | 7.7 | 7.6 | 7.5 | 7.2 | 7.8 |
| | Example No. | | | | | | | | |
| | 43 | 44 | 45 | 46 | 47 | 49 | 50 | 51 | 52 |
| pKi | 8.3 | 7.5 | 7.9 | 7.5 | 7.7 | 8.1 | 8.0 | 8.2 | 7.9 |
| | Example No. | | | | | | | | |
| | 53 | 54 | 55 | 56 | 57 | 60 | 63 | 64 | 65 |
| pKi | 8.5 | 8.2 | 8.4 | 8.3 | 7.2 | 7.2 | 7.7 | 7.1 | 7.8 |
| | Example No. | | | | | | | | |
| | 66 | 67 | 68 | 69 | 70 | 71 | 72 | 73 | 74 |
| pKi | 7.7 | 8.3 | 8.0 | 7.3 | 7.2 | 7.4 | 7.4 | 7.4 | 7.6 |
| | Example No. | | | | | | | | |
| | 75 | 76 | 77 | 78 | 79 | 80 | 81 | 82 | 83 |
| pKi | 7.4 | 7.3 | 7.1 | 7.6 | 7.7 | 7.1 | 7.1 | 7.6 | 8.5 |
| | Example No. | | | | | | | | |
| | 84 | 85 | 86 | 87 | 89 | 90 | 91 | 92 | 95 |
| pKi | 7.4 | 7.3 | 7.2 | 7.4 | 7.8 | 7.1 | 7.8 | 7.0 | 7.7 |

| | Example No. | | | | | | | | | |
|---|---|---|---|---|---|---|---|---|---|---|
| | 97 | 98 | 99 | 101 | 102 | 103 | 104 | 124 | 125 | 126 |
| pKi | 7.1 | 7.2 | 7.2 | 7.7 | 7.0 | 7.0 | 7.7 | 7.8 | 7.3 | 7.4 |

For the compounds of Example Nos. 1, 3 to 7, 9 to 16, 18, 19, 21 to 23, 25, 26, 29 to 33, 36 to 41, 43 to 47, 49 to 57, 60, 63 to 87, 89 to 91, 95, 97 to 104, and 124 to 126 the affinity to human NK3-receptors was determined in each case by at least three measurements of the test substances in concentration series of 10⁻⁶ to 10⁻¹⁰ mole/liter. The average result of all measurements is listed above. All the aforementioned test substances exhibited pKi values of at least 7.0 in this test model. The compounds of Example Nos. 1, 3 to 10, 14, 15, 18, 23, 36, 43, 49 to 51, 53 to 56, 67, 68 and 83 exhibited pKi values of at least 8.0.

4. Functional Cellular Tests of the NK1-, NK2- and NK3-Antagonism

Functional cellular tests of the antagonistic effect of the compounds of the present invention on the human tachykinin receptors were performed in CHO cells expressing the recombinant human NK1, NK2 or NK3-receptor. In these tests the inhibition of ligand-induced increase in mobilization of intracellular calcium and inhibition of ligand-induced phosphorylation of mitogen activated protein kinase (MAPK) were determined, which can be used as a measure of functional activity of tachykinin-antagonists. Additionally, the antagonistic properties of reference compounds on the different tachykinin receptors were characterized for comparison.

The effects of test compounds were assessed using Chinese hamster ovary (CHO) fibroblast cells, stably expressing cloned human NK1, NK2 or NK3-receptors. The NK receptor is coupled to G_{q}. The activation of the G_{q} protein by ligand binding to the receptor leads to a mobilization of intracellular calcium and phosphorylation of MAPK. Both systems were used to determine functional effects of the test compounds.

Ca²⁺ Measurements Using FLIPR for NK1 and NK2 Activity

For tests, cells were seeded 24 hours prior to the experiment into black 96-well microplates. The cell density was 2.2×10⁴ cells/well. All steps were done under sterile conditions. In order to observe changes in intracellular calcium levels, cells were loaded with a calcium-sensitive dye. This dye (FLUO-4, from Molecular Probes) excites at 488 nm, and emits in the 500 nm to 560 nm range, only if a complex with calcium is formed. For the dye loading the growth-medium was aspirated out of the well without disturbing the confluent cell layer and 100 μl loading medium (HBSS, 4 μM FLUO-4, 0.005% (w/v) pluronic acid, 2.5 mM probenecid, 20 mM HEPES, pH 7.4) was dispensed into each well using an automatic pipettor system (Multidrop, Labsystems). Pluronic acid was added to increase dye solubility and dye uptake into the cells, whereas probenecid, an anion exchange inhibitor, was added to the loading medium to increase dye retention in the cells. The cells were incubated in a 5% CO₂ incubator at 37° C. for 40 minutes. After dye loading, the cells were washed three times with wash-buffer (HBSS, 2.5 mM probenecid, 20 mM HEPES, pH 7.4) to reduce basal fluorescence. In the last washing step the buffer was aspirated and replaced with 100 μl washing buffer. For the antagonism screening mode 50 μl of the compound (final concentration ranges from 10 μM to 1.4 nM) were applied 7 min prior to addition of substance P (final concentration: 10⁻⁸M; NK1 agonist) or NKA (final concentration: 10⁻⁷M; NK2 agonist). The FLIPR setup parameters were set to 0.4 sec exposure length, filter 1, 50 μl fluid addition, pipettor height at 125 μl, dispense speed 40 μl/sec without mixing. Maximal fluorescence changes were obtained using the statistic function of the FLIPR software, and data plotted using GraphPad Prism 4. All points were expressed as a percentage inhibition of the control agonist. IC₅₀ values were determined using sigmoidal dose-response curve fitting. Antagonist potencies (pA₂) values were calculated using equation:
pA₂=−log(IC₅₀/(1+[L]/EC₅₀)),

in which the IC₅₀ of the test compound was obtained from concentration-effect relationships, [L] is the concentration of the agonist (substance P for NK1 test, NKA for NK2 test, NKb for NK3 test), and the EC₅₀ is the potency of the agonist at the respective human cloned NK receptor (EC₅₀ substance P: 10^{-9.6}M; EC₅₀ NKA: 10^{-8.8}M, EC₅₀ NKB: 10^{-8.8}M). The results are summarized in tables 4 and 5:

TABLE 4
pA₂ data for NK1:

| | Example No. | | | | | | | | |
|---|---|---|---|---|---|---|---|---|---|
| | 1 | 8 | 9 | 10 | 11 | 13 | 14 | 15 | 23 |
| pA₂ | 8.1 | 7.1 | 8.0 | 7.1 | 7.9 | 7.5 | 7.3 | 7.2 | 8.9 |
| | Example No. | | | | | | | | |
| | 93 | 95 | 96 | 98 | 99 | 101 | 102 | 103 | 104 |
| pA₂ | 8.1 | 7.2 | 7.5 | 8.8 | 7.3 | 7.2 | 8.7 | 7.5 | 7.7 |

| | Example No. | | |
|---|---|---|---|
| | 105 | 108 | 116 |
| pA₂ | 7.5 | 7.5 | 7.5 |

TABLE 5
pA₂ data for NK2

| | Example No. | | | | | | | | |
|---|---|---|---|---|---|---|---|---|---|
| | 1 | 2 | 3 | 4 | 5 | 6 | 7 | 8 | 9 |
| pA₂ | 9.7 | 9.3 | 8.7 | 8.6 | 8.5 | 8.8 | 8.6 | 8.6 | 8.6 |
| | Example No. | | | | | | | | |
| | 10 | 11 | 12 | 13 | 14 | 15 | 16 | 17 | 18 |
| pA₂ | 8.4 | 8.4 | 8.3 | 8.3 | 8.2 | 8.3 | 8.3 | 7.8 | 8.5 |
| | Example No. | | | | | | | | |
| | 19 | 23 | 36 | 37 | 43 | 51 | 52 | 55 | 67 |
| pA₂ | 9.3 | 8.9 | 9.3 | 8.8 | 9.2 | 7.4 | 8.4 | 8.7 | 7.6 |
| | Example No. | | | | | | | | |
| | 68 | 83 | 93 | 94 | 95 | 96 | 97 | 98 | 99 |
| pA₂ | 8.0 | 7.8 | 8.4 | 8.8 | 8.5 | 8.9 | 9.6 | 9.3 | 9.6 |

| | Example No. | | | | | | |
|---|---|---|---|---|---|---|---|
| | 101 | 102 | 103 | 104 | 105 | 108 | 116 |
| pA₂ | 10.0 | 7.4 | 8.7 | 7.8 | 10.1 | 9.0 | 8.9 |

Ca²⁺ Measurements Using Aequorin for NK3 Activity

NK3 antagonism was measured in a cell line expressing the human NK3 receptor and mitochondrially targeted apoaequorin. In this system, cells expressing apoaequorin are incubated with coelenterazine, which is the chromophore co-factor of aequorin. Upon incubation of the cells with senktide, a potent agonist on NK3, intracellular calcium concentration increases. Traces of free calcium lead to a concentration dependent activation of the catalytic activity of aequorin, which oxidizes coelenterazine and yields apoaequorin, coelenteramide, CO2 and light (λmax=469 nm). Once the photon has been emitted, the complex must dissociate and the apoaequorin must recombine with the co-factor before the complex can emit another photon. Thus, in this system, measurements of luminescence (light emission) following senktide addition reflects an increase in intracellular calcium due to activation of NK3 receptors.

For the test, cells were grown to confluency and harvested with complexon (5 mM EDTA in PBS). Cells were centrifugated and resuspended in DMEM/F-12 (nutrient mixture according to Ham) without phenolred, supplemented with 10% FCS to a density of 5×106 cells/ml. In order to load the cells, coelenterazine was added to a final concentration of 5 μM and cells were stirred at room temperature for 4 hours. Loaded cells were diluted in DMEM/F12 without phenolred and supplements to a density of 2.8×105 cells/ml, pre-heated to 37° C., and stirred for 60 min at room temperature. For the antagonism screening mode 15 μl of the cell suspension were added to 85 μl of the compound (final concentration ranges from 4.5 nM to 10 μM) in white 96-well plates. After an incubation time of 20 min 50 μl senktide (final concentration of 5×10−8M) were applied and chemoluminescence was measured immediately for 20 seconds using the Microlumat (Berthold). All points were expressed as a percentage inhibition of the control agonist. IC50 values were determined using sigmoidal dose-response curve fitting of GraphPad Prism 4. Antagonist potencies (pA2) values were calculated using equation:
pA2=pIC50+log [(L/EC50)-1],

in which the pIC50 is the negative logarithm of the IC50 value of the test compound that was obtained from concentration-effect relationships, L is the concentration of senktide and EC50 its potency at the human cloned NK3 receptor (EC50 senktide: 10−8.8M).

TABLE 6
pA₂ data for NK3

| | Example No. | | | | | | | | |
|---|---|---|---|---|---|---|---|---|---|
| | 1 | 2 | 3 | 4 | 5 | 6 | 7 | 8 | 9 |
| pA₂ | 8.6 | 7.5 | 8.2 | 8.2 | 8.5 | 8.7 | 8.1 | 8.4 | 8.0 |

| | Example No. | | | | | | | | |
|---|---|---|---|---|---|---|---|---|---|
| | 10 | 11 | 12 | 13 | 14 | 15 | 16 | 17 | 18 |
| pA₂ | 8.3 | 8.1 | 7.9 | 8.0 | 8.1 | 8.1 | 7.9 | 6.9 | 8.0 |
| | Example No. | | | | | | | | |
| | 19 | 23 | 36 | 37 | 43 | 51 | 53 | 55 | 67 |
| pA₂ | 7.3 | 7.9 | 7.2 | 7.5 | 7.5 | 7.5 | 7.3 | 8.1 | 7.8 |

Example No.

| | 97 | 101 | 102 | 103 | 104 | 105 |
|---|---|---|---|---|---|---|
| pA₂ | 7.1 | 7.9 | 7.4 | 7.3 | 7.8 | 7.0 |

5. Determination of Functional NK1-Receptor Antagonism of Test Compounds in Tissue Isolated from Guinea Pigs.

The NK1-antagonistic action of test compounds was tested in aortic ring preparations isolated from guinea pigs. The preparations were kept in an oxygenated nutrient solution kept at 37° C. For measuring contraction of the circular vascular muscle, the preparations were fixed to a hook and connected to force displacement transducers. Contractions/relaxations were recorded on a pen recorder. The preparations were given a medium tonus by addition of phenylephrine.

The NK1-receptors were stimulated with the NK1-receptor agonist Substance P, causing a relaxation of the muscular tone. Before (predrug) and after the administration of the test compound such relaxations were measured and quantified in percent of the predrug relaxation. 2-3 concentrations of the test compound were applied showing the inhibition of the receptor stimulation concentration dependently. The concentration of half-maximal inhibition (IC₅₀) and its negative logarithm pIC₅₀=−log(IC₅₀) was calculated. The pIC₅₀ value indicates the inhibitory potency of the test compounds to the NK1-receptor.

TABLE 7
Functional NK1-antagonism of the test substances
on isolated guinea pig tissue

| | Example No. |
|---|---|
| | 11 |
| pIC50 | 8.26 |

6. Determination of Functional NK2-Receptor Antagonism of Test Compounds in Tissue Isolated from Guinea Pigs.

The NK2 antagonistic action of test compounds was tested in gall bladder preparations isolated from guinea pigs. The preparations were kept in an oxygenated nutrient solution kept at 37° C. For measuring contraction of the gall bladder muscle, the preparations were fixed to a hook and connected to force displacement transducers. Contractions were recorded on a pen recorder.

The NK2-receptors were stimulated with the NK2-receptor agonist neurokinin A, causing a contraction of the muscle. Before (predrug) and after the administration of the test compound such contractions were measured and quantified in percent of the predrug contraction. 2-3 concentrations of the test compound were applied showing the inhibition of the receptor stimulation concentration dependently. The concentration of half-maximal inhibition (IC₅₀) and its negative logarithm pIC₅₀=−log(IC₅₀) was calculated. The pIC₅₀ value indicates the inhibitory potency of the test compounds to the NK2-receptor.

TABLE 8
NK-2-receptor-antagonistic effectiveness of the test substances of
Formula I on the guinea pig gall bladder in vitro

| | Example No. | | | | | | | | |
|---|---|---|---|---|---|---|---|---|---|
| | 1 | 2 | 3 | 4 | 6 | 10 | 11 | 36 | 43 |
| pA2 | 8.17 | 8.96 | 8.96 | 8.86 | 8.88 | 8.78 | 8.03 | 9.05 | 10.41 |

7. Determination of Functional NK3-Receptor Antagonism of Test Compounds in Tissue Isolated from Guinea Pigs.

The NK3-antagonistic action of test compounds was tested in ileal preparations isolated from guinea pigs. The preparations were kept in an oxygenated nutrient solution kept at 37° C. For measuring contraction of the longitudinal muscle of the ileum, the preparations were fixed to a hook and connected to force displacement transducers. Contractions were recorded on a pen recorder.

The NK3-receptors were stimulated with the NK3-receptor agonist [MePhe⁷]-neurokinin B, causing a contraction of the muscle. Before (predrug) and after the administration of the test compound such contractions were measured and quantified in percent of the predrug contraction. 2-3 concentrations of the test compound were applied showing the inhibition of the receptor stimulation concentration dependently. The concentration of half-maximal inhibition (IC₅₀) and its negative logarithm pIC₅₀=−log(IC₅₀) was calculated. The pIC₅₀ value indicates the inhibitory potency of the test compounds to the NK3-receptor.

TABLE 9
Functional NK3-antagonism of the test substances
on isolated guinea pig tissue

| | Example No. | | |
|---|---|---|---|
| | 1 | 3 | 4 |
| pA2 | 8.4 | 8.5 | 8.0 |

8. Determination of the NK3-Receptor-Antagonistic Effectiveness of the Test Substances In Vivo (Reduction of Body Temperature in Gerbils with Senktide-Induced Hypothermia)

NK3 agonists reduce the body temperature of gerbils. The senktide-induced hypothermia can be antagonized by administering compounds with NK3-antagonistic properties. Measuring the level of hypothermia is indicative for the degree of activity of the test compounds. The own effect of the tested compound is assessed in the same experiment to exclude an additional hyperthermic effect.

Male gerbils with a body weight between 60 to 90 g are housed in groups under normal day-night rhythm and under constant environmental temperature (room temperature: 21±2° C.) with a constant relative humidity level (50±10%). Water and food are freely available. Reference agonist: Senktide 0.03 mg/kg sc. Antagonists: see example compounds in table X.

Per series of experiments, one condition is always the vehicle/vehicle group to determine the normal body temperature and one condition is always the vehicle/senktide group to determine the senktide induced hypothermia (=100%). The animals are weighted and marked 60 minutes prior to the agonist administration. For oral (po) testings, the test compounds are administered at t=−60 minutes. For parenteral or subcutaneous (i por sc, resp.) administration, at t=−30 minutes. At t=0 minutes the agonist Senktide is administered (sc). At t=15 minutes the temperature is measured orally, and registered with a 0.1° C. accuracy after a 10 second reading. This way, animals are measured every 60 sec.

Using the Dunett's test, the vehicle/vehicle group is used as reference for the analysis of the own effect; whereas the vehicle/senktide group is used for comparison for the interaction test (i.e. example compounds/senktide groups).

TABLE 10
Inhibition of temperature increase at t = 60 minutes
after oral (po) administration

| Example No. | Dose: 30 mg/kg | Dose: 10 mg/kg |
|---|---|---|
| 1 | 54% | 37% |

TABLE 11
Inhibition of temperature increase at a dose of 10 mg/kg
after parenteral (ip) administration

| | t = 15 minutes after | t = 30 minutes after |
|---|---|---|
| Example No. | administration | administration |
| 4 | 54% | 68% |
| 6 | — | 75% |

9. Determination of the NK-3-Receptor-Antagonistic Effectiveness of the Test Substances In Vivo (Reduction of Blood Pressure in Guinea-Pigs with Senktide-Induced Hypertension)

NK3 agonists increase the blood pressure in guinea pigs. The senktide-induced hypertension can be antagonized by administering compounds with NK3-antagonistic properties. Measuring the level of hypertension is indicative of the degree of activity of the test compounds. The effect of the tested compound is assessed in the same experiment to exclude an additional hypertensional effect.

Male Dunkin Hartley guinea pigs with a body weight between 450 and 550 g were anesthetized with Ketamin (200 mg/kg i.m.; Aescoket 10%) and the left carotid artery and left jugular vein were cannulated for blood pressure measurement and drug application, respectively. The animals were allowed to breathe spontaneously. Blood pressure was measured with a strain gauge transducer connected to a computer via an amplifier.

After hemodynamic stabilization two subsequent injections of the NK3-receptor agonist senktide (0.4 μg/kg i.v.; 0.5 ml/kg) were given with an interval of 15 minutes and the peak increase in mean arterial blood pressure of each injection determined. The mean value of the increase in blood pressure served as the pre-drug hypertensive effect of senktide. Five minutes thereafter the test compound was given as an infusion over 10 min (0.1 ml/min), immediately followed by an injection of 0.4 μg/kg senktide and the peak increase in mean arterial blood pressure determined. Up to 5 cycles of additive test compound dosages were applied and the hypertensive effect of senktide determined after each dose. The effect of the test compound on the peak rise in blood pressure induced by senktide is expressed as percentage relative to the pre-drug value. ID₅₀ values (dose that produces a 50% inhibition of the senktide response) were calculated from the dose response curve. The effect of the vehicle on the senktide induced pressure response was determined on regular base.

For oral administration, vehicle (1% methylcellulose) or test compound was administered in a dose volume of 5 ml/kg. Forty five minutes thereafter, anesthesia was induced and the carotid artery and jugular vein were cannulated. After a 10 minutes period for hemodynamic stabilization, two subsequent injections of the NK3-receptor agonist senktide (0.4 μg/kg i.v.; 0.5 ml/kg) were given with an interval of 15 minutes and the peak increase in mean arterial blood pressure of each injection determined. The first senktide injection was approximately 80 to 90 minutes post dosing. The mean value of the increase in blood pressure served as the hypertensive effect of senktide in that animal. The mean hypertensive effect of senktide following vehicle treatment served as the control value (100%). The effect of each test compound dose on the peak rise in blood pressure induced by senktide is expressed as percentage relative to this control value and averaged per dose group. ID₅₀ values (dose that produces a 50% inhibition of the senktide response) were calculated from the dose response curve.

Senktide (0.8 μg/ml) was dissolved in saline. For intravenous administration, the test compounds were dissolved in 40% hydroxypropyl-β-cyclodextrine (HPCD) containing 10% DMSO, diluted with saline and administered intravenously in cumulative dose ranges. A vehicle group of animals received the corresponding HPCD/DMSO solutions instead of the test compound. For oral administration, the test compound was suspended in 1% methylcellulose. A vehicle group of animals received the corresponding HPCD/DMSO solution instead of the test compound.

TABLE 12
Inhibition of blood pressure as ID₅₀-value after
intravenous (iv) and oral (po) administration

| | ID₅₀ (iv) | ID₅₀ (po) |
|---|---|---|
| Example No. | [mg/kg] | [mg/kg] |
| 1 | 0.2557 | 6.3796 |
| 2 | 1.9809 | — |
| 4 | 0.7564 | 2.3313 |
| 6 | 0.157 | — |
| 11 | 1.0554 | 10 |
| 13 | 0.5013 | <10 |
| 14 | 0.5168 | 6.811 |
| 15 | 0.3732 | — |
| 18 | 1.0108 | — |
| 19 | 0.3902 | — |

10. Determination of the NK1- and NK2-Receptor-Antagonistic Effectiveness of the Test Substances In Vivo

The NK1- and NK2-antagonistic activities of the test substances were investigated in anaesthetised guinea pigs in each case after intravenous (=i.v.) and oral (=p.o.) administration in vivo. With the present test model it is possible to detect both NK2-antagonistic effects in three different organ systems (respiratory tracts, colon and circulation) and NK1-antagonistic effects (rapid drop in blood pressure) in an animal simultaneously.

Pirbright-White guinea pigs of a body weight of 500-700 g were anaesthetised with ketamine/xylazine (67/13 mg/kg subcutaneously, initial dose, further doses administered as required). The animals were provided with an intravenous catheter in order to administer the substance and an intra-arterial catheter to measure the blood pressure. The animals were artificially ventilated via a tracheal cannula and the respiratory pressure was recorded by means of a pressure transducer. A balloon was introduced into the distal colon of the animals for manometric recording of colon motility by means of a pressure transducer. Blood pressure, heart rate, respiratory pressure and colonic pressure were measured continuously for each animal and plotted on a recorder and by means of a digital data-processing system. Neurokinin A (=NKA; 200 pmol/animal) was administered i.v. as a bolus as a test stimulus to stimulate the NK1- and the NK2-receptors. NKA injection resulted in an increase in respiratory pressure (bronchoconstriction) and colonic pressure, and in a biphasic drop in blood pressure. The first phase of hypotension (=phase of maximum hypotension within the first minute after administration of NKA) is mediated via NK-1 receptors, since they can be blocked completely by specific NK-1 receptor antagonists. The second phase of delayed hypotension (=phase of maximum hypotension after 2-5 min.) on the other hand is mediated via NK2-receptors, since they can be blocked by specific NK2-receptor antagonists. The doses of the test substances are given as ED₅₀ values which each result in a response to the NKA test stimulus which is reduced to 50% of the initial value, as characteristic variables for the individual measurement parameters bronchoconstriction, colonic pressure and change in blood pressure mediated by NK1 or NK2.

The antagonistic effects of the test substances were first investigated in cumulative form, the time of the NKA test stimulus being 1 min after the administration of the respective doses of the test substances had ended. These ED₅₀ values obtained from cumulative dose effect curves are plotted in Table 13.

TABLE 13
NK1- and NK2-receptor-antagonistic potency of the test substances of
Formula I on guinea pigs in vivo after intravenous administration

| | ED₅₀ iv [μmol/kg] after 1 min (cumulative) | | | |
|---|---|---|---|---|
| | NK1 | NK2 | NK2 | |
| Example | (early | (late | (broncho- | NK2 |
| No. | hypotension) | hypotension) | constriction) | (colonic motility) |
| 1 | >0.46 | 0.044 | 0.033 | <0.003 |
| 3 | >1.462 | 0.397 | 0.221 | 0.07 |
| 4 | >1.462 | 0.04 | 0.058 | 0.053 |
| 6 | >1.462 | 0.04 | 0.06 | 0.096 |
| 8 | >1.462 | 1.113 | 0.731 | 0.294 |
| 9 | >1.462 | 0.108 | 0.058 | 0.053 |
| 11 | 0.433 | 0.212 | 0.052 | 0.085 |
| 14 | >1.462 | 0.247 | 0.26 | 0.245 |
| 15 | >1.462 | 0.1 | 0.075 | 0.066 |
| 16 | >1.462 | 0.34 | 0.38 | 0.132 |
| 18 | >1.462 | 0.181 | 0.18 | 0.068 |
| 19 | >1.462 | 0.209 | 0.093 | 0.028 |

The measured values plotted in Table 13 above show, inter alia, that the compounds of Example No. 11 after cumulative administration i.v. (detection of the antagonism 1 min. after the administration of test substance had ended) caused a marked NK-1-receptor-antagonistic activity on the early hypotension. The data show further that compounds of all Example Nos. caused a marked NK2-receptor-antagonistic activity of colon motility, late drop in blood pressure and respiratory resistance.

In order additionally to detect the variation over time of the antagonistic effects of the test substances, the action of the NKA test stimulus was determined at different times (1, 30, 60, 90, 120, 150 and 180 min.) after oral administration of the test substances. The antagonistic effects of the test substances were then determined as “area under the curve” (“AUC”) over the investigation period after administration of the test substances (1-180 min after administration) and the ED₅₀ values after oral administration obtained therefrom were plotted in table 14.

TABLE 14
NK1- and NK-2-receptor-antagonistic potency of the test substances
of Formula I on guinea pigs in vivo after oral administration

| | ED₅₀ AUC_{1-180 min} oral [μmol/kg] | | | |
|---|---|---|---|---|
| | NK1 | NK2 | NK2 | |
| Example | (early | (late | (broncho- | NK2 |
| compound | hypotension) | hypotension) | constriction) | (colonic motility) |
| 1 | >3.2 | 2.236 | 0.457 | 1.08 |
| 4 | >10 | 12 | <3.2 | <3.2 |
| 8 | >3.2 | >3.2 | >3.2 | >3.2 |
| 11 | 23.027 | 2.509 | 2.213 | 11.228 |

The compounds according to the invention, in particular the compounds of Example No. 1, 4, 8 and 11 as shown in table 15, are furthermore active orally on the NK2. Example 11 was also active as NK1 receptor antagonists.

The compounds of Formula I may be administered in conventional pharmaceutical preparations. The doses to be used may vary individually and will naturally vary according to the type of condition to be treated and the substance used. In general, however, medicinal forms with an active substance content of 0.2 to 200 mg, in particular 1 to 50 mg, active substance per individual dose are suitable for administration to humans and larger mammals. The compounds may be contained according to the invention, together with conventional pharmaceutical auxiliaries and/or carriers, in solid or liquid pharmaceutical preparations. Examples of solid preparations are preparations which can be administered orally, such as tablets, coated tablets, capsules, powders or granules, or alternatively suppositories. These preparations may contain conventional pharmaceutical inorganic and/or organic carriers, such as talcum, lactose or starch, in addition to conventional pharmaceutical auxiliaries, for example lubricants or tablet disintegrating agents. Liquid preparations such as suspensions or emulsions of the active substances may contain the usual diluents such as water, oils and/or suspension agents such as polyethylene glycols and the like. Other auxiliaries may additionally be added, such as preservatives, taste correctives and the like.

The active substances may be mixed and formulated with the pharmaceutical auxiliaries and/or carriers in known manner. For the production of solid medicament forms, the active substances may for example be mixed with the auxiliaries and/or carriers in conventional manner and may be wet or dry granulated. The granules or powder can be poured directly into capsules or be pressed into tablet cores in conventional manner. These may be coated in known manner if desired.

The following examples are intended to illustrate the invention in further detail without limiting its scope.

The LC-MS data (API) were obtained by the following conditions:

Instrument-Description: API 100 Single Quad, PE Applied Biosystems

PE 200 BI LC-Pump, PE Applied Biosystems
Gilson XL 215 Autosampler, Gilson Inc.
Sedex 75 Lightscattering-Detector, S.E.D.E.R.E
   Column: XTerra MS C18, 2.5 um, 50×4.6 mm
   Guard-Column: XTerra MS C18, 3.5 um, 20×3.9 mm
   Solvent A: 0.01 M NH4ac pH 5.0+5% Acetonitrile
   Solvent B: Acetonitrile
   Gradientprofile: 100% A ----10 min---> 95% B
2 min isocratic 95% B
100% A <---0.5 min---- 95% B
100% A 2.5 min isocratic
   Flow: 1.0 ml/min
   Wavelength: 225 nm

The LC-MS data (ZQ) were obtained under the following conditions:

Instrument-Description: ZQ Single Quad, Waters/Micromass

Alliance HT 2795, Waters
PL-ELS 1000 Lightscattering-Detector, Polymer Labs
   Column: XTerra MS C18, 2.5 um, 50×4.6 mm
   Guard-Column: XTerra MS C18, 3.5 um, 10×2.1 mm
   Solvent A: 0.01 M NH4ac pH 5.0+5% Acetonitrile
   Solvent B: Acetonitrile
   Gradientprofile: 100% A 1 min isocratic
100% A ----6 min---> 95% B
1 min isocratic 95% B
100% A <---1 min---- 95% B
100% A 2 min isocratic
   Flow: 1.0 ml/min
   Wavelength: 205-350 nm

### EXAMPLE 1

### Synthesis of N-[(2S)-2-(3,4-dichlorophenyl)-4-oxobutyl]-N-methyl-benzamide as starting material for the preparation of example compounds 1, 2 and 99

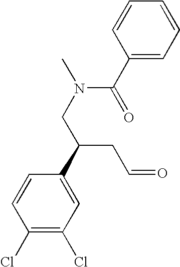

In a solution of 15 ml (0.172 mole) of oxalyl chloride in 200 ml of methylene chloride cooled to −60/−70° C. under nitrogen were added in 75 minutes dropwise in order to maintain the temperature at −60/−70° C. 30 ml of DMSO in 100 ml methylene chloride. The reaction mixture was stirred for 20 minutes at −70° C. and subsequently a suspension of 31 g (0.088 mole) of N-[2-(3,4-Dichlorophenyl)-4-hydroxy-butyl]-N-methyl-benzamide in 200 ml of methylene chloride were added in 60 minutes in a way to maintain the temperature of the reaction mixture at −60° C. As this temperature were added dropwise 90 ml of triethylamine in 40 ml methylene chloride. The mixture was let to come back to room temperature slowly and allowed to stay for 15 hours. The reaction mixture was concentrated in vacuum and the residue was dissolved in a mixture of 200 ml toluene and 200 ml ethyl acetate. The organic phase washed by 100 ml of a saturated solution of NaCl in water and 4 times with 100 ml water each. The recovered organic phase was dried on sodium sulfate and concentrated in vacuum to deliver 32.6 g of a glassy material.

17.15 g of the compound were put in suspension in 10 ml methylene chloride under stirring at room temperature. To the suspension were added 50 ml of n-hexane and the mixture was put at 45° C. giving a slurry. To the slurry were added 40 ml of n-hexane and the mixture was let to cool down to room temperature. The obtained suspension was filtered off to give a solid which washed three times with 10 ml n-hexane. After dissolution in methylene chloride and subsequent concentration to dryness 15 g of a solid (melting point: 78-79° C.) were obtained: Overall yield: 90%

### EXAMPLE 2

### Synthesis of 1′-[(3S)-4-[benzoyl(methyl)amino]-3-(3,4-dichlorophenyl)-butyl]-N,N-dimethyl-1,4′-bipiperidine-4′-carboxamide (example compound 1)

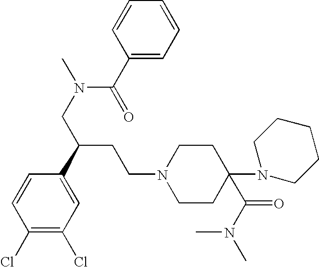

To a suspension of 15 g (0.0428 mole) of the aldehyde from example 1, 14.9 g (0.0540 mole) of [1,4′]bipiperidinyl-4′-carboxylic acid dimethylamide hydrochloride and 5.3 g sodium acetate in 800 ml of THF at room temperature under stirring were added 5 ml of acetic acid and the mixture was stirred for 5 hours at room temperature. To the mixture were added portion wise 18.5 g (0.0876 mole) of sodium triacetoxy-borohydride and the mixture was further stirred at room temperature for 15 hours. The mixture was concentrated to dryness in vacuum and re-dissolved in 300 ml ethyl acetate, 50 ml of MTBE and 6 g of KOH dissolved in 100 ml of water. The organic phase washed with sodium hydrogenocarbonate until pH 5-6 and 3 times with 100 ml of water. The organic solution was dried on sodium sulfate and concentrated to dryness to give 27 g of the raw base as a foam. 17 g of raw base were dissolved in 30 ml of methylene chloride at room temperature to which 13 ml of 5N HCl in isopropanol were added and a solution was obtained. To the solution were added 500 ml of MTBE and a solid appeared. The suspension was heated to 50° C. and cooled down. The solid was recovered by filtration, washed 3 times with 100 ml of MTBE. The solid was dissolved in 100 ml methanol and concentrated to dryness to give 16.2 g of the compound identified as dihydrochloride in elemental analyses. Yield: 90%. Optical rotation: −16.3° (c=1% in methanol). Raw base purified by dissolution in water in the presence of concentrated HCl gives after elimination of insoluble by-products by extraction with MTBE and basification by means of KOH of the water phase and subsequent extraction of the base using methylene chloride a pure base as a yellowish compound with a 90%-yield. Optical rotation; −17.8° (c=1% in methanol). LC-MS: M+1 (monoisotope) 573. Retention time: 7.90 min (API); 5.38 min (ZQ). ¹HNMR (as base) (500 MHz, CDCl₃, 30° C.) δ: 7.40-7.25 (m, 4.6H), 7.25-7.0 (m, 2.7H), 6.9, 6.7 (2×bs, 0.7H), 3.82 (bs, 0.7H), 3.55 (dd, 0.7H), 3.5-3.25 (m, 4.4H), 3.2 (bs, 0.7H), 3.0 (m, 2H), 2.95-2.90 (2×s, 3H), 2.8-2.5 (m, 4.5H), 2.4-1.6 (m, 12H), 1.5 (s, 4H), 1,4 (s, 2H).

¹³CNMR (125 MHz, CDCl₃, 30° C.) δ: 173.5, 171.6, 143.0, 136.4, 132.4, 130.5, 128.4, 126.8, 66.9, 57.1, 56.1, 53.3, 51.7, 47.3, 41.7, 38.7, 37.7, 33.6, 30.9, 26.9, 25.2.

### EXAMPLE 3

### Synthesis of 1′-[(3S)-4-[benzoyl(methyl)amino]-3-(3,4-dichlorophenyl)-butyl]-N-methyl-1, 4′-bipiperidine-4′-carboxamide (example compound 2)

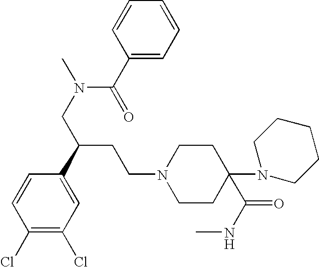

To a suspension of 15 g (0.0428 mole) of the aldehyde from example 1, 14.1 g (0.0540 mole) of [1,4′]bipiperidinyl-4′-carboxylic acid methylamide hydrochloride and, 5.3 g sodium acetate in 800 ml of THF at room temperature under stirring were added 5 ml of acetic acid and the mixture was stirred for 5 hours at room temperature. To the mixture were added portion wise 18.5 g (0.0876 mole) of sodium triacetoxyboro-hydride and the mixture was further stirred at room temperature for 15 hours. The mixture was concentrated to dryness in vacuum and re-dissolved in 300 ml ethyl acetate, 50 ml of MTBE and 6 g of KOH dissolved in 100 ml of water. The organic phase washed with sodium hydrogenocarbonate until pH 5-6 and 3 times with 100 ml of water. The organic solution was dried on sodium sulfate and concentrated to dryness to give 27 g of the raw base as an foam. 17 g of raw base were dissolved in 30 ml of methylene chloride at room temperature to which 13 ml of 5N HCl in isopropanol were added and a solution was obtained. To the solution were added 500 ml of MTBE and a solid appeared. The suspension was heated to 50° C. and cooled down. The solid was recovered by filtration, washed 3 times with 100 ml of MTBE. The solid was dissolved in 100 ml methanol and concentrated to dryness to give 16.2 g of the compound identified as dihydrochloride in elemental analyses. Yield: 100%. LC-MS: M+1: 559. Retention time: 6.98 (API)

¹HNMR (as base) (500 MHz, CDCl₃) δ: 7.45-6.70. (m, 9H), 3.84 (bs, 0.7H), 3.55-2.8 (m, 4.3H), 2.78-2.75 (2×s, 3H), 2.7-2.5 (m, 3H), 2.4-1.6 (m, 14H), 1.5 (s, 4H), 1,4 (s, 2H).

¹³CNMR (125 MHz, CDCl₃, 30° C.) δ: 176.9, 171.6, 143.0, 136.4, 132.4, 130.5, 128.3, 126.7, 64.6, 57.2, 56.0, 53.4, 50.8, 47.3, 41.7, 38.7, 34.2, 30.4, 29.5, 27.2, 25.8, 24.9

### EXAMPLE 4

### Synthesis of 1-[(3S)-4-[benzoyl(methyl)amino]-3-(3,4-dichlorophenyl)-butyl]-N,N-dimethyl-4-pyrrolidin-1-ylpiperidine-4-carboxamide (example compound 99)

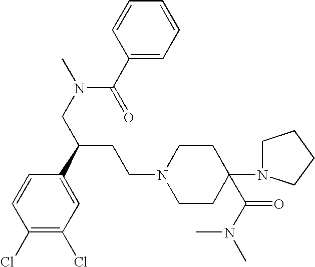

### Here: synthesis of N,N-dimethyl-4-pyrrolidin-1-ylpiperidine-4-carboxamide as starting material

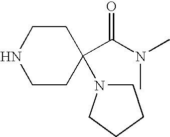

### Ethyl 1-benzyl-4-[(4-chlorobutanoyl)amino]piperidine-4-carboxylate

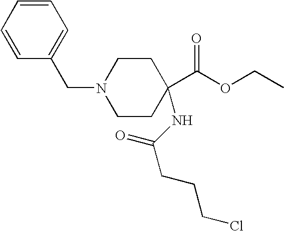

78.3 g (0.298 mole) ethyl 4-amino-1-benzylpiperidine-4-carboxylate and 50 ml pyridine (0.6 mol, 2 eq) were dissolved in 1 L CH₂Cl₂. The mixture was cooled to 5° C. and 0.37 ml chlorobutyryl chloride (0.33 mol, 1.1 eq) dissolved in 200 ml CH₂Cl₂ was added drop wise. The mixture was stirred over the weekend at ambient temperature. 800 ml of a saturated solution of NaHCO₃ were added and the organic layer was separated. The water layer was extracted with CH₂Cl₂ (2×500 ml). The organic layer washed with NaHCO₃ (2×800 ml) and evaporated to dryness. The mixture was purified over Silica (eluent: CH₂Cl₂/MeOH, 100/0 to 97/3 to 95/5, v/v). Yield: 113 g (˜quantitative yield) of a yellow semi solid.

### Ethyl 1-benzyl-4-(2-oxopyrrolidin-1-yl)piperidine-4-carboxylate

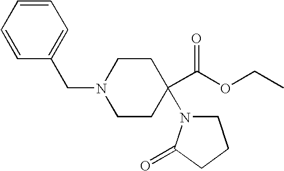

12 g sodium hydride (290 mmol, 1.2 eq) were suspended in 600 ml THF. To this suspension was added a gel of 88.5 g of ethyl 1-benzyl-4-[(4-chlorobutanoyl)-amino]-piperidine-4-carboxylate (240 mmol, 1 eq) in 200 ml THF. The mixture was stirred overnight at room temperature. NMR analysis revealed 60-70% conversion. 5 g of extra sodium hydride (120 mmol, 0.5 eq) and 9 g (60 mmol, 0.3 eq.) sodium iodide were added and the mixture was stirred for another day at room temperature. NMR analysis revealed now complete conversion. The reaction was quenched with 1 L of water. The mixture was extracted with ethyl acetate (3×1 L). The organic layer washed with 500 ml of a saturated solution of sodium chloride in water. The combined water layers were extracted with dichloromethane (2×1 L). The combined organic layers were evaporated to dryness. The crude mixture was further purified over Silica (eluent: CH₂Cl₂/MeOH, 100/0 to 95/5, v/v) yielding 68.4 g of the target compound (207 mmol, 67%) as light yellow solid.

### 1-Benzyl-N,N-dimethyl-4-(2-oxopyrrolidin-1-yl)piperidine-4-carboxamide

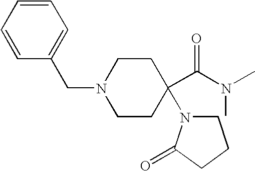

65 g (200 mmol, 1 eq) of ethyl 1-benzyl-4-(2-oxopyrrolidin-1-yl)piperidine-4-carboxylate was dissolved in 600 ml THF and 400 ml 1 M NaOH was added. The mixture was stirred at reflux overnight. TLC analysis revealed not complete conversion. 100 ml 1M extra NaOH was added and the mixture was stirred at reflux for another 2.5 h. The mixture was cooled to ambient temperature and diluted with 500 ml of water. The mixture was extracted with CH₂Cl₂ (2×500 ml). The water layer was cooled and neutralized with concentrated HCl (ca. 20 ml). The mixture was concentrated in vacuum to dryness.

This crude mixture was suspended in 1 L dichloromethane). Dimethylamine HCl salt (21.9 g, 270 mmol, 1.35 eq), TBTU (86.7 g, 270 mmol, 1.35 eq) and triethyl amine (138 ml, 1.0 mol, 5 eq) were added. The mixture was stirred overnight at room temperature. The mixture was diluted with CH₂Cl₂ (500 ml) and washed with water (500 ml), NaHCO₃ (500 mL), water and a saturated solution of sodium chloride in water (250 ml). The mixture was evaporated to dryness. The crude mixture was purified over Silica (eluent: CH₂Cl₂/MeOH, 95/5, v/v). Yield: 30.8 g of 1-benzyl-N,N-dimethyl-4-(2-oxopyrrolidin-1-yl)piperidine-4-carboxamide (93.5 mmol, 52%) as light yellow solid.

### N,N-Dimethyl-4-(2-oxopyrrolidin-1-yl)piperidine-4-carboxamide

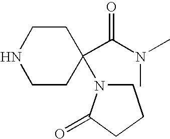

Benzyl-N,N-dimethyl-4-(2-oxopyrrolidin-1-yl)piperidine-4-carboxamide (29 g, 88 mmole) was dissolved in tert.-butanol/water (600 ml, 9/1, v/v). Pd—C (6 g, 10%, wet) was added and the mixture was stirred in hydrogen atmosphere (5 bar) at 50° C. for 16 h. NMR analysis revealed complete conversion. The Pd—C was removed by filtration over celite. The celite crop washed with tert.-butanol/water (100 mL, 9/1, v/v). The volatiles were removed by evaporation in vacuum. The mixture was further purified over Silica (eluent: CH₂Cl₂/3 N ammonia in methanol, 95/5 to 90/10, v/v). The appropriate fractions were pooled and concentrated to dryness. Yield: 18 g (75 mmol, 86%) as white solid.

### N,N-Dimethyl-4-pyrrolidin-1-ylpiperidine-4-carboxamide

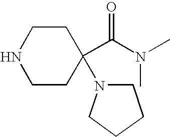

To the suspension of 1 g (4.2 mmole) N,N-dimethyl-4-(2-oxopyrrolidin-1-yl)-piperidine-4-carboxamide in the 50 ml absolute tetrahydrofuran 1.8 ml Redal (sodium-bis-(2-methoxyethoxy)-dihydroaluminate) (3.5M/toluene, 6.3 mmole) was added dropwise. The resulting solution was vigorously stirred for 4 hours at the room temperature, then reaction was quenched by the addition of 20 g NaF followed by 5 ml of 80% aqueous tetrahydrofuran solution. The resulting slurry was stirred for another hour, then solids were filtered out, gently washed with 2×50 ml tetrahydrofuran and the organic phase was evaporated under reduced pressure. Crude amine was further purified by column chromatography over silica gel (acetonitrile 6.25:aqueous ammonia 25% 1) delivering pale yellow oil with slowly crystallizes on standing (330 mg, 35%). Melting point: 150° C.

### Synthesis of 1-[(3S)-4-[benzoyl(methyl)amino]-3-(3,4-dichlorophenyl)-butyl]-N,N-dimethyl-4-pyrrolidin-1-ylpiperidine-4-carboxamide (example compound 99)

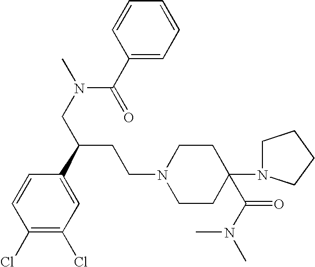

To a suspension of 150 mg (0.0428 mmole) of the aldehyde from example 1, 96 mg (0.0428 mmole) of N,N-dimethyl-4-pyrrolidin-1-ylpiperidine-4-carboxamide and 0.5 g sodium acetate in 21 ml of THF at room temperature under stirring were added 0.05 ml of acetic acid and the mixture was stirred for 5 hours at room temperature. To the mixture were added portion wise 0.13 g (0.062 mmole) of sodium triacetoxyborohydride and the mixture was further stirred at room temperature for 15 hours. The mixture was concentrated to dryness in vacuum and re-dissolved in 6 ml ethyl acetate, 10 ml of MTBE and 0.12 g of KOH dissolved in 5 ml of water. The organic phase washed with sodium hydrogenocarbonate until pH 5-6 and 3 times with 10 ml of water. The organic solution was dried on sodium sulfate and concentrated to dryness. Crude amine was further purified by column chromatography over silica gel (ethyl acetate 10:triethylamine 1; R_{f}=0.35) delivering 109 mg of a colorless amorphous foam (45%). LC-MS: M+1: 559. Retention time: 5.44 min (API).

¹HNMR (500 MHz, CDCl₃) δ: 7.45-7.25 (m, 4.5H), 7.25-7.0 (m, 3H), 6.9, 6.75 (2×bs, 0.6H), 3.85 (bm, 0.6H), 3.51 (bdd, J=12.5, 10.1 Hz, 1H), 3.5-2.75 (m, 9H), 2.7 (bs, 2H), 2.6 (bs, 4H), 2.4-1.7 (m, 9H), 1.65 (bs, 4H).).

¹³CNMR (125 MHz, CDCl₃) δ: 173.3, 171.5, 142.9, 136.4, 130.5, 130.2, 129.5, 128.4, 127.3, 126.8, 63.2, 56.1, 55.8, 53.3, 51.8, 51.3, 45.0, 42.5, 41.7, 38.8, 37.9, 30.6, 28.0, 24.1.

### EXAMPLE 5

### Synthesis of (2R)-1′-[(3S)-4-[benzoyl(methyl)amino]-3-(3,4-dichlorophenyl)-butyl]-N,N-dimethyl-1,4′-bipiperidine-2-carboxamide (example compound 100 in RS-config)

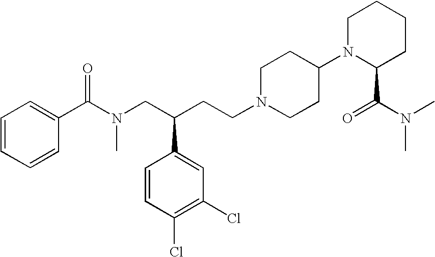

1 g of (7.75 mmole) L-pipecolic acid and 1.6 g (8.0 mmole) N-boc-piperidin-4-one were dissolved in 20 ml dichloromethane. To this solution 0.48 ml acetic acid was added and after 30 min., 3.27 g (2 eq) of sodium triacetoxyborohydride were added. The resulting suspension was stirred for 24 h at room temperature. The reaction mixture was diluted with 30 ml of a saturated solution of sodium chloride in water and the product was extracted with 6×20 ml dichloromethane. The organic phase was dried over Na₂SO₄ and evaporated under reduced pressure to yield 2.4 g of [1,4′]-bipiperidinyl-2,1′-dicarboxylic acid 1′-tert-butyl ester.

2.4 g (7.7 mmole) of [1,4′]-bipiperidinyl-2,1′-dicarboxylic acid 1′-tert-butyl ester were dissolved in 20 ml dichloromethane and 8 ml (2M/THF, 16 mmole) dimethylamine, 1.75 ml (16 mmole) N-methyl-morpholine, 2.1 g (16 mmole) N-hydroxymethylbenzotriazole were added. Finally 3 g (16 mmole) of EDC *HCl (ethyl-n′-(3-dimethylaminopropyl)-carbodiimide HCl salt) were added and reaction mixture was stirred for 20 h at room temperature. The reaction mixture was diluted with 50 ml ethyl acetate and washed with 2×20 ml 10% K₂CO₃/water solution. The organic phase was separated, dried over anhydrous sodium sulfate and evaporated under reduced pressure. The crude amide was purified by column chromatography over silica gel (ethyl acetate 2:ethanol 1, R_{f}=0.45) yielding 1.4 g colorless solid 2-dimethylcarbamoyl-[1,4′]-bipiperidinyl-1′-carboxylic acid tert-butyl ester (53%). [α]²²_{D}=+47°, c=1, MeOH.

1.4 g (4.13 mmole) 2-dimethylcarbamoyl-[1,4′]-bipiperidinyl-1′-carboxylic acid tert-butyl ester were dissolved in 1 ml dichloromethane and then 10 ml 6M HCl/isopropanol were added. The reaction mixture was stirred at room temperature for 3 h in which time, product crystallized in the reaction medium. The suspension was diluted with 50 ml MTBE, allowed to stir 30 more minutes, then filtered and washed with MTBE. The solid was dried under high vacuum for 1 h to remove traces of volatile materials yielding 1.17 g white powder [1,4′]-bipiperidinyl-2-carboxylic acid dimethylamide (91%).

To a suspension of 150 mg of the aldehyde from example 1 (0.0428 mmole), 135 mg (0.0428 mmole) of [1,4′]-bipiperidinyl-2-carboxylic acid dimethylamide and 0.5 g sodium acetate in 21 ml of THF at room temperature under stirring were added 0.05 ml of acetic acid and the mixture was stirred for 5 hours at room temperature. To the mixture were added portion wise 0.13 g (0.062 mmole) of sodium triacetoxyborohydride and the mixture was further stirred at room temperature for 15 hours. The mixture was concentrated to dryness in vacuum and re-dissolved in 6 ml ethyl acetate, 10 ml of MTBE and 0.12 g of KOH dissolved in 5 ml of water. The organic phase washed with sodium hydrogenocarbonate until pH 5-6 and 3 times with 10 ml of water. The organic solution was dried on sodium sulfate and concentrated to dryness. Crude amine was further purified by column chromatography over silica gel (ethyl acetate 10:methanol 1:triethyl amine 1; R_{f}=0.35) delivering 121 mg of a colorless amorphous foam (49%). LC-MS: M+1=573. Retention time: 4.73 min (API). [α]²²_{D}=−46.5°: c=1, CHCl₃.

¹HNMR (500 MHz, CDCl₃) δ: 7.45-7.3 (m, 4.5H), 7.25-7.0 (m, 2.8H), 6.9, 6.75 (2×bs, 0.65H), 3.85 (bm, 0.65H), 3.6-3.4 (m, 1H), 3.55 (dd, J=13.1, 9.5 Hz, 1H), 3.25 (bs, 3H), 3.0 (m, 2H), 2.9 (s, 3H), 2.75 (m, 2H), 2.6 (bs, 2H), 2.4 (m, 1H), 2.2 (dt, J=11.0, 2.4 Hz, 1H), 2.18 (m, 1H), 2.0-1.3 (m, 14H), 1.25 (m, 1H).

¹³CNMR (125 MHz, CDCl₃) δ: 173.1, 171.6, 142.9, 136.4, 132.7, 132.5, 130.5, 130.2, 129.5, 128.4, 127.2, 126.7, 63.9, 58.7, 55.9, 53.8, 53.6, 53.4, 45.5, 42.3, 41.4, 38.8, 36.8, 36.2, 34.5, 33.5, 30.8, 30.3, 28.9, 25.8, 24.3, 23.9.

### EXAMPLE 6

### Synthesis of (2S)-1′-[(3S)-4-[benzoyl(methyl)amino]-3-(3,4-dichlorophenyl)-butyl]-N,N-dimethyl-1,4′-bipiperidine-2-carboxamide (example compound 100 in SS-config.)

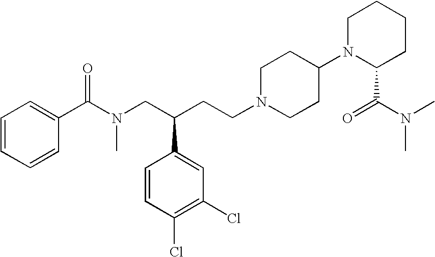

See the procedure for example 5. Instead of L-pipecolic acid, D-pipecolic acid is used yielding 1.267 g of white crystalline amine salt (2S)-1′-[(3S)-4-[benzoyl(methyl)amino]-3-(3,4-dichlorophenyl)butyl]-N,N-dimethyl-1,4′-bipiperidine-2-carboxamide and after the consequent steps including purification (ethyl acetate 10:methanol 1:triethyl amine 1; R_{f}=0.35) to 69 mg colorless amorphous foam (28%). LC-MS: M+1=573. Retention time: 4.73 min (API). [α]²²_{D}=−16.2°, c=1, CHCl₃.

¹HNMR (500 MHz, CDCl₃) δ: 7.45-7.3 (m, 4.5H), 7.25-7.0 (m, 2.8H), 6.9, 6.75 (2×bs, 0.65H), 3.85 (bm, 0.65H), 3.6-3.4 (m, 1H), 3.55 (m, 1H), 3.23 (bs, 3H), 3.0 (m, 2H), 2.95 (s, 3H), 2.68 (m, 2H), 2.4 (m, 1H), 2.2 (dt, J=11.0, 2.4 Hz, 1H), 2.18 (m, 1H), 2.0-1.3 (m, 14H), 1.25 (m, 1H).

¹³CNMR (125 MHz, CDCl₃) δ: 173.1, 171.7, 142.8, 136.0, 132.5, 130.6, 130.2, 129.5, 128.4, 127.2, 126.7, 63.8, 58.7, 56.0, 54.1, 53.4, 53.2, 45.5, 42.5, 41.5, 38.9, 36.8, 36.2, 34.5, 33.7, 30.7, 30.2, 28.9, 25.7, 24.2, 23.8.

### EXAMPLE 7

### Synthesis of 1′-[(3S)-4-[benzoyl(methyl)amino]-3-(3,4-dichlorophenyl)-butyl]-N,N-dimethyl-1,4′-bipiperidine-2-carboxamide (example compound 100)

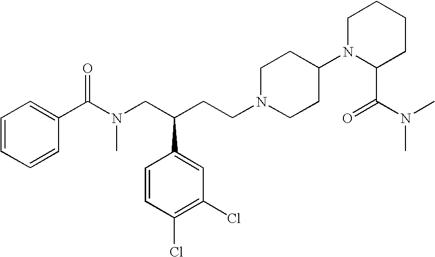

See the procedure for example 5. Instead of L-pipecolic acid or D-pipecolic acid, (D-L)-pipecolic acid is used yielding 1.267 g of white crystalline amine salt (2)-1′-[(3S)-4-[benzoyl(methyl)amino]-3-(3,4-dichlorophenyl)butyl]-N,N-dimethyl-1,4′-bi-piperidine-2-carboxamide and after the consequent steps including purification (ethyl acetate 10:methanol 1:triethyl amine 1; R_{f}=0.35) to 69 mg colorless amorphous foam (28%). LC-MS: M+1=573. Retention time: 4.73 min (API). [α]²²_{D}=−16.2°, c=1, CHCl₃.

¹HNMR (500 MHz, CDCl₃) δ: 7.45-7.3 (m, 4.5H), 7.25-7.0 (m, 2.8H), 6.9, 6.75 (2×bs, 0.65H), 3.85 (bm, 0.65H), 3.6-3.4 (m, 1H), 3.55 (m, 1H), 3.23 (bs, 3H), 3.0 (m, 2H), 2.95 (s, 3H), 2.68 (m, 2H), 2.4 (m, 1H), 2.2 (dt, J=11.0, 2.4 Hz, 1H), 2.18 (m, 1H), 2.0-1.3 (m, 14H), 1.25 (m, 1H).

¹³CNMR (125 MHz, CDCl₃) δ: 173.1, 171.7, 142.8, 136.0, 132.5, 130.6, 130.2, 129.5, 128.4, 127.2, 126.7, 63.8, 58.7, 56.0, 54.1, 53.4, 53.2, 45.5, 42.5, 41.5, 38.9, 36.8, 36.2, 34.5, 33.7, 30.7, 30.2, 28.9, 25.7, 24.2, 23.8.

### EXAMPLE 8

### Synthesis of N-[(2S)-2-(3,4-dichlorophenyl)-4-(4-{1-[(dimethylamino)-carbonyl]cyclohexyl}piperazin-1-yl)butyl]-N-methylbenzamide (example compound 96)

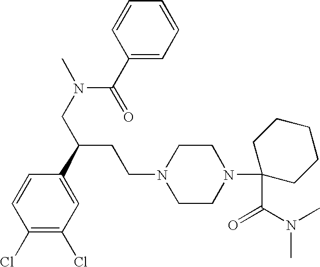

### Here: synthesis of 1-(4-benzylpiperazin-1-yl)-N,N-dimethylcyclo-hexanecarboxamide as starting material

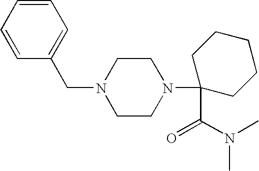

7.9 g (0.0262 mole) of 1-(4-benzylpiperazin-1-yl)-cyclohexanecarboxamide (known from WO0058292) in 120 ml of anhydrous THF was added drop wise at room temperature under stirring to a suspension of 2.83 g (0.071 mole) of sodium hydride 60% in oil in 120 ml of anhydrous THF under nitrogen. The mixture was then heated to 60° C. for 2 hours under stirring. After cooling down of the reaction mixture to room temperature, a solution of 6.33 g of methyl iodide in 80 ml of anhydrous DMF was added at room temperature and the reaction mixture was further stirred for 5 days. The reaction mixture was then poured on 500 g of iced water and the product extracted with 500 ml MTBE. The organic layer was recovered and washed with 500 ml of a saturated solution on sodium chloride in water. The organic phase was then dried on sodium sulfate and concentrated to dryness. The HPLC-MS shows the presence of the expected compound as well as of its monomethylated analog. The mixture was then separated by column chromatography on silicagel with n-hexane 1/ethyl acetate 9 to give 3.2 g of the expected compound (yield: 38%).

### Here: synthesis of N,N-dimethyl-1-piperazin-1-ylcyclohexane-carboxamide as starting material

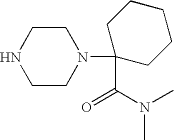

3.2 g (0.0097 mole) of the 1-(4-benzylpiperazin-1-yl)-N,N-dimethylcyclo-hexanecarboxamide was dissolved in 100 ml ethanol and 0.7 ml of acetyl chloride were added. Subsequently 1 g of 10% palladium on charcoal was added and the suspension was hydrogenated at 3 bars at room temperature for 15 hours. The catalyst was separated by filtration and washed with 25 ml ethanol. The solvent was then distilled off to deliver 2.6 g of N,N-dimethyl-1-piperazin-1-ylcyclohexane-carboxamide as a crystalline hydrochloride (yield: 97%). Melting point: 230-1° C.

### Synthesis of N-[(2S)-2-(3,4-dichlorophenyl)-4-(4-{1-[(dimethylamino)-carbonyl]cyclohexyl}piperazin-1-yl)butyl]-N-methylbenzamide (example compound 96)

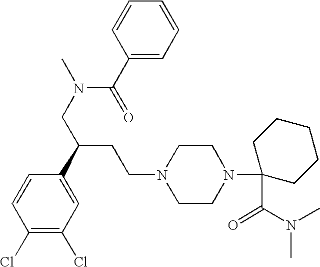

To a suspension of 150 mg of the aldehyde from example 1 (0.0428 mmole), 130 mg (0.0428 mmole) of N,N-dimethyl-1-piperazin-1-ylcyclohexane-carboxamide hydrochloride and 0.5 g sodium acetate in 21 ml of THF at room temperature under stirring were added 0.05 ml of acetic acid and the mixture was stirred for 5 hours at room temperature. To the mixture were added portion wise 0.13 g (0.062 mmole) of sodium triacetoxyborohydride and the mixture was further stirred at room temperature for 15 hours. The mixture was concentrated to dryness in vacuum and re-dissolved in 6 ml ethyl acetate, 10 ml of MTBE and 0.12 g of KOH dissolved in 5 ml of water. The organic phase washed with sodium hydrogenocarbonate until pH 5-6 and 3 times with 10 ml of water. The organic solution was dried on sodium sulfate and concentrated to dryness. Crude amine was further purified by column chromatography over silica gel (ethyl acetate 10:methanol 1:triethyl amine 1; R_{f}=0.35) delivering 260 mg of a colorless amorphous foam (85%) as dihydrochloride. LC-MS: M+1 (monoisotope): 585. Retention time: 5.22 min (API).

¹HNMR (as hydrochloride) (500 MHz, CD₃ OD) δ: 7.65-6.95 (m, 8H), 3.82 (m, 2H), 3.80-3.5 (m, 3H), 3.2-2.9 (m, 15H), 2.8 (m, 2H), 2.5-1.1(m, 12H).

¹³CNMR (125 MHz, CD₃ OD) δ: 174.1, 142.7, 137.3, 133.8, 132.5, 132.1, 129.7, 129.1, 128.1, 127.7, 58.2, 56.0, 53.7, 51.7, 47.3, 42.1, 39.2, 28.5, 27.1, 25.1

### EXAMPLE 9

### Synthesis of 1-[(3S)-4-[benzoyl(methyl)amino]-3-(3,4-dichlorophenyl)butyl]-4-cyclohexyl-N,N-dimethyl-piperidine-4-carboxamide (example compound 95)

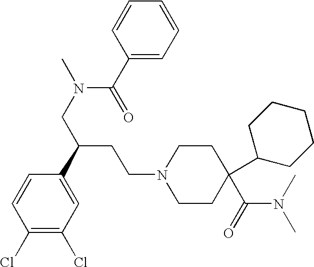

5 g (13.25 mmole) 4-phenyl-4-piperidinecarboxylic acid tosylate salt was dissolved in 50 ml of dichloromethane, then 3.75 ml (27 mmole) triethylamine and 3.1 g (14 mmole) bis-tert-butyloxycarbonate were added. The reaction mixture was stirred for 16 hours, then diluted with 100 ml of ethyl acetate and washed with 1×50 ml 10% aqueous acetic acid and with 3×50 ml of a saturated solution of sodium chloride in water. The organic phase was separated, dried over anhydrous sodium sulfate and evaporated under reduced pressure yielding 3.97 g white crystals of N-boc-4-phenyl-4-piperidinecarboxylic acid (98%).

13 mmol N-Boc-4-phenyl-4-piperidinecarboxylic acid was dissolved in 100 ml ethanol and hydrogenated with hydrogen (6 bars) over 5%-Rh/Al₂O₃ at 60° C. for 20 hours. After filtration and evaporation of solvent, pure crystalline N-Boc-4-cyclohexyl-4-piperidinecarboxylic acid was isolated 4.0 g (100%). Melting point: 156° C.

1 g (3.2 mmole) of N-boc-4-cyclohexyl-4-piperidine-carboxylic acid was dissolved in 20 ml of dichloromethane and one drop of dimethylformamide was added. To the resulting solution 0.29 ml (1.05 eq) oxalyl chloride was added and solution stirred for 30 min. 3.2 ml of dimethylamine (2M/THF, 2 eq) was added and mixture was stirred for 30 more minutes. Solution was diluted with 50 ml ethyl acetate and washed with 2×20 ml aqueous 10% potassium carbonate. The organic phase was dried over anhydrous sodium sulfate and evaporated under reduced pressure. Crude amide was purified by column chromatography over silica gel (dichloromethane 20:acetone 1, R_{f}=0.3) yielding to 217 mg of N-boc-4-cyclohexyl-4-piperidine-carboxylic acid dimethylamide as a colorless oil (20%).

217 mg (0.64 mmole) N-boc-4-cyclohexyl-4-piperidine-carboxylic acid dimethylamide was dissolved in 0.5 ml of dichloromethane and then 2 ml 6M HCl/isopropanol were added. The reaction mixture was stirred at room temperature for 3 hours in which time product crystallized in the reaction medium. The suspension was diluted with 20 ml MTBE, allowed to stir 30 more minutes and then filtered and washed with MTBE. The solid was dried under high vacuum for 1 hour to remove traces of volatile materials yielding 157 mg of 4-cyclohexyl-4-piperidinecarboxylic acid dimethylamide hydrochloride (90%) as a white powder.

To a suspension of 95 mg (0.027 mmole) of the aldehyde from example 1, 75 mg (0.0428 mmole) of 4-cyclohexyl-4-piperidinecarboxylic acid dimethylamide hydrochloride and 0.3 g sodium acetate in 20 ml of THF at room temperature under stirring were added 0.03 ml of acetic acid and the mixture was stirred for 5 hours at room temperature. To the mixture were added portion wise 0.1 g (0.045 mmole) of sodium triacetoxyborohydride and the mixture was further stirred at room temperature for 15 hours. The mixture was concentrated to dryness in vacuum and re-dissolved in 6 ml ethyl acetate, 10 ml of MTBE and 0.12 g of KOH dissolved in 5 ml of water. The organic phase washed with sodium hydrogenocarbonate until pH 5-6 and 3 times with 10 ml of water. The organic solution was dried on sodium sulfate and concentrated to dryness. Crude amine was further purified by column chromatography over silica gel (ethyl acetate 10:triethylamine 1; R_{f}=0.35) delivering 102 mg of a colorless amorphous foam (66%). LC-MS: M+1: 572. Retention time: 5.57 min (API).

¹HNMR (500 MHz, CDCl₃) δ: 7.45-7.3 (m, 4.5H), 7.25-7.0 (m, 2.7H), 6.9, 6.75 (2×bs, 0.65H), 3.8 (m, 0.5H), 3.6-3.4 (m, 1.5H), 3.15 (m, 0.6H), 3.0 (s, 6H), 2.9-2.5 (m, 4H), 2.2 (m, 0.6H), 2.1 (bd, J=12.5 Hz, 2H), 2.05-1.8 (m, 4H), 1.78 (bd, J=11.6 Hz, 2H), 1.6, (m, 6H), 1.3-1.0 (m, 5H).

¹³CNMR (125 MHz, CDCl₃) δ: 173.6, 170.5, 143.0, 135.4, 131.4, 129.6, 129.5, 129.2, 128.4, 127.3, 126.5, 126.2, 125.7, 55.4, 52.3, 51.1, 50.6, 49.5, 42.7, 41.5, 40.6, 37.7, 32.5, 29.7, 27.1, 26.2, 25.5.

### EXAMPLE 11

### Synthesis of N-{(2S)-2-(3,4-dichlorophenyl)-4-[4-pyrrolidin-1-yl-4-(pyrrolidin-1-ylcarbonyl)piperidin-1-yl]butyl}-N-methylbenzamide (example compound 103)

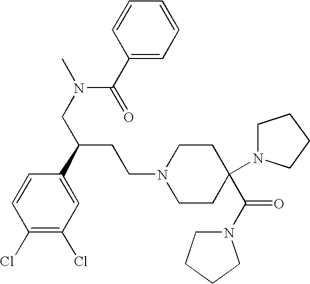

### Here: synthesis of 1-benzyl-4-pyrrolidin-1-yl-4-(pyrrolidin-1-ylcarbonyl)piperidine as starting material

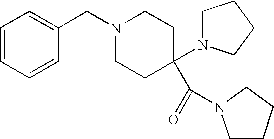

2.34 ml (0.0132 mole) of N-benzylpiperidin-4-one, 4.4 ml (0.0527 mole) of pyrrolidine, 1.6 ml (0.02 mole) of chloroform and 38 mg of n-benzyl triethylammonium chloride were stirred at 5° C. To the stirred mixture and by maintaining the temperature at 5° C., was added drop wise a solution of 2.6 g of sodium hydroxide in 5 ml of water. After 15 hours of stirring at 5° C., 50 ml of water were added and the organic material was extracted 3 times with 10 ml of methylene chloride. The organic phase was separated and washed twice with 30 ml of water. The organic phase was then separated, dried on sodium sulfate and concentrated to dryness delivering 3.426 g of an oily material showing the presence in NMR and MS of the expected compound.

Purification: 3 g of the mixture were separated by MPLC on 112 g of SiO₂ with n-hexane-ethyl ester 1:1 as eluant mixture giving 1.94 g of the expected compound as white solid. (according to Lai, J. Org. Chem. 1980, p. 3671).

### Here: synthesis of 4-pyrrolidin-1-yl-4-(pyrrolidin-1-ylcarbonyl)-piperidine as starting material

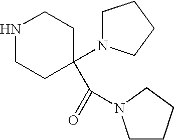

1.9 g (0.00556 mole) of 1-benzyl-4-pyrrolidin-1-yl-4-(pyrrolidin-1-ylcarbonyl)-piperidine was dissolved in 200 ml of ethanol at 30° C. and hydrogenated in the presence of 4 spatules of palladium hydroxide 20% on charcoal (moisture 60%) at 4 bars for 6 hours at 40° C. The catalyst was recovered by filtration and the solution concentrated to dryness delivering the expected de-benzylated compound which was identified in NMR and MS and used without further purification.

### Synthesis of N-{(2S)-2-(3,4-dichlorophenyl)-4-[4-pyrrolidin-1-yl-4-(pyrrolidin-1-ylcarbonyl)piperidin-1-yl]butyl}-N-methylbenzamide (example compound 103)

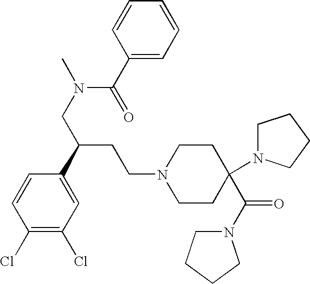

To a suspension of 180 mg (0.51 mmole) of the aldehyde from example 1, 180 mg (0.51 mmole) of 4-pyrrolidin-1-yl-4-(pyrrolidin-1-ylcarbonyl)-piperidine and 0.3 g sodium acetate in 20 ml of THF at room temperature under stirring were added 0.03 ml of acetic acid and the mixture was stirred for 5 hours at room temperature. To the mixture were added portion wise 0.2 g (0.09 mmole) of sodium triacetoxyboro-hydride and the mixture was further stirred at room temperature for 15 hours. The mixture was concentrated to dryness in vacuum and re-dissolved in 6 ml ethyl acetate, 10 ml of MTBE and 0.2 g of KOH dissolved in 5 ml of water. The organic phase washed with sodium hydrogen carbonate until pH 5-6 and 3 times with 10 ml of water. The organic solution was dried on sodium sulfate and concentrated to dryness. Crude amine was further purified by column chromatography over silica gel (ethyl acetate 10:triethylamine 1; R_{f}=0.35) giving 236 mg of a colorless amorphous foam (79%). LC-MS: M+1 (monoisotope): 585. Retention time: 5.85 min (API).

¹HNMR (as base) (500 MHz, CDCl₃) δ: 7.45-6.70 (m, 8H), 3.85 (m, 2H), 3.6-3.4 (m, 4H), 3.3-2.5 (m, 10H), 2.35-1.9 (m, 8H), 1.78 (s, 4H), 1.67 (s, 4H).

¹³CNMR (125 MHz, CDCl₃) δ: 172.4, 171.6, 143.0, 136.5, 132.4, 130.5, 129.4, 128.3, 127.3, 126.7, 62.9, 56.1, 53.3, 51.7, 51.1, 47.5, 45.1, 41.7, 38.7, 27.9, 27.4, 24.0, 23.2

### EXAMPLE 12

### Synthesis of 3-cyano-N-{(2S)-2-(3,4-dichlorophenyl)-4-[4-pyrrolidin-1-yl-4-(pyrrolidin-1-ylcarbonyl)piperidin-1-yl]butyl}-N-methyl-1-naphthamide (example compd 102)

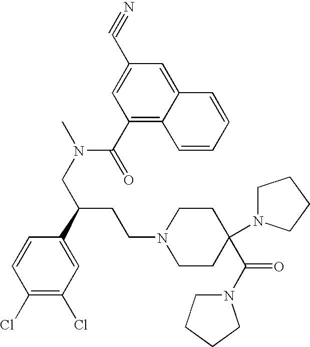

### Here: synthesis of 3-Cyano-naphthalene-1-carboxylic acid [2S-(3,4-dichlorophenyl)-4-oxo-butyl]-methyl-amide as starting material

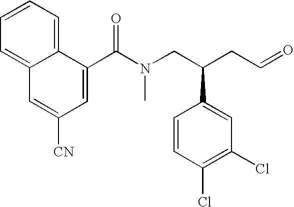

12.2 g DMSO in 100 ml dichloromethane are added drop wise to 7.3 g oxalyl chloride in 100 ml dichloromethane unter nitrogen at −70° C. unter stirring. The resulting mixture was stirred for another 15 minutes before 20 g of [2S-(3,4-dichlorophenyl)-4-hydroxy-butyl]-methyl-carbamic acid tert-butyl ester in 200 ml dichloromethane were added. The mixture was stirred at −70° C. for one hour before 40.3 ml of triethylamine in 50 ml dichloromethane were added drop wise. The solution was stirred at −70° C. for 15 minutes and then allowed to warm up to room temperature. The solvent was removed and the residue was dissolved in 300 ml of toluene and 200 ml of ethyl acetate. The resulting solution washed six times with 200 ml of a saturated solution of NaCl in water, dried over sodium sulfate and concentrated to dryness to deliver 19.7 g of 3-Cyano-naphthalene-1-carboxylic acid [2S-(3,4-dichlorophenyl)-4-oxo-butyl]-methyl-amide.

### Synthesis of 3-cyano-N-{(2S)-2-(3,4-dichlorophenyl)-4-[4-pyrrolidin-1-yl-4-(pyrrolidin-1-ylcarbonyl)piperidin-1-yl]butyl}-N-methyl-1-naphthamide (example compound 102)

253.8 mg (0.0006 mole) of 3-cyano-naphthalene-1-carboxylic acid [2S-(3,4-dichlorophenyl)-4-oxo-butyl]-methyl-amide, 150 mg (0.006 mole) of 4-pyrrolidin-1-yl-4-(pyrrolidin-1-ylcarbonyl)-piperidine and 72 mg (0.0012 mole) of acetic acid were dissolved in 20 ml of methylene chloride and stirred for 30 minutes at room temperature. Subsequently, 163.7 mg of sodium triacetoxyborohydride was added and the mixture stirred at room temperature for 3 hours. The reaction mixture was then diluted with 40 ml of ethyl acetate and the resulting phase washed twice with 50 ml a 10% aqueous solution of potassium carbonate and once with 50 ml of a saturated sodium chloride water solution. The organic phase was then separated and concentrated to dryness to deliver 280 mg of the title base as an amorphous solid. LC-MS: M+1 (monoisotope): 573. Retention time: 7.90 min (API).

¹HNMR (500 MHz, CDCl₃) δ: 8.24 (0.35H), 8.19(0.65H), 7.97-6.50) (m, 8H), 4.4 (bs, 0.65H), 3.85 (bs, 2H), 3.7-3.25 (m, 3.35H), 3.2 (s,m, 1H), 3.0-1.9 (m, 10H), 1.8 (s, 4H), 1.68 (s, 4H).

¹³CNMR (125 MHz, CDCl₃) δ: 172.4, 172.3, 142.5, 136.5, 134.7, 132.4, 131.0, 130.6, 130.5, 130.1, 128.3, 127.5, 126.7, 125.2, 123.9, 118.4, 109.0, 63.0, 57.1, 56.0, 53.3, 51.7, 51.1, 47.5, 45.1, 45.0, 41.7, 38.7, 37.7, 33.6, 31.6, 27.9, 27.4, 24.0

### EXAMPLE 13

### Synthesis of N-{(2S)-2-(3,4-dichlorophenyl)-4-[4′-(piperidin-1-ylcarbonyl)-1,4′-bipiperidin-1′-yl]butyl}-N-methylbenzamide (example compound 104)

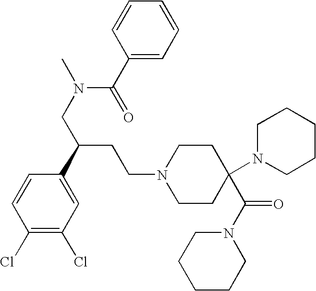

### Here: synthesis of 1′-benzyl-4′-(piperidin-1-ylcarbonyl)-1,4′-bipiperidine as starting material

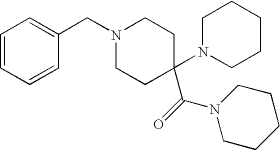

1′-benzyl-4′-(piperidin-1-ylcarbonyl)-1,4′-bipiperidine was obtained by condensation of 2.34 ml (0.00132 mole) of N-benzylpiperidin-4-one, 5.3 ml (0.00536 mole) of piperidine, 1.6 ml (0.002 mole) of chloroform and 38 mg of benzyltriethylammonium chloride. A raw mixture of 4.445 g of an oily material was obtained (yield: 90%), 2 g of which were subsequently purified in preparative HPLC with a gradient acetonitrile/water containing formic acid in 2 runs to give 678 mg of the product as diformiate after lyophilisation of the fractions containing the pure expected compound.

### Here: synthesis of 4′-(piperidin-1-ylcarbonyl)-1,4′-bipiperidine as starting material

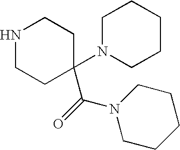

576 mg of 1′-benzyl-4′-(piperidin-1-ylcarbonyl)-1,4′-bipiperidine as diformiate was transformed into dihydrochloride, dissolved in 50 ml ethanol and hydrogenated at 40° C. for 4 days in presence of 50 mg of 10% palladium on charcoal. The catalyst was separated by filtration and washed 3 times with 50 ml ethanol. The organic phases were reunited and concentrated to dryness to deliver 4′-(piperidin-1-ylcarbonyl)-1,4′-bipiperidine as a solid dihydrochloride.

### Synthesis of N-{(2S)-2-(3,4-dichlorophenyl)-4-[4′-(piperidin-1-ylcarbonyl)-1,4′-bipiperidin-1′-yl]butyl}-N-methylbenzamide (example compound 104)

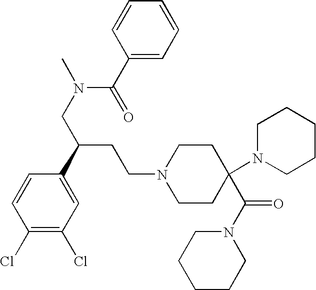

127 mg (0.00036 mole) of the aldehyde from example 1, 160 mg (0.0036 mole) 4′-(piperidin-1-ylcarbonyl)-1,4′-bipiperidine as a solid dihydrochloride and 35 mg (0.0006 mole) of acetic acid were dissolved in 20 ml of methylene chloride and stirred for 30 minutes at room temperature. Subsequently, 85 mg of sodium triacetoxyborohydride was added and the mixture stirred at room temperature for 3 hours. The reaction mixture was then diluted with 20 ml of ethyl acetate and the resulting phase washed twice with 50 ml a 10% aqueous solution of potassium carbonate and once with 50 ml of a saturated sodium chloride water solution. The organic phase was then separated and concentrated to dryness to deliver 206 mg of the title base as a colorless foam in form of the hydrochloride (quantitative yield). LC-MS: M+1 (monoisotope): 613. Retention time: 6.14 min (API).

¹HNMR (as hydrochloride)(500 MHz, CDCl₃ OD) δ: 7.65-6.95 (m, 8H), 3.8-3.55 (m, 8H), 3.35-3.25 (2×s, 3H), 3.2-2.4 (m, 13H), 2.35-2.15 (m, 2H), 1.95 (s, 4H) 1.7 (s, 2H), 1.62 (s, 4H).

¹³CNMR (125 MHz, CD₃ OD) δ: 174.1, 142.7, 137.3, 133.8, 132.5, 132.1, 129.7, 129.1, 128.1, 127.7, 58.2, 56.0, 53.7, 51.7, 47.3, 42.1, 39.2, 28.5, 27.1, 25.1

### EXAMPLE 13

### Synthesis of N-{(2S)-2-(3,4-dichlorophenyl)-4-[4′-(pyrrolidin-1-ylcarbonyl)-1, 4′-bipiperidin-1′-yl]butyl}-N-methylbenzamide (example compound 124)

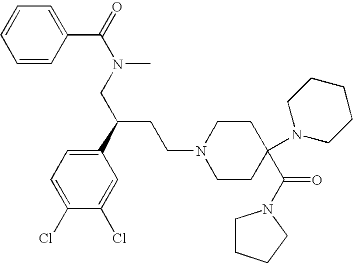

### Here: synthesis of 1′-benzyl-1,4′-bipiperidine-4′-carbox-amide as starting material

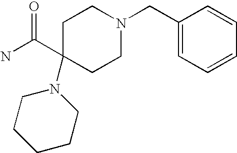

4.5 g (52.8 mmole) of piperidine, 5 g (26.4 mmole) N-benzyl-piperi-din-4-one, 9.5 g (3 eq) of magnesium sulfate and 2.3 ml of N-dimethylacetamide were mixed together and then 2.6 ml (1 eq) of 2-cyano-2-hydroxy-propane were added. The resulting suspension was stirred over 48 h at 55° C. whereupon pasty suspension solidifies. Crude product was mixed with 100 ml water and 100 ml ethyl acetate. Organic phase washed with water (2×50 ml), dried over sodium sulfate and evaporated yielding 7 g crude aminonitrile.

1.3 g (3.6 mmole) of the resulting aminonitrile was dissolved in 15 ml 90% wt. sulfuric acid and heated for 10 minutes at 100° C. The resulting solution was poured on ice and then basicified to pH 9 with sodium hydroxide. Crude amine was extracted with ethyl acetate (3×30 ml), organic phase washed once with a saturated solution of sodium chloride in water, dried over sodium sulfate and evaporated. Yield 1 g (92%) light yellow crystals of 1′-benzyl-1,4′-bipiperidine-4′-carboxamide.

### Here: synthesis of N,N-diallyl-1′-benzyl-1,4′-bipiperidine-4′-carboxamide as starting material

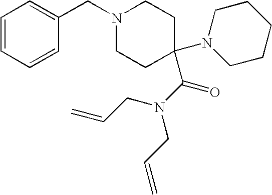

1.75 g (5.83 mmole) of 1′-benzyl-1,4′-bipiperidine-4′-carboxamide was dissolved in 10 ml hexamethylphosphortriamide. To this solution 466 mg (2 eq) sodium hydride (60% in oil) was added portion-wise and the suspension was stirred at 60° C. for 2 hours. Then the resulting solution was cooled to room temperature and 1 ml (2 eq) allyl bromide was added over 6 hours via syringe pump. After 24 h reaction was quenched by addition of 10% aqueous ammonium chloride (50 ml) and product extracted with ethyl acetate. The organic phase dried over sodium sulfate and evaporated. Crude compound was purified by column chromatography over silica gel (ethyl acetate 5:ethanol 1, R_{f}=0.3) yielding 1 g (45%) of desired N,N-diallyl-1′-benzyl-1,4′-bipiperidine-4′-carboxamide.

### Here: synthesis of 1′-benzyl-4′-(2,5-dihydro-1H-pyrrol-1-ylcarbonyl)-1,4′-bipiperidine as starting material

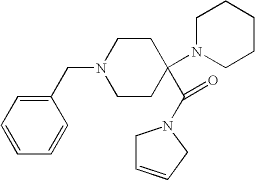

1 g (2.6 mmole) N,N-diallyl-1′-benzyl-1,4′-bipiperi-dine-4′-carboxamide was dissolved in 50 ml dichloromethane and to the resulted refluxed solution was added portion wise Grubbs-1 catalyst (1 g, 50 mol %) over 36 hours. The solvent was evaporated and product was purified by column chromatography over silica gel (ethyl acetate, R_{f}=0.35) yielding 640 mg (70%) of pure 1′-benzyl-4′-(2,5-dihydro-1H-pyrrol-1-ylcarbonyl)-1,4′-bipiperidine.

### Here: synthesis of 4′-(pyrrolidin-1-ylcarbonyl)-1,4′-bipiperidine as starting material

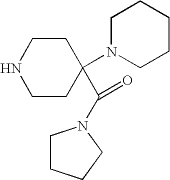

0.64 g (1.81 mmole) of 1′-benzyl-4′-(2,5-dihydro-1H-pyrrol-1-ylcarbonyl)-1,4′-bipiperidine were dissolved in 70 ml ethanol and 200 mg of Pd(OH) 20% over carbon (60% moisture) were added. The compound was hydrogenated with hydrogen under 5 bar pressure. After 4 hours, the catalyst was removed by filtration and solvent evaporated under reduced pressure yielding 470 mg (99%) of blue-gray crystalline powder. Melting point: 144° C.

### Synthesis of N-{(2S)-2-(3,4-dichlorophenyl)-4-[4′-(pyrrolidin-1-ylcarbonyl)-1,4′-bipiperi-din-1′-yl]butyl}-N-methylbenzamide (example compound 124)

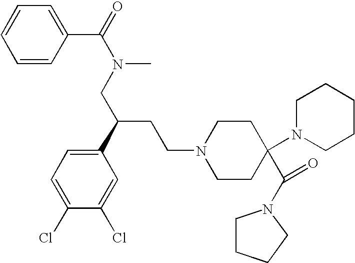

150 mg (0.005 mole) of the aldehyde from example 1, 120 mg (0.005 mole) 4′-(pyrrolidin-1-ylcarbonyl)-1,4′-bipiperidine and 35 mg (0.0006 mole) of acetic acid were dissolved in 20 ml of methylene chloride and stirred for 30 minutes at room temperature. Subsequently, 85 mg of sodium triacetoxyborohydride was added and the mixture stirred at room temperature for 3 hours. The reaction mixture was then diluted with 20 ml of ethyl acetate and the resulting phase washed twice with 50 ml a 10% aqueous solution of potassium carbonate and once with 50 ml of a saturated sodium chloride water solution. The organic phase was then separated and concentrated to dryness to deliver the title compound. After purification, 171 mg of N-{(2S)-2-(3,4-dichlorophenyl)-4-[4′-(pyrrolidin-1-ylcarbonyl)-1,4′-bipiperidin-1′-yl]butyl}-N-methylbenzamide (66%) were isolated. C-MS: M+1=598. Retention time: 5.82 min (API).

¹HNMR (500 MHz, CDCl₃) δ: 7.45-7.3 (m, 4.6H), 7.25-7.0 (m, 2.8H), 6.9, 6.75 (2×bs, 0.65H), 4.1-3.7 (m, 2H), 3.6-3.4 (m, 3.65H), 3.15 (bs, 0.65H), 3.0 (bs, 1H), 2.9-2.6 (m, 5H), 2.46 (s, 4H), 2.3-1.85 (m, 8H), 1.75 (bs, 7H), 1.5 (m, 4H), 1.4 (m, 2H).

¹³CNMR (125 MHz, CDCl₃) δ: 172.4, 171.6, 142.9, 136.4, 132.4, 130.5, 130.2, 129.4, 128.3, 127.2, 126.7, 66.4, 57.1, 56.1, 55.7, 53.3, 51.6, 51.1, 47.8, 47.2, 42.5, 41.6, 38.7, 33.6, 30.9, 27.4, 27.1, 26.9, 25.1, 23.2.

### EXAMPLE 14

### Alternative synthesis of 1′-[(3S)-4-[benzoyl(methyl)amino]-3-(3,4-dichlorophenyl)butyl]-N-ethyl-N-methyl-1,4′-bipiperidine-4′-carboxamide (example compound 95)

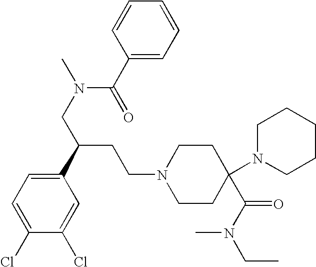

100 mg (0.158 mmole) of amide 1′-[4-(benzoyl-methyl-amino)-3-(3,4-dichlorophenyl)-butyl]-[1,4′]bipiperidinyl-4′-carboxylic acid methylamide were dissolved in 1 ml absolute DMSO and then 21 mg (0.19 mmole) of potassium tert-butylate were added under inert atmosphere. The resulting solution was stirred at room temperature for 1 h, then 25 mg (0.16 mmole) of ethyl iodide were added and the reaction mixture stirred for additional 24 h at room temperature. The reaction mixture was diluted with 10 ml of 10% NH₄Cl water solution and product extracted with ethyl acetate (3×10 ml). The organic phase was dried over Na₂SO₄ and evaporated under reduced pressure. Crude product was purified by column chromatography over silica gel (ethyl acetate 5:methanol 1, R_{f}=0.25) yielding colorless foam (31 mg, 30%). LC-MS: M+1=587. Retention time: 5.59 min (API).

¹HNMR (500 MHz, CDCl₃) δ: 7.3 (m, 4.5H), 7.25-7.0 (m, 3H), 6.9, 6.7 (2×bs, 0.6H), 3.9 (bs, 0.6H), 3.5 (dd, J=12.97, 9.92 Hz, 1H), 3.4 (m, 3.5H), 3.2 (bs, 1H), 3.0-2.7 (m, 3H), 2.7 (s, 3H), 2.5 (bs, 4H), 2.4-1.6 (m, 10H), 1.5 (s, 4H), 1,4 (s, 2H), 1.1 (bs, 3H).

¹³CNMR (125 MHz, CDCl₃) δ: 173.0, 172.0, 136.3, 130.6, 130.2, 129.5, 128.4, 127.2, 126.8, 66.4, 56.2, 55.9, 53.4, 51.7, 51.1, 50.6, 47.3, 44.6, 44.1, 42.5, 41.6, 41.1, 38.8, 36.0, 34.0, 33.5, 30.2, 29.7, 29.4, 26.9, 25.1, 11.7.

### EXAMPLE 14

### Synthesis of 4-({[(2S)-2-(3,4-dichlorophenyl)-4-{4′-[(dimethylamino)-carbonyl]-1,4′-bipiperidin-1′-yl}butyl](methyl)amino}carbonyl)phenyl acetate (example compound 5)

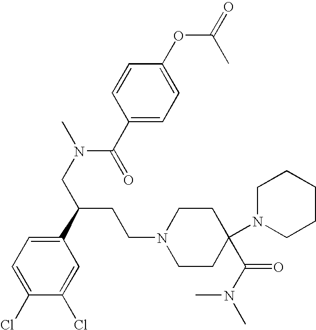

### Here: synthesis of tert-butyl[(2S)-2-(3,4-dichlorophenyl)-4-{4′-[(dimethylamino)-carbonyl]-1,4′-bipiperidin-1′-yl}butyl]methyl carbamate as starting material

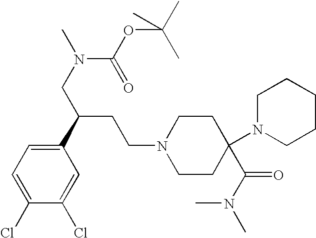

To a suspension of 4 g (0.0115 mole) [2-(3,4-dichlorophenyl)-4-oxo-butyl]-methyl-carbamic acid tert-butyl ester, 4.35 g (0.0139 mole) of the piperidinopiperidineketamide hydrochloride[1,4′]bipiperidinyl-4′-carboxylic acid dimethylamide and 2 g of sodium acetate in 200 ml of THF, 1.5 ml of acetic acid were added. The reaction mixture was stirred for 4 hours at room temperature. To the reaction mixture were the added portion-wise 4.88 g (0.0231 mole) of sodium triacetoxyborohydride and the solution was stirred for 15 hours at room temperature and subsequently concentrated to dryness. The remaining material was dissolved in 100 ml of MTBE and 5 g of potassium hydroxide in 50 ml of water. The separated organic layer was then washed three times with 50 ml water, dried on sodium sulfate and concentrated to deliver 6.06 g of a foam which was used in the next step without further purification. Yield: 92%

### Here: synthesis of 1′-[(3S)-3-(3,4-dichlorophenyl)-4-(methylamino)butyl]-N,N-dimethyl-1,4′-bipiperidine-4′-carboxamide as starting material

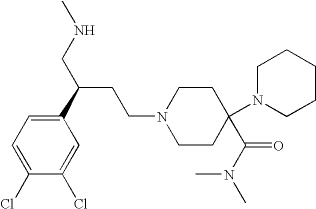

6 g (0.0105 mole) of [2-(3,4-dichlorophenyl)-4-(4′-dimethylcarbamoyl-[1,4′]-bipiperidinyl-1′-yl)-butyl]-methyl-carbamic acid tert-butyl ester were dissolved in 10 ml methylene chloride and 40 ml of 5N solution of HCl in isopropanol (0.2 mole) at room temperature. A precipitate of 1′-[(3S)-3-(3,4-dichlorophenyl)-4-(methylamino)butyl]-N,N-dimethyl-1,4′-bipiperidine-4′-carboxamide as hydrochloride precipitated and the mixture was stirred for 15 hours till completeness of the reaction. The mixture was dropped in 150 ml MTBE and further stirred for 1 hour at room temperature. The precipitate was separated by filtration, washed 3 times with 10 ml of MTBE and the obtained solid dried under vacuum at 60° C. to deliver 4.1 g of 1′-[(3S)-3-(3,4-dichlorophenyl)-4-(methylamino)butyl]-N,N-dimethyl-1,4′-bipiperidine-4′-carboxamide as the trihydrochloride. Yield: 67%. Optical rotation: −2.0° (c=1% in methanol)

### Here: synthesis of 4-({[(2S)-2-(3,4-dichlorophenyl)-4-{4′-[(-N,N-dimethylamino)-carbonyl]1,4′-bipiperidin-1′-yl}butyl](methyl)amino}carbonyl)phenyl acetate

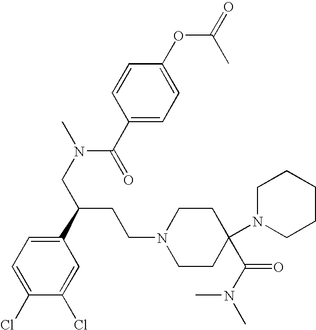

220 mg (0.00038 mole) of 1′-[(3S)-3-(3,4-dichlorophenyl)-4-(methylamino)butyl]-N,N-dimethyl-1,4′-bipiperidine-4′-carboxamide were dissolved in 20 ml of methylene chloride in the presence of 200 μl of triethylamine under stirring at room temperature. To the reaction mixture, were added drop wise at room temperature, a solution of 98 mg of 4-acetoxybenzoyl chloride in 20 ml of methylene chloride and subsequently 200 μl of triethylamine were added. The reaction mixture was stirred for 15 hours at room temperature and concentrated in vacuum. The residue was dissolved in 50 ml of ethyl acetate and 30 ml of MTBE in the presence of 200 mg potassium hydroxide dissolved in 20 ml of water. The organic phase was separated and washed 4 times with 20 ml of water. The organic layer was recovered, dried on sodium sulfate and concentrated to dryness to deliver 240 mg (quantitative yield) of 4-({[(2S)-2-(3,4-dichlorophenyl)-4-{4′-[(dimethylamino)carbonyl]-1,4′-bipiperidin-1′-yl}butyl](methyl)amino}carbonyl)phenyl acetate as a glassy material. LC-MS: M+1 (monoisotope): 631. Retention time: 7.78 min (API).

¹HNMR (as base) (500 MHz, CDCl₃) δ: 7.45-7.1 (m, 7H), 7.0, 6.8 (2×bs, 0.7H), 3.82 (bs, 0.7H), 3.55 (dd, 1H), 3.5-3.2 (m, 4H), 3.1 (bs, 0.7H), 3.0 (m, 2H), 2.95-2.90 (2×s, 3H), 2.8-2.5 (m, 4.5H), 2.3 (s, 3H) 2.25-1.6 (m, 12H), 1.5 (s, 4H), 1,4 (s, 2H).

¹³CNMR (125 MHz, CDCl₃) δ: 170.7, 168.2, 150.7, 131.7, 121.5, 120.9, 66.1, 55.2, 56.1, 52.6, 51.7, 50.9, 50.4, 46.6, 40.9, 38.0, 36.9, 29.9, 26.2, 24.4, 20.4

### EXAMPLE 15

### Synthesis of 1′-{(3S)-3-(3,4-dichlorophenyl)-4-[(4-hydroxybenzoyl)-(methyl)amino]butyl}-N,N-dimethyl-1,4′-bipiperidine-4′-carboxamide (example compound 6)

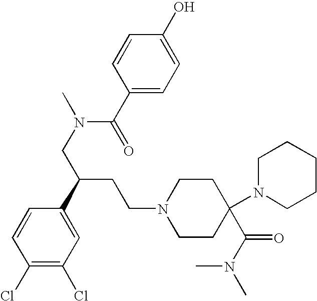

170 mg (0.00027 mole) of 4-({[(2S)-2-(3,4-dichlorophenyl)-4-{4′-[(dimethylamino)-carbonyl]-1,4′-bipiperidin-1′-yl}butyl](methyl)amino}carbonyl)phenyl acetate was dis-solved in 30 ml of methanol in the presence of 300 mg of potassium hydroxide at room temperature and stirred for 20 hours. The solution was then concentrated to dryness and dissolved in 50 ml of ethyl acetate, 40 ml of MTBE and a solution of 2 g of ammonium chloride in 20 ml of water until a pH7 was reached. The organic phase was separated, washed 3 times with 20 ml of water, dried on sodium sulfate, concentrated under vacuum to deliver 102 mg of 1′-{(3S)-3-(3,4-dichlorophenyl)-4-[(4-hydroxybenzoyl)(methyl)amino]-butyl}-N,N-dimethyl-1,4′-bipiperidine-4′-carboxamide as a glassy compound (yield: 64%). LC-MS: M+1 (monoisotope): 589. Retention time: 7.06 min (API).

¹HNMR (as base) (500 MHz, CDCl₃) δ: 7.40-6.70 (m, 7H), (2×bs, 0.7H), 3.88 (bs, 0.7H), 3.45 (dd, 1H), 3.5-3.25 (m, 4. H), 3.2 (bs, 0.7H), 3.0 (m, 2H), 2.95-2.90 (2×s, 3H), 2.8-2.5 (m, 4.5H), 2.4-1.6 (m, 12H), 1.5 (s, 4H), 1,4 (s, 2H).

¹³CNMR (125 MHz, CDCl₃) δ: 173.5, 172.8, 172.4, 160.1, 158.8, 132.9, 131.56, 131.4, 130.9, 129.8, 127.1, 70.6, 65.9, 55.6, 53.4, 51.7, 47.3, 38.7, 37.8, 31.9, 26.7, 26.0, 24.9

### EXAMPLE 16

### Synthesis of 1′-[(3S)-4-[benzoyl(methyl)amino]-3-phenylbutyl]-N,N-dimethyl-1, 4′-bipiperidine-4′-carboxamide

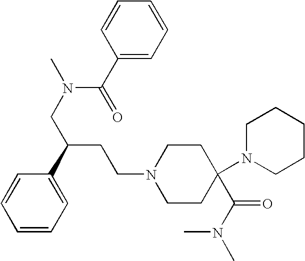

700 mg (0.00108 mole) of 1′-[4-(benzoyl-methyl-amino)-3-(3,4-dichlorophenyl)-butyl]-[1,4′]bipiperidinyl-4′-carboxylic acid methylamide dihydrochloride were dissolved in 100 ml ethanol at room temperature. To the solution were added 50 ml of water and 560 mg of potassium hydroxide. The solution was hydrogenated for 5 days at room temperature in the presence of 5 spatulas of 10% palladium on charcoal at 4 bars. The solution was recovered after filtration and separation of the catalyst, concentrated to dryness and subsequently dissolved in 60 ml MTBE. The organic phase washed 3 times with 10 ml of water, separated, dried on sodium sulfate and concentrated to dryness to give the title product as 405 mg of an oily material (Yield: 74%). 390 mg of the base were dissolved in 2 ml of ethanol at 40° C. and 330 μl of HCl 5N in IPA were added under stirring to give a solution to whom 20 ml of MTBE were added to give a precipitate. After heating of the solution to 50° C., the suspension was cooled to room temperature. The precipitate was recovered by filtration, washed twice with 10 ml of methanol, dissolved in methanol the concentration to dryness of which delivered 435 mg (98% yield) of a foamy colorless dihydrochloride. LC-MS: M+1 (monoisotope): 505. Retention Time: 4.94 min (API).

¹HNMR (as base) (500 MHz, CDCl₃) δ: 7.40-6.85 (m, 10H), 3.87 (bs, 0.7H), 3.58 (dd, 0.7H), 3.5-3.25 (m, 4.4H), 3.1 (bs, 0.7H), 2.96 (m, 2H), 2.95-2.85 (bs, 3H), 2.85-2.45 (m, 4.5H), 2.3-1.6 (m, 12H), 1.51 (s, 4H), 1,41 (s, 2H).

¹³CNMR (125 MHz, CDCl₃) δ: 173.5, 171.5, 142.4, 136.8, 129.2, 128.7, 128.5, 128.2, 128.0, 126.7, 66.9, 57.6, 56.4, 53.4, 51.8, 47.3, 42.4, 38.5, 37.7, 33.6, 31.1, 26.9, 25.1

### EXAMPLES 17 TO 36

Compounds 105 to 123 were obtained by reductive amination as outlined in process (a). An aldehyde is reductively aminated with an amine to yield compound of Formula I, examples 105 to 123

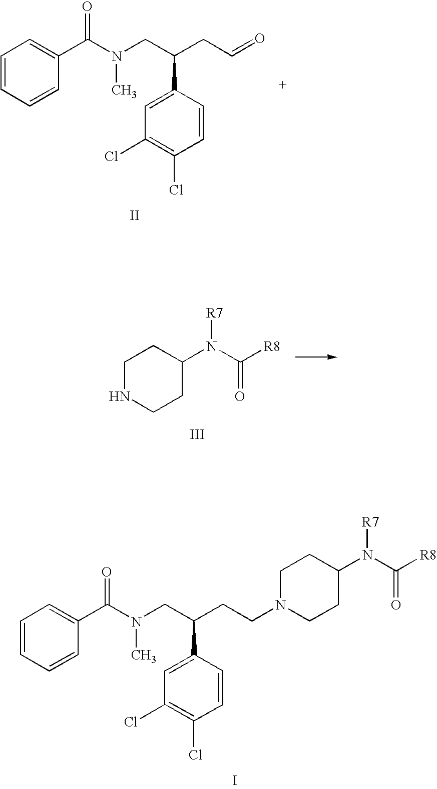

### Synthesis of N-[(2S)-4-{4-[(cyclopropylmethyl)(Propionyl)-amino]piperidin-1-yl}-2-(3,4-dichlorophenyl)butyl]-N-methylbenzamide (example compound 105)

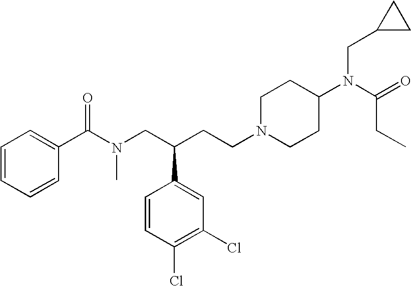

To a mixture of 105 mg (0.3 mmole) of N-[2-(3,4-Dichlorophenyl)-4-oxo-butyl]-N-methyl-benzamide (compound II), 70 mg (0.33 mmole) of N-(cyclopropylmethyl)-N-piperidin-4-ylpropanamide (compound II), and 37 mg (0.45 mmol) sodium acetate in 6 ml THF at room temperature under stirring were added in 0.03 ml (0.51 mmole) acetic acid. After 0.5 h stirring, 127 mg (0.6 mmole) of sodium triacetoxyborohydride were added to this mixture. After stirring for further 15 h, the mixture was concentrated under vacuum to dryness and re-dissolved in 20 ml of dichloromethane. The organic solution washed three times with potassium hydrogencarbonate and then dried on sodium sulfate. After filtration the organic phase was concentrated under vacuum to dryness. The crude product was purified by column chromatography on silica gel using ethyl acetate/ethanol as eluents (100/0 to 80/20 v/v) yielding 93 mg of a colorless oil.

The following compounds were prepared according to the described process of reductive amination:
N-[(2S)-2-(3,4-dichlorophenyl)-4-{4-[isopropyl(propionyl)amino]piperidin-1-yl}butyl]-N-methylbenzamide (compound example 106);
N-[(2S)-2-(3,4-dichlorophenyl)-4-{4-[phenyl(propionyl)amino]piperidin-1-yl}butyl]-N-methylbenzamide (compound example 107);
N-[(2S)-4-{4-[butyl(propionyl)amino]piperidin-1-yl}-2-(3,4-dichlorophenyl)butyl]-N-methylbenzamide (compound example 108);
N-[(2S)-4-{4-[butyl-(cyclopropylcarbonyl)amino]piperidin-1-yl}-2-(3,4-dichlorophenyl)butyl]-N-methyl-benzamide (compound example 109);
N-[(2S)-4-{4-[butyl(cyclohexylcarbonyl)-amino]piperidin-1-yl}-2-(3,4-dichlorophenyl)butyl]-N-methylbenzamide (compound example 110);
N-[(2S)-4-{4-[benzoyl(butyl)amino]piperidin-1-yl}-2-(3,4-dichlorophenyl)butyl]-N-methylbenzamide (compound example 111);
N-[(2S)-2-(3,4-dichlorophenyl)-4-{4-[(4-methoxybutyl)(propionyl)amino]piperidin-1-yl}butyl]-N-methylbenzamide (compound example 112);
N-[(2S)-4-{4-[(cyclopropylcarbonyl)(4-methoxybutyl)amino]piperidin-1-yl}-2-(3,4-dichlorophenyl)butyl]-N-methylbenzamide (compound example 113);
N-[(2S)-4-{4-[(cyclohexylcarbonyl)(4-methoxybutyl)-amino]piperidin-1-yl}-2-(3,4-dichlorophenyl)butyl]-N-methylbenzamide (compound example 114);
N-[(2S)-4-{4-[benzoyl(4-methoxybutyl)amino]piperidin-1-yl}-2-(3,4-dichlorophenyl)butyl]-N-methylbenzamide (compound example 115);
N-[(2S)-4-{4-[cyclohexyl(propionyl)amino]piperidin-1-yl}-2-(3,4-dichlorophenyl)butyl]-N-methyl-benzamide (compound example 116);
N-[(2S)-4-{4-[cyclohexyl(cyclopropylcarbonyl)-amino]piperidin-1-yl}-2-(3,4-dichlorophenyl)butyl]-N-methylbenzamide (compound example 117);
N-[(2S)-4-{4-[cyclohexyl(cyclohexylcarbonyl)amino]piperidin-1-yl}-2-(3,4-dichlorophenyl)butyl]-N-methylbenzamide (compound example 118);
N-[(2S)-4-{4-[benzoyl(cyclohexyl)amino]piperidin-1-yl}-2-(3,4-dichlorophenyl)butyl]-N-methyl-benzamide (compound example 119);
N-[(2S)-2-(3,4-dichlorophenyl)-4-{4-[(1-methyl-piperidin-4-yl)(propionyl)amino]piperidin-1-yl}butyl]-N-methylbenzamide (compound example 120);
N-[(2S)-4-{4-[(cyclopropylcarbonyl)(1-methylpiperidin-4-yl)amino]-piperidin-1-yl}-2-(3,4-dichlorophenyl)butyl]-N-methylbenzamide (compound example 121);
N-[(2S)-4-{4-[(cyclohexylcarbonyl)(1-methylpiperidin-4-yl)amino]piperidin-1-yl}-2-(3,4-dichlorophenyl)butyl]-N-methylbenzamide (compound example 122); and
N-{1-[(3S)-4-[benzoyl(methyl)amino]-3-(3,4-dichlorophenyl)butyl]piperidin-4-yl}-N-(1-methylpiperidin-4-yl)benzamide (compound example 123).

### EXAMPLE 37

### Preparation of Starting Materials (Compound III)

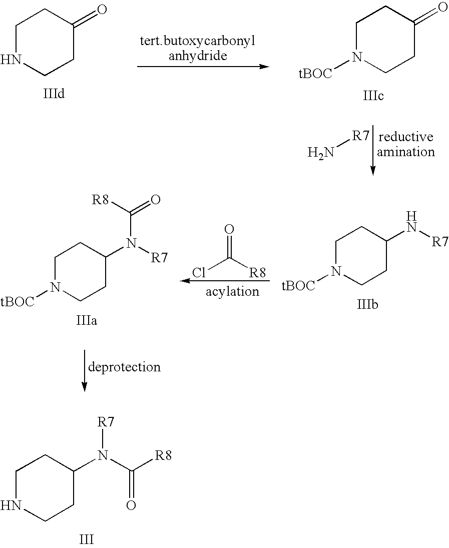

Commercially available compound IIId is reacted with tertiary butoxycarbonyl anhydride to deliver compound IIIc which is then is transformed to intermediate IIIb under conditions of reductive amination. Intermediate IIIb is acylated to the corresponding amide IIIa. Amide IIIa is then deprotected to yield intermediate III.

### EXAMPLE 38

### Preparation of N-(cyclopropylmethyl)-N-piperidin-4-ylpropanamide

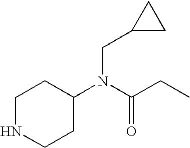

To a mixture of 1 g (5 mmole) of tert-butyl 4-oxopiperidine-1-carboxylate (compound IIIc), 0.39 ml (4.5 mmole) of 1-cyclopropylmethanamine and 0.56 g (6.8 mmmol) sodium acetate in 20 ml THF at room temperature under stirring were added 0.44 ml (7.75 mmole) acetic acid. After 1 hour stirring 1.93 g (9.1 mmole) of sodium triacetoxyborohydride were added to this mixture. After stirring for further 15 h the mixture was concentrated under vacuum to dryness and re-dissolved in 50 ml of ether. The organic solution was extracted three times with 30 ml of 0.1 n aqueous HCl. The combined water layers were alkalized by aqueous sodium hydroxide and then extracted three times with 30 ml of ether. The combined organic phase was dried on sodium sulfate, filtered and concentrated to dryness under vacuum to give 1.1 g crude tert-butyl 4-[(cyclopropylmethyl)amino]piperidine-1-carboxylate (IIIb).

To a 5° C. cold solution of 1.1 g (4.3 mmole) of crude crude tert-butyl 4-[(cyclopropylmethyl)-amino]piperidine-1-carboxylate (compound IIIb) and 1.13 ml (6.5 mmole) N-ethyl-diisopropylamine in 25 ml dichloromethane, 0.45 ml (5.2 mmole) propanoyl chloride in 3 ml dichloromethane were added. After stirring for 2 hours the mixture was evaporated and redissolved in 50 ml ether. The organic phase was sequentially washed 2 times with 30 ml water, 2 times with 20 ml of 0.1 N aqueous sodium hydroxide, with 30 ml of 0.1 N aqueous hydrochloric acid, dried on sodium sulfate and concentrated to give 1.1 g of crude tert-butyl 4-[(cyclopropylmethyl)(propionyl)amino]cyclohexane-carboxylate (compound IIIa).

The solution of 1.1 g of tert-butyl 4-[(cyclopropylmethyl)(propionyl)amino]-piperidine-1-carboxylate (compound IIIa) in 20 ml 4M hydrogen chloride in dioxane and 5 ml ethanol was stirred at room temperature for 15 hours, then evaporated and re-dissolved in 50 ml dichloromethane and sequentially washed 3 times with 20 ml of aqueous potassium carbonate (10%), with 30 ml water, dried on sodium sulfate and concentrated under vacuum to yield 0.45 g of N-(cyclopropylmethyl)-N-piperidin-4-ylpropanamide (compound III) as colorless oil.

The preparation of the required series of 4-substituted piperidines (see above) was performed according to the described process.

The foregoing description and the preceeding and following examples have been set forth merely to illustrate the invention and are not intended to be limiting. Since modifications of the described embodiments incorporating the spirit and substance of the invention may occur to persons skilled in the art, the invention should be construed broadly to include all variations within the scope of the appended claims and equivalents thereof.

The compounds of formula I listed in Table 15 below were prepared according to the process described in the above examples or according to processes analogous thereto.

TABLE 15
Compounds of Formula I

| Ex. | R¹ | R² | R³ | R⁴ | X | R⁵ | R6 | R7 | R8 | R9 | R10 | m |
|---|---|---|---|---|---|---|---|---|---|---|---|---|
| 1 | CH₃ | 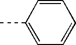 | Cl | Cl | CR6 | NR7R8 | (CO)ₘNR9N10 | 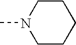 | | CH₃ | CH₃ | 1 |
| 2 | CH₃ | 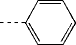 | Cl | Cl | CR6 | NR7R8 | (CO)ₘNR9N10 | 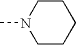 | | CH₃ | H | 1 |
| 3 | CH₃ | 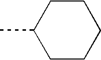 | Cl | Cl | CR6 | NR7R8 | (CO)ₘNR9N10 | 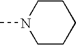 | | CH₃ | CH₃ | 1 |
| 4 | CH₃ | 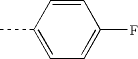 | Cl | Cl | CR6 | NR7R8 | (CO)ₘNR9N10 | 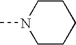 | | CH₃ | CH₃ | 1 |
| 5 | CH₃ | 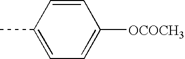 | Cl | Cl | CR6 | NR7R8 | (CO)ₘNR9N10 | 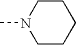 | | CH₃ | CH₃ | 1 |
| 6 | CH₃ | 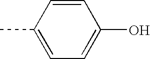 | Cl | Cl | CR6 | NR7R8 | (CO)ₘNR9N10 | 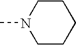 | | CH₃ | CH₃ | 1 |
| 7 | CH₃ | 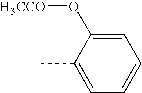 | Cl | Cl | CR6 | NR7R8 | (CO)ₘNR9N10 | 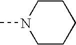 | | CH₃ | CH₃ | 1 |
| 8 | CH₃ | 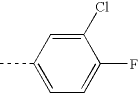 | Cl | Cl | CR6 | NR7R8 | (CO)ₘNR9N10 | 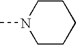 | | CH₃ | CH₃ | 1 |
| 9 | CH₃ | 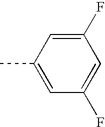 | Cl | Cl | CR6 | NR7R8 | (CO)ₘNR9N10 | 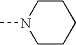 | | CH₃ | CH₃ | 1 |
| 10 | CH₃ | 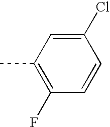 | Cl | Cl | CR6 | NR7R8 | (CO)ₘNR9N10 | 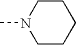 | | CH₃ | CH₃ | 1 |
| 11 | CH₃ | 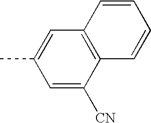 | Cl | Cl | CR6 | NR7R8 | (CO)ₘNR9N10 | 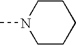 | | CH₃ | CH₃ | 1 |
| 12 | CH₃ | 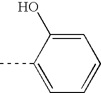 | Cl | Cl | CR6 | NR7R8 | (CO)ₘNR9N10 | 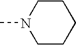 | | CH₃ | CH₃ | 1 |
| 13 | CH₃ | 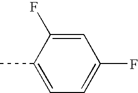 | Cl | Cl | CR6 | NR7R8 | (CO)ₘNR9N10 | 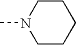 | | CH₃ | CH₃ | 1 |
| 14 | CH₃ | 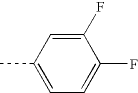 | Cl | Cl | CR6 | NR7R8 | (CO)ₘNR9N10 | 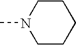 | | CH₃ | CH₃ | 1 |
| 15 | CH₃ | 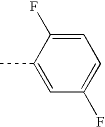 | Cl | Cl | CR6 | NR7R8 | (CO)ₘNR9N10 | 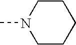 | | CH₃ | CH₃ | 1 |
| 16 | CH₃ | 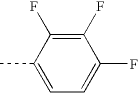 | Cl | Cl | CR6 | NR7R8 | (CO)ₘNR9N10 | 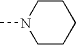 | | CH₃ | CH₃ | 1 |
| 17 | CH₃ | 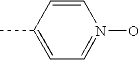 | Cl | Cl | CR6 | NR7R8 | (CO)ₘNR9N10 | 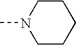 | | CH₃ | CH₃ | 1 |
| 18 | CH₃ | 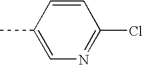 | Cl | Cl | CR6 | NR7R8 | (CO)ₘNR9N10 | 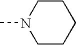 | | CH₃ | CH₃ | 1 |
| 19 | CH₃ | 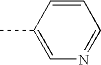 | Cl | Cl | CR6 | NR7R8 | (CO)ₘNR9N10 | 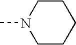 | | CH₃ | CH₃ | 1 |
| 20 | CH₃ | 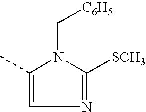 | Cl | Cl | CR6 | NR7R8 | (CO)ₘNR9N10 | 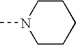 | | CH₃ | CH₃ | 1 |
| 21 | CH₃ | 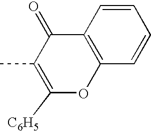 | Cl | Cl | CR6 | NR7R8 | (CO)ₘNR9N10 | 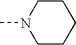 | | CH₃ | CH₃ | 1 |
| 22 | CH₃ | 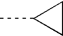 | Cl | Cl | CR6 | NR7R8 | (CO)ₘNR9N10 | 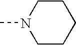 | | CH₃ | CH₃ | 1 |
| 23 | CH₃ | 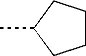 | Cl | Cl | CR6 | NR7R8 | (CO)ₘNR9N10 | 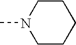 | | CH₃ | CH₃ | 1 |
| 24 | CH₃ | 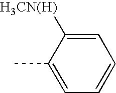 | Cl | Cl | CR6 | NR7R8 | (CO)ₘNR9N10 | 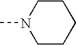 | | CH₃ | CH₃ | 1 |
| 25 | CH₃ | 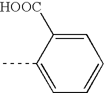 | Cl | Cl | CR6 | NR7R8 | (CO)ₘNR9N10 | 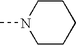 | | CH₃ | CH₃ | 1 |
| 26 | CH₃ | 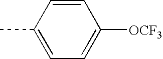 | Cl | Cl | CR6 | NR7R8 | (CO)ₘNR9N10 | 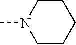 | | CH₃ | CH₃ | 1 |
| 27 | CH₃ | 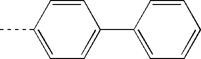 | Cl | Cl | CR6 | NR7R8 | (CO)ₘNR9N10 | 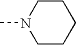 | | CH₃ | CH₃ | 1 |
| 28 | CH₃ | 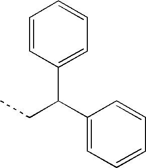 | Cl | Cl | CR6 | NR7R8 | (CO)ₘNR9N10 | 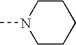 | | CH₃ | CH₃ | 1 |
| 29 | CH₃ | 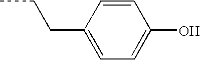 | Cl | Cl | CR6 | NR7R8 | (CO)ₘNR9N10 | 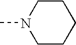 | | CH₃ | CH₃ | 1 |
| 30 | CH₃ | 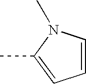 | Cl | Cl | CR6 | NR7R8 | (CO)ₘNR9N10 | 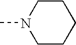 | | CH₃ | CH₃ | 1 |
| 31 | CH₃ | 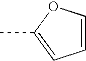 | Cl | Cl | CR6 | NR7R8 | (CO)ₘNR9N10 | 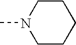 | | CH₃ | CH₃ | 1 |
| 32 | CH₃ | 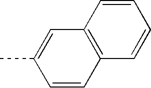 | Cl | Cl | CR6 | NR7R8 | (CO)ₘNR9N10 | 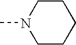 | | CH₃ | CH₃ | 1 |
| 33 | CH₃ | 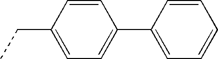 | Cl | Cl | CR6 | NR7R8 | (CO)ₘNR9N10 | 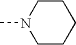 | | CH₃ | CH₃ | 1 |
| 34 | CH₃ | 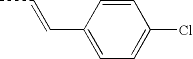 | Cl | Cl | CR6 | NR7R8 | (CO)ₘNR9N10 | 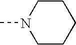 | | CH₃ | CH₃ | 1 |
| 35 | CH₃ | 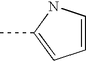 | Cl | Cl | CR6 | NR7R8 | (CO)ₘNR9N10 | 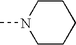 | | CH₃ | CH₃ | 1 |
| 36 | CH₃ | 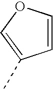 | Cl | Cl | CR6 | NR7R8 | (CO)ₘNR9N10 | 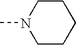 | | CH₃ | CH₃ | 1 |
| 37 | CH₃ | 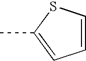 | Cl | Cl | CR6 | NR7R8 | (CO)ₘNR9N10 | 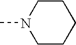 | | CH₃ | CH₃ | 1 |
| 38 | CH₃ | 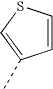 | Cl | Cl | CR6 | NR7R8 | (CO)ₘNR9N10 | 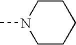 | | CH₃ | CH₃ | 1 |
| 39 | CH₃ | 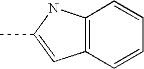 | Cl | Cl | CR6 | NR7R8 | (CO)ₘNR9N10 | 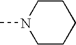 | | CH₃ | CH₃ | 1 |
| 40 | CH₃ | 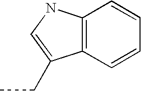 | Cl | Cl | CR6 | NR7R8 | (CO)ₘNR9N10 | 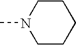 | | CH₃ | CH₃ | 1 |
| 41 | CH₃ | 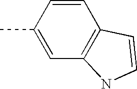 | Cl | Cl | CR6 | NR7R8 | (CO)ₘNR9N10 | 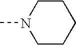 | | CH₃ | CH₃ | 1 |
| 42 | CH₃ | 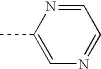 | Cl | Cl | CR6 | NR7R8 | (CO)ₘNR9N10 | 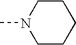 | | CH₃ | CH₃ | 1 |
| 43 | CH₃ | 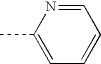 | Cl | Cl | CR6 | NR7R8 | (CO)ₘNR9N10 | 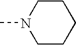 | | CH₃ | CH₃ | 1 |
| 44 | CH₃ | 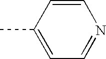 | Cl | Cl | CR6 | NR7R8 | (CO)ₘNR9N10 | 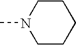 | | CH₃ | CH₃ | 1 |
| 45 | CH₃ | 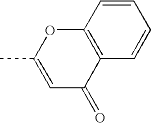 | Cl | Cl | CR6 | NR7R8 | (CO)ₘNR9N10 | 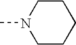 | | CH₃ | CH₃ | 1 |
| 46 | CH₃ | 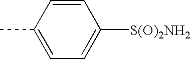 | Cl | Cl | CR6 | NR7R8 | (CO)ₘNR9N10 | 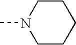 | | CH₃ | CH₃ | 1 |
| 47 | CH₃ | 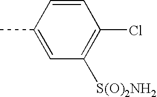 | Cl | Cl | CR6 | NR7R8 | (CO)ₘNR9N10 | 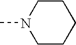 | | CH₃ | CH₃ | 1 |
| 48 | CH₃ | 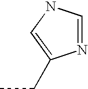 | Cl | Cl | CR6 | NR7R8 | (CO)ₘNR9N10 | 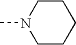 | | CH₃ | CH₃ | 1 |
| 49 | CH₃ | 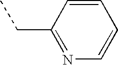 | Cl | Cl | CR6 | NR7R8 | (CO)ₘNR9N10 | 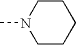 | | CH₃ | CH₃ | 1 |
| 50 | CH₃ | 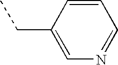 | Cl | Cl | CR6 | NR7R8 | (CO)ₘNR9N10 | 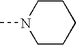 | | CH₃ | CH₃ | 1 |
| 51 | CH₃ | 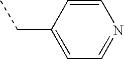 | Cl | Cl | CR6 | NR7R8 | (CO)ₘNR9N10 | 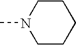 | | CH₃ | CH₃ | 1 |
| 52 | CH₃ | 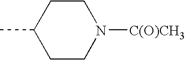 | Cl | Cl | CR6 | NR7R8 | (CO)ₘNR9N10 | 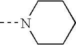 | | CH₃ | CH₃ | 1 |
| 53 | CH₃ | 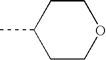 | Cl | Cl | CR6 | NR7R8 | (CO)ₘNR9N10 | 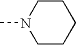 | | CH₃ | CH₃ | 1 |
| 54 | CH₃ | 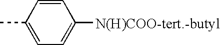 | Cl | Cl | CR6 | NR7R8 | (CO)ₘNR9N10 | 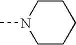 | | CH₃ | CH₃ | 1 |
| 55 | CH₃ | 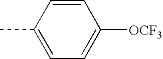 | Cl | Cl | CR6 | NR7R8 | (CO)ₘNR9N10 | 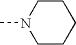 | | CH₃ | CH₃ | 1 |
| 56 | CH₃ | 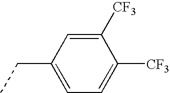 | Cl | Cl | CR6 | NR7R8 | (CO)ₘNR9N10 | 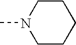 | | CH₃ | CH₃ | 1 |
| 57 | CH₃ | 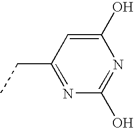 | Cl | Cl | CR6 | NR7R8 | (CO)ₘNR9N10 | 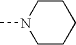 | | CH₃ | CH₃ | 1 |
| 58 | CH₃ | 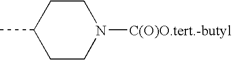 | Cl | Cl | CR6 | NR7R8 | (CO)ₘNR9N10 | 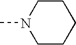 | | CH₃ | CH₃ | 1 |
| 59 | CH₃ | 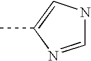 | Cl | Cl | CR6 | NR7R8 | (CO)ₘNR9N10 | 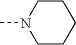 | | CH₃ | CH₃ | 1 |
| 60 | CH₃ | 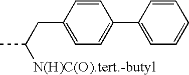 | Cl | Cl | CR6 | NR7R8 | (CO)ₘNR9N10 | 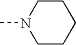 | | CH₃ | CH₃ | 1 |
| 61 | CH₃ | 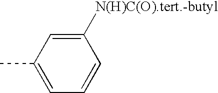 | Cl | Cl | CR6 | NR7R8 | (CO)ₘNR9N10 | 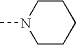 | | CH₃ | CH₃ | 1 |
| 62 | CH₃ | 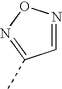 | Cl | Cl | CR6 | NR7R8 | (CO)ₘNR9N10 | 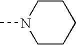 | | CH₃ | CH₃ | 1 |
| 63 | CH₃ | 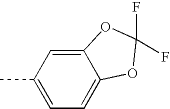 | Cl | Cl | CR6 | NR7R8 | (CO)ₘNR9N10 | 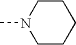 | | CH₃ | CH₃ | 1 |
| 64 | CH₃ | 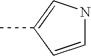 | Cl | Cl | CR6 | NR7R8 | (CO)ₘNR9N10 | 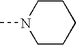 | | CH₃ | CH₃ | 1 |
| 65 | CH₃ | 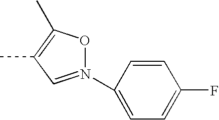 | Cl | Cl | CR6 | NR7R8 | (CO)ₘNR9N10 | 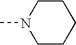 | | CH₃ | CH₃ | 1 |
| 66 | CH₃ | 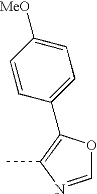 | Cl | Cl | CR6 | NR7R8 | (CO)ₘNR9N10 | 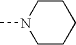 | | CH₃ | CH₃ | 1 |
| 67 | CH₃ | 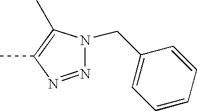 | Cl | Cl | CR6 | NR7R8 | (CO)ₘNR9N10 | 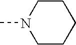 | | CH₃ | CH₃ | 1 |
| 68 | CH₃ | 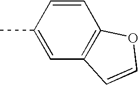 | Cl | Cl | CR6 | NR7R8 | (CO)ₘNR9N10 | 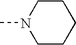 | | CH₃ | CH₃ | 1 |
| 69 | CH₃ | 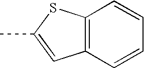 | Cl | Cl | CR6 | NR7R8 | (CO)ₘNR9N10 | 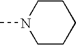 | | CH₃ | CH₃ | 1 |
| 70 | CH₃ | 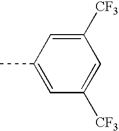 | Cl | Cl | CR6 | NR7R8 | (CO)ₘNR9N10 | 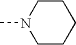 | | CH₃ | CH₃ | 1 |
| 71 | CH₃ | 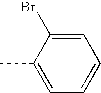 | Cl | Cl | CR6 | NR7R8 | (CO)ₘNR9N10 | 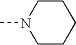 | | CH₃ | CH₃ | 1 |
| 72 | CH₃ | 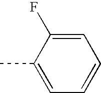 | Cl | Cl | CR6 | NR7R8 | (CO)ₘNR9N10 | 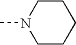 | | CH₃ | CH₃ | 1 |
| 73 | CH₃ | 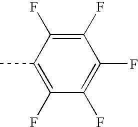 | Cl | Cl | CR6 | NR7R8 | (CO)ₘNR9N10 | 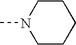 | | CH₃ | CH₃ | 1 |
| 74 | CH₃ | 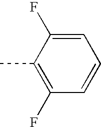 | Cl | Cl | CR6 | NR7R8 | (CO)ₘNR9N10 | 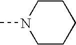 | | CH₃ | CH₃ | 1 |
| 75 | CH₃ | 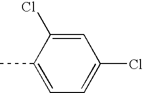 | Cl | Cl | CR6 | NR7R8 | (CO)ₘNR9N10 | 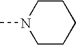 | | CH₃ | CH₃ | 1 |
| 76 | CH₃ | 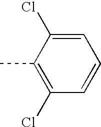 | Cl | Cl | CR6 | NR7R8 | (CO)ₘNR9N10 | 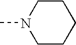 | | CH₃ | CH₃ | 1 |
| 77 | CH₃ | 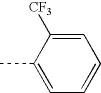 | Cl | Cl | CR6 | NR7R8 | (CO)ₘNR9N10 | 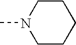 | | CH₃ | CH₃ | 1 |
| 78 | CH₃ | 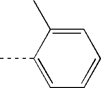 | Cl | Cl | CR6 | NR7R8 | (CO)ₘNR9N10 | 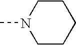 | | CH₃ | CH₃ | 1 |
| 79 | CH₃ | 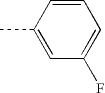 | Cl | Cl | CR6 | NR7R8 | (CO)ₘNR9N10 | 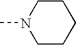 | | CH₃ | CH₃ | 1 |
| 80 | CH₃ | 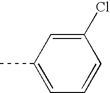 | Cl | Cl | CR6 | NR7R8 | (CO)ₘNR9N10 | 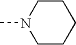 | | CH₃ | CH₃ | 1 |
| 81 | CH₃ | 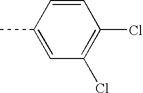 | Cl | Cl | CR6 | NR7R8 | (CO)ₘNR9N10 | 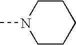 | | CH₃ | CH₃ | 1 |
| 82 | CH₃ | 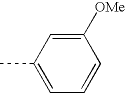 | Cl | Cl | CR6 | NR7R8 | (CO)ₘNR9N10 | 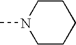 | | CH₃ | CH₃ | 1 |
| 83 | CH₃ | 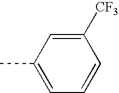 | Cl | Cl | CR6 | NR7R8 | (CO)ₘNR9N10 | 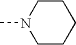 | | CH₃ | CH₃ | 1 |
| 84 | CH₃ | 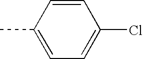 | Cl | Cl | CR6 | NR7R8 | (CO)ₘNR9N10 | 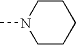 | | CH₃ | CH₃ | 1 |
| 85 | CH₃ | 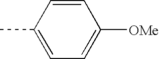 | Cl | Cl | CR6 | NR7R8 | (CO)ₘNR9N10 | 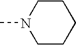 | | CH₃ | CH₃ | 1 |
| 86 | CH₃ | 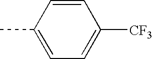 | Cl | Cl | CR6 | NR7R8 | (CO)ₘNR9N10 | 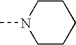 | | CH₃ | CH₃ | 1 |
| 87 | CH₃ | 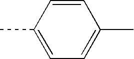 | Cl | Cl | CR6 | NR7R8 | (CO)ₘNR9N10 | 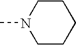 | | CH₃ | CH₃ | 1 |
| 88 | CH₃ | 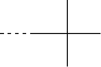 | Cl | Cl | CR6 | NR7R8 | (CO)ₘNR9N10 | 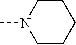 | | CH₃ | CH₃ | 1 |
| 89 | CH₃ | 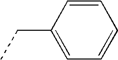 | Cl | Cl | CR6 | NR7R8 | (CO)ₘNR9N10 | 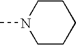 | | CH₃ | CH₃ | 1 |
| 90 | CH₃ | 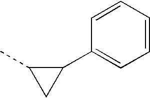 | Cl | Cl | CR6 | NR7R8 | (CO)ₘNR9N10 | 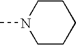 | | CH₃ | CH₃ | 1 |
| 91 | CH₃ | 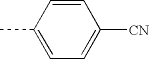 | Cl | Cl | CR6 | NR7R8 | (CO)ₘNR9N10 | 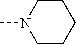 | | CH₃ | CH₃ | 1 |
| 92 | CH₃ | 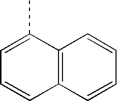 | Cl | Cl | CR6 | NR7R8 | (CO)ₘNR9N10 | 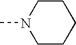 | | CH₃ | CH₃ | 1 |
| 93 | CH₃ | 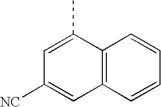 | Cl | Cl | CR6 | NR7R8 | (CO)ₘNR9N10 | 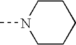 | | CH₃ | H | 1 |
| 94 | CH₃ | 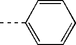 | H | H | CR6 | NR7R8 | (CO)ₘNR9N10 | 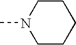 | | CH₃ | CH₃ | 1 |
| 95 | CH₃ | 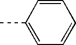 | Cl | Cl | CR6 | NR7R8 | (CO)ₘNR9N10 | 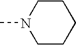 | | C₂H₅ | CH₃ | 1 |
| 96 | CH₃ | 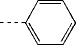 | Cl | Cl | N | 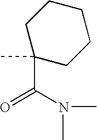 | — | — | | — | — | — |
| 97 | CH₃ | 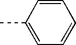 | Cl | Cl | CR6 | NR7R8 | H | 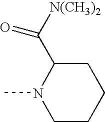 | | — | — | — |
| 98 | CH₃ | 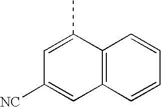 | Cl | Cl | CR6 | NR7R8 | (CO)ₘNR9N10 | 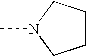 | | CH₃ | CH₃ | 1 |
| 99 | CH₃ | 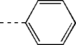 | Cl | Cl | CR6 | NR7R8 | (CO)ₘNR9N10 | 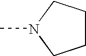 | | CH₃ | CH₃ | 1 |
| 100 | CH₃ | 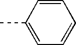 | Cl | Cl | CR6 | NR7R8 | H | 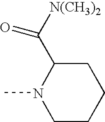 | | — | — | — |
| 101 | CH₃ | 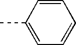 | Cl | Cl | CR6 | NR7R8 | (CO)ₘNR9N10 | — | | CH₃ | CH₃ | 1 |
| 102 | CH₃ | 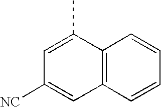 | Cl | Cl | CR6 | NR7R8 | (CO)ₘNR9N10 | 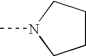 | | 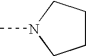 | | 1 |
| 103 | CH₃ | 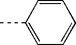 | Cl | Cl | CR6 | NR7R8 | (CO)ₘNR9N10 | 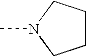 | | 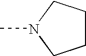 | | 1 |
| 104 | CH₃ | 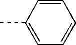 | Cl | Cl | CR6 | NR7R8 | (CO)ₘNR9N10 | 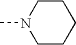 | | 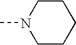 | | 1 |
| 105 | CH₃ | 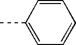 | Cl | Cl | CR6 | NR7R8 | H | 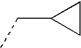 | C(O)C₂H₅ | — | — | — |
| 106 | CH₃ | 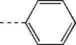 | Cl | Cl | CR6 | NR7R8 | H | i—propyl | C(O)C₂H₅ | — | — | — |
| 107 | CH₃ | 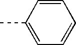 | Cl | Cl | CR6 | NR7R8 | H | 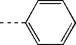 | C(O)C₂H₅ | — | — | — |
| 108 | CH₃ | 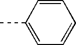 | Cl | Cl | CR6 | NR7R8 | H | n—butyl | C(O)C₂H₅ | — | — | — |
| 109 | CH₃ | 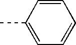 | Cl | Cl | CR6 | NR7R8 | (CO)ₘNR9N10 | n—butyl | C(O)cyclo- propyl | — | — | — |
| 110 | CH₃ | 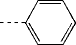 | Cl | Cl | CR6 | NR7R8 | (CO)ₘNR9N10 | n—butyl | C(O)cyclo- hexyl | — | — | — |
| 111 | CH₃ | 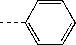 | Cl | Cl | CR6 | NR7R8 | (CO)ₘNR9N10 | n—butyl | C(O)phenyl | — | — | — |
| 112 | CH₃ | 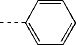 | Cl | Cl | CR6 | NR7R8 | (CO)ₘNR9N10 | (CH₂)₄OCH₃ | C(O)ethyl | — | — | — |
| 113 | CH₃ | 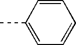 | Cl | Cl | CR6 | NR7R8 | (CO)ₘNR9N10 | C(O)cyclo- propyl | — | — | — | |
| 114 | CH₃ | 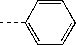 | Cl | Cl | CR6 | NR7R8 | (CO)ₘNR9N10 | (CH₂)₄OCH₃ | C(O)cyclo- hexyl | — | — | — |
| 115 | CH₃ | 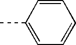 | Cl | Cl | CR6 | NR7R8 | (CO)ₘNR9N10 | (CH₂)₄OCH₃ | C(O)phenyl | — | — | — |
| 116 | CH₃ | 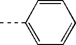 | Cl | Cl | CR6 | NR7R8 | (CO)ₘNR9N10 | Cyclo—hexyl | C(O)ethyl | — | — | — |
| 117 | CH₃ | 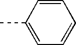 | Cl | Cl | CR6 | NR7R8 | (CO)ₘNR9N10 | Cyclo—hexyl | C(O)cyclo- propyl | — | — | — |
| 118 | CH₃ | 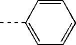 | Cl | Cl | CR6 | NR7R8 | (CO)ₘNR9N10 | Cyclo—hexyl | C(O)cyclo- hexyl | — | — | — |
| 119 | CH₃ | 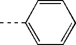 | Cl | Cl | CR6 | NR7R8 | (CO)ₘNR9N10 | Cyclo—hexyl | C(O)phenyl | — | — | — |
| 120 | CH₃ | 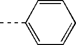 | Cl | Cl | CR6 | NR7R8 | (CO)ₘNR9N10 | 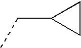 | C(O)ethyl | — | — | — |
| 121 | CH₃ | 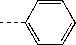 | Cl | Cl | CR6 | NR7R8 | (CO)ₘNR9N10 | 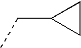 | C(O)cyclo- propyl | — | — | — |
| 122 | CH₃ | 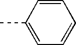 | Cl | Cl | CR6 | NR7R8 | (CO)ₘNR9N10 | 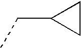 | C(O)cyclo- hexyl | — | — | — |
| 123 | CH₃ | 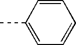 | Cl | Cl | CR6 | NR7R8 | (CO)ₘNR9N10 | 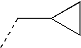 | C(O)phenyl | | | |
| 124 | CH₃ | 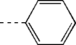 | Cl | Cl | CR6 | NR7R8 | (CO)ₘNR9N10 | 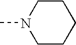 | | 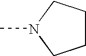 | | 1 |
| 125 | CH₃ | 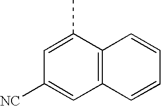 | Cl | Cl | CR6 | NR7R8 | (CO)ₘNR9N10 | 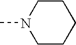 | | 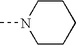 | | 1 |
| 126 | CH₃ | 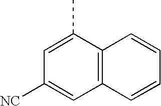 | Cl | Cl | CR6 | NR7R8 | (CO)ₘNR9N10 | 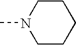 | | 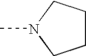 | | 1 |
| 127 | CH₃ | 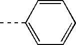 | Cl | Cl | CR6 | NR7R8 | (CO)ₘNR9N10 | CH₃ | CH₃ | CH₃ | CH₃ | 1 |

| 128 | CH₃ | 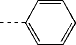 | Cl | Cl | CR6 | NR7R8 | (CO)ₘNR9N10 | 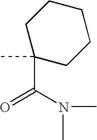 | 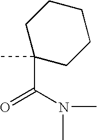 | | 1 |
|---|---|---|---|---|---|---|---|---|---|---|---|
| 129 | CH₃ | 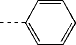 | Cl | Cl | CR6 | NR7R8 | (CO)ₘNR9N10 | 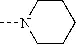 | C₂H₅ | C₂H₅ | CH₃ |
| 130 | CH₃ | 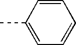 | Cl | Cl | CR6 | NR7R8 | (CO)ₘNR9N10 | 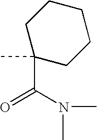 | CH₃ | CH₃ | 1 |

Table 16 contains analytical data from mass spectroscopy indicating the retention time in relation to the molecular weight observed.

TABLE 16
MS data and retention time

| Example | Percent | Ret. time | Mol. Weight Batch |
|---|---|---|---|
| 1 | 100 | 5.43 | 573.6048 |
| 3 | 100 | 5.74 | 579.6522 |
| 4 | 100 | 5.47 | 591.5949 |
| 14 | 100 | 5.58 | 609.585 |
| 19 | 100 | 4.86 | 574.5929 |
| 20 | 96.44 | 5.65 | 699.7872 |
| 21 | 92.45 | 5.77 | 717.7334 |
| 22 | 100 | 5.23 | 537.5718 |
| 23 | 100 | 5.65 | 565.6254 |
| 24 | 100 | 5.59 | 602.6465 |
| 25 | 100 | 5.29 | 617.6138 |
| 26 | 99.43 | 5.93 | 657.6009 |
| 27 | 100 | 5.99 | 649.7024 |
| 28 | 100 | 6.25 | 677.756 |
| 29 | 100 | 5.26 | 617.6574 |
| 30 | 100 | 5.42 | 576.6087 |
| 31 | 100 | 5.27 | 563.566 |
| 32 | 100 | 5.75 | 623.6646 |
| 33 | 100 | 6.06 | 663.7292 |
| 34 | 100 | 5.87 | 634.0877 |
| 35 | 100 | 5.36 | 562.5819 |
| 36 | 100 | 5.25 | 563.566 |
| 37 | 100 | 5.43 | 579.633 |
| 38 | 100 | 5.36 | 579.633 |
| 39 | 100 | 5.74 | 612.6417 |
| 40 | 100 | 5.52 | 626.6685 |
| 41 | 100 | 5.3 | 612.6417 |
| 42 | 98.54 | 4.91 | 575.581 |
| 43 | 100 | 5.03 | 574.5929 |
| 44 | 100 | 4.84 | 574.5929 |
| 45 | 100 | 5.27 | 641.6358 |
| 46 | 100 | 4.96 | 652.6837 |
| 47 | 100 | 5.13 | 687.1288 |
| 48 | 100 | 4.51 | 577.5968 |
| 49 | 100 | 5.06 | 588.6197 |
| 50 | 100 | 4.92 | 588.6197 |
| 51 | 100 | 4.85 | 588.6197 |
| 52 | 100 | 4.79 | 622.6771 |
| 53 | 99.7 | 5.01 | 581.6244 |
| 54 | 100 | 5.8 | 688.7359 |
| 55 | 100 | 5.9 | 657.6009 |
| 56 | 100 | 6.3 | 723.6258 |
| 57 | 100 | 4.59 | 621.6058 |
| 58 | 100 | 5.7 | 680.7565 |
| 59 | 98.64 | 4.7 | 563.57 |
| 60 | 100 | 6.57 | 792.8871 |
| 61 | 100 | 5.79 | 688.7359 |
| 62 | 100 | 5.1 | 565.5422 |
| 63 | 100 | 5.79 | 653.594 |
| 64 | 100 | 5.06 | 562.5819 |
| 65 | 100 | 5.7 | 672.6686 |
| 66 | 100 | 5.47 | 670.6775 |
| 67 | 100 | 5.52 | 668.7093 |
| 68 | 100 | 5.49 | 613.6258 |
| 69 | 100 | 6.05 | 643.7196 |
| 70 | 100 | 6.22 | 709.599 |
| 71 | 100 | 5.71 | 652.5009 |
| 72 | 99.84 | 5.5 | 591.5949 |
| 73 | 100 | 5.95 | 663.5553 |
| 74 | 92.69 | 5.4 | 609.585 |
| 75 | 98.97 | 5.92 | 642.495 |
| 76 | 92.88 | 5.61 | 642.495 |
| 77 | 100 | 5.8 | 641.6019 |
| 78 | 100 | 5.56 | 587.6316 |
| 79 | 100 | 5.5 | 591.5949 |
| 80 | 100 | 5.63 | 608.0499 |
| 81 | 100 | 5.86 | 642.495 |
| 82 | 100 | 5.44 | 603.6306 |
| 83 | 100 | 5.77 | 641.6019 |
| 84 | 100 | 5.66 | 608.0499 |
| 85 | 100 | 5.41 | 603.6306 |
| 86 | 98.96 | 5.82 | 641.6019 |
| 87 | 100 | 5.6 | 587.6316 |
| 88 | 100 | 5.67 | 553.6144 |
| 89 | 100 | 5.57 | 587.6316 |
| 91 | 100 | 5.31 | 598.6149 |
| 92 | 100 | 5.7 | 623.6646 |

### EXAMPLE 39

### Capsules containing 1′-[4-(Benzoyl-methyl-amino)-3-(3,4-dichlorophenyl)-butyl]-[1,4′]bipiperidinyl-4′-carboxylic acid dimethylamide;

Capsules having the following composition per capsule were produced:

| | |
|---|---|
| 1′-[4-(Benzoyl-methyl-amino)-3-(3,4-dichlorophenyl)-butyl]- | 20 mg |
| [1,4′]bipiperidinyl-4′-carboxylic acid dimethylamide | |
| Corn starch | 60 mg |
| Lactose | 300 mg |
| Ethyl acetate | q.s. |

The active substance, the corn starch and the lactose were processed into a homogenous pasty mixture using ethyl acetate. The paste was ground and the resulting granules were placed on a suitable tray and dried at 45° C. in order to remove the solvent. The dried granules were passed through a crusher and mixed in a mixer with the further following auxiliaries:

| | |
|---|---|
| Talcum | 5 mg |
| Magnesium stearate | 5 mg |
| Corn starch | 9 mg |

and then filled into 400 mg capsules (=capsule size 0).

### EXAMPLE 40

### Capsules containing 1′-[4-(Benzoyl-methyl-amino)-3-(3,4-dichlorophenyl)-butyl]-[1,4′]bipiperidinyl-4′-carboxylic acid methylamide

Capsules having with the following composition per capsule were produced:

| | |
|---|---|
| 1′-[4-(Benzoyl-methyl-amino)-3-(3,4-dichlorophenyl)- | 20 mg |
| butyl]-[1,4′]bipiperidinyl-4′-carboxylic acid methylamide | |
| Corn starch | 60 mg |
| Lactose | 300 mg |
| Ethyl acetate | q.s. |

The active substance, the corn starch and the lactose were processed into a homogenous pasty mixture using ethyl acetate. The paste was ground and the resulting granules were placed on a suitable tray and dried at 45° C. in order to remove the solvent. The dried granules were passed through a crusher and mixed in a mixer with the further following auxiliaries:

| | |
|---|---|
| Talcum | 5 mg |
| Magnesium stearate | 5 mg |
| Corn starch | 9 mg |

and then poured into 400 mg capsules (=capsule size 0).

### EXAMPLE 41

### Capsules containing 1′-[4-(Cyclohexanecarbonyl-methyl-amino)-3-(3,4-dichlorophenyl)-butyl]-[1,4′]bipiperidinyl-4′-carboxylic acid dimethylamide

Capsules with the following composition per capsule were produced:

| | |
|---|---|
| 1′-[4-(Cyclohexanecarbonyl-methyl-amino)-3-(3,4- | 20 mg |
| dichlorophenyl)-butyl]-[1,4′]bipiperidinyl- | |
| 4′-carboxylic acid dimethylamide | |
| Corn starch | 60 mg |
| Lactose | 300 mg |
| Ethyl acetate | q.s. |

The active substance, the corn starch and the lactose were processed into a homogenous pasty mixture using ethyl acetate. The paste was ground and the resulting granules were placed on a suitable tray and dried at 45° C. in order to remove the solvent. The dried granules were passed through a crusher and mixed in a mixer with the further following auxiliaries:

| | |
|---|---|
| Talcum | 5 mg |
| Magnesium stearate | 5 mg |
| Corn starch | 9 mg |

and then poured into 400 mg capsules (=capsule size 0).

### EXAMPLE 42

### Capsules containing 1′-{3-(3,4-Dichlorophenyl)-4-[(4-fluoro-benzoyl)-methyl-amino]-butyl}-[1,4′]bipiperidinyl-4′-carboxylic acid dimethylamide

Capsules with the following composition per capsule were produced:

| | |
|---|---|
| 1′-{3-(3,4-Dichlorophenyl)-4-[(4-fluoro-benzoyl)- | 20 mg |
| methyl-amino]-butyl}-[1,4′]bipiperidinyl-4′- | |
| carboxylic acid dimethylamide | |
| Corn starch | 60 mg |
| Lactose | 300 mg |
| Ethyl acetate | q.s. |

The active substance, the corn starch and the lactose were processed into a homogenous pasty mixture using ethyl acetate. The paste was ground and the resulting granules were placed on a suitable tray and dried at 45° C. in order to remove the solvent. The dried granules were passed through a crusher and mixed in a mixer with the further following auxiliaries:

| | |
|---|---|
| Talcum | 5 mg |
| Magnesium stearate | 5 mg |
| Corn starch | 9 mg |

and then poured into 400 mg capsules (=capsule size 0).

What is claimed is:

1. A compound corresponding to formula I: 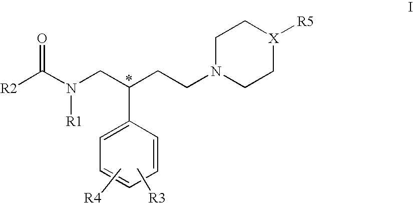
wherein
R1 is selected from the group consisting of alkyl and cycloalkyl;
R2 is selected from the group consisting of alkyl, cycloalkyl, aryl, alkylenearyl, alkenylenearyl, heteroaryl, and heterocyclic ring;
R3 and R4 are independently selected from the group consisting of hydrogen, halogen, hydroxyl, cyano, and carboxyalkyl;
X is selected from the group consisting of CR6 and nitrogen;
R5 is selected from the group consisting of alkyl optionally substituted with (CO)ₘNR9R10, cycloalkyl optionally substituted with (CO)ₘNR9R10, and NR7R8;
R6 is selected from the group consisting of hydrogen, alkyl, cycloalkyl, and (CO)ₘNR9R10;
R7 and R8 are independently selected from the group consisting of alkyl, cycloalkyl, aryl, alkylenearyl, alkyleneoxyalkyl, COalkyl, COaryl, or wherein R7 and R8 form together a 5- to 7-membered ring optionally containing an additional heteroatom, wherein such ring may be substituted by CONR9R10, and wherein in a 6-membered ring, none of the ring atoms is replaced by carbonyl;
R9 and R10 are independently selected from the group consisting of hydrogen, alkyl, cycloalkyl, aryl, alkylenearyl, alkyleneoxyalkyl, or wherein R9 and R10 form together a 5- to 7-membered ring optionally containing an additional heteroatom;
m is 0 or 1; or
a physiologically compatible salt thereof.

2. A compound according to claim 1, wherein R1 is methyl.

3. A compound according to claim 1, wherein R3 and R4 are independently selected from the group consisting of hydrogen, fluoro, and chloro.

4. A compound according to claim 1, wherein R3 and R4 are independently hydrogen or chloro.

5. A compound according to claim 1, wherein:
X is CR6,
R5 is NR7R8, and
R6 is (CO)ₘNR9R10 and m=1.

6. A compound according to claim 1, wherein:
X is N, and
R5 is cycloalkyl substituted with (CO)ₘNR9R10 and m=1.

7. A compound according to claim 1, wherein R7 and R8 together form a 6-membered ring or a 6-membered ring substituted by CONR9R10.

8. A compound according to claim 1, wherein R9 and R10 are each methyl, or wherein R9 and R10 together form a 6-membered ring or a 5-membered ring substituted by carbonyl.

9. A compound according to claim 1, wherein R2 is selected from the group consisting of:
C₁ to C₂₀ alkyl; C₃ to C₂₀ cycloalkyl; C₂ to C₂₀ alkenyl; 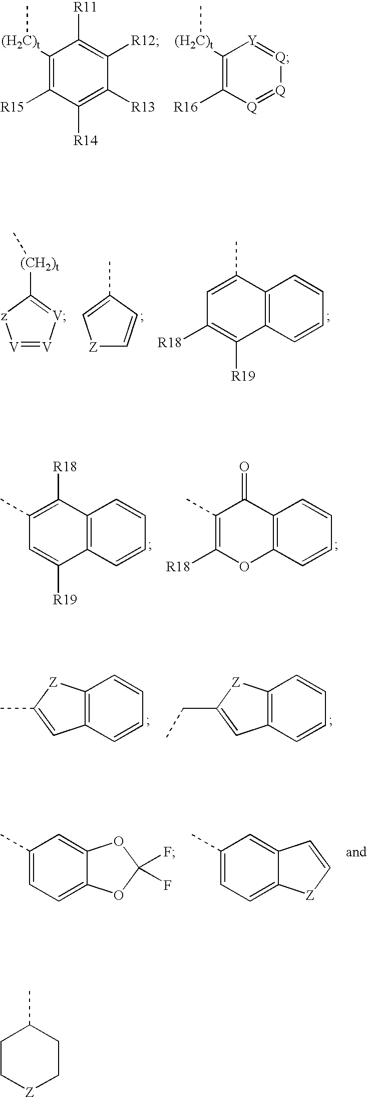
wherein
R11 to R16 are each independently selected from the group consisting of hydrogen, fluoro, chloro, bromo, hydroxyl, alkoxy, cyano, N(H)C(O)Oalkyl, aminoalkyl, dialkylamino, OCF₃, CF₃, carboxyalkyl, S(O)₂NH₂, phenyl, alkyl, and cycloalkyl;
R18 and R19 are each independently selected from the group consisting of hydrogen, cyano and aryl;
t is 0 or 1;
each Q is independently selected from the group consisting of CR11 and N;
Y is selected from the group consisting of CH, N and NO;
Z is selected from the group consisting of C-benzyl, NH, N-benzyl, N-alkyl, O and S;
each V is independently selected from the group consisting of N and CR17; and
R17 is selected from the group consisting of hydrogen, alkyl, cycloalkyl, aryl, and thioalkyl.

10. A compound according to claim wherein R5 is selected from the group consisting of:

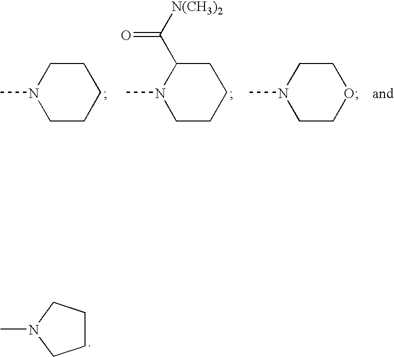

11. A compound according to claim 1, selected from the group consisting of:
1′-[4-(benzoyl-methyl-amino)-3-(3,4-dichlorophenyl)-butyl]-[1,4′]bipiperidinyl-4′-carboxylic acid dimethylamide;
1′-[4-(benzoyl-methyl-amino)-3-(3,4-dichlorophenyl)-butyl]-[1,4′]bipiperidinyl-4′-carboxylic acid methylamide;
1′-[4-(cyclohexanecarbonyl-methyl-amino)-3-(3,4-dichlorophenyl)-butyl]-[1,4′]bipiperidinyl-4′-carboxylic acid dimethylamide;
1′{-3-(3,4-dichlorophenyl)-4-[(4-fluoro-benzoyl)-methyl-amino]-butyl}-[1,4′]bipiperidinyl-4′-carboxylic acid dimethylamide;
acetic acid 4-{[2-(3,4-dichlorophenyl)-4-(4′-dimethylcarbamoyl-[1,4′]bi-piperidinyl-1′-yl)-butyl]-methyl-carbamoyl}-phenyl ester;
1′{-3-(3,4-dichlorophenyl)-4-[(4-hydroxy-benzoyl)-methyl-amino]-butyl}-[1,4′]bi-piperidinyl-4′-carboxylic acid dimethylamide;
acetic acid 2-{[2-(3,4-dichlorophenyl)-4-(4′-dimethylcarbamoyl-[1,4′]bi-piperidinyl-1′-yl)-butyl]-methyl-carbamoyl}-phenyl ester;
1′-[4-[(3-chloro-4-fluoro-benzoyl)-methyl-amino]-3-(3,4-dichlorophenyl)-butyl]-[1,4′]bipiperidinyl-4′-carboxylic acid dimethylamide;
1′-{3-(3,4-dichlorophenyl)-4-[(3,5-difluoro-benzoyl)-methyl-amino]-butyl}-[1,4′]bipiperidinyl-4′-carboxylic acid dimethylamide;
1′-[4-[(5-chloro-2-fluoro-benzoyl)-methyl-amino]-3-(3,4-dichlorophenyl)-butyl]-[1,4′]bipiperidinyl-4′-carboxylic acid dimethylamide;
1′-{3-(3,4-dichlorophenyl)-4-[methyl-(naphthalene-1-carbonyl-3-cyano)-amino]-butyl}-[1,4′]bipiperidinyl-4′-carboxylic acid dimethylamide;
1′-{3-(3,4-dichlorophenyl)-4-[(2-hydroxy-benzoyl)-methyl-amino]-butyl}-[1,4′]bipiperidinyl-4′-carboxylic acid dimethylamide;
1′-{3-(3,4-dichlorophenyl)-4-[(2,4-difluoro-benzoyl)-methyl-amino]-butyl}-[1,4′]bipiperidinyl-4′-carboxylic acid dimethylamide;
1′-{3-(3,4-dichlorophenyl)-4-[(3,4-difluoro-benzoyl)-methyl-amino]-butyl}-[1,4′]bipiperidinyl-4′-carboxylic acid dimethylamide;
1′-{3-(3,4-dichlorophenyl)-4-[(2,5-difluoro-benzoyl)-methyl-amino]-butyl}-[1,4′]bipiperidinyl-4′-carboxylic acid dimethylamide;
1′-{3-(3,4-dichlorophenyl)-4-[methyl-(2,3,4-trifluoro-benzoyl)-amino]-butyl}-[1,4′]bipiperidinyl-4′-carboxylic acid dimethylamide;
1′-{3-(3,4-dichlorophenyl)-4-[methyl-(1-oxy-pyridine-4-carbonyl)-amino]-butyl}-[1,4′]bipiperidinyl-4′-carboxylic acid dimethylamide;
1′-[4-[(6-chloro-pyridine-3-carbonyl)-methyl-amino]-3-(3,4-dichlorophenyl)-butyl]-[1,4′]bipiperidinyl-4′-carboxylic acid dimethylamide;
1′-{3-(3,4-dichlorophenyl)-4-[methyl-(pyridine-3-carbonyl)-amino]-butyl}-[1,4′]bipiperidinyl-4′-carboxylic acid dimethylamide;
1′-[4-[(3-benzyl-2-methylsulfanyl-3H-imidazole-4-carbonyl)-methyl-amino]-3-(3,4-dichlorophenyl)-butyl]-[1,4′]bipiperidinyl-4′-carboxylic acid dimethylamide;
1′-{3-(3,4-dichlorophenyl)-4-[methyl-(4-oxo-2-phenyl-4H-chromene-3-carbonyl)-amino]-butyl}-[1,4′]bipiperidinyl-4′-carboxylic acid dimethylamide;
1′-[4-(cyclopropanecarbonyl-methyl-amino)-3-(3,4-dichlorophenyl)-butyl]-[1,4′]bipiperidinyl-4′-carboxylic acid dimethylamide;
1′-[4-(cyclopentanecarbonyl-methyl-amino)-3-(3,4-dichlorophenyl)-butyl]-[1,4′]bipiperidinyl-4′-carboxylic acid dimethylamide;
1′-{3-(3,4-dichlorophenyl)-4-[methyl-(2-methylamino-benzoyl)-amino]-butyl}-[1,4′]bipiperidinyl-4′-carboxylic acid dimethylamide;
N-[2-(3,4-dichlorophenyl)-4-(4′-dimethylcarbamoyl-[1,4′]bipiperidinyl-1′-yl)-butyl]-N-methyl-phthalamic acid;
1′-{3-(3,4-dichlorophenyl)-4-[(4-methoxy-benzoyl)-methyl-amino]-butyl}-[1,4′]bipiperidinyl-4′-carboxylic acid dimethylamide;
1′-[4-[(biphenyl-4-carbonyl)-methyl-amino]-3-(3,4-dichlorophenyl)-butyl]-[1,4′]bipiperidinyl-4′-carboxylic acid dimethylamide;
1′-{3-(3,4-dichlorophenyl)-4-[(3,3-diphenyl-propionyl)-methyl-amino]-butyl}-[1,4′]bipiperidinyl-4′-carboxylic acid dimethylamide;
1′-(3-(3,4-dichlorophenyl)-4-{[3-(4-hydroxy-phenyl)-propionyl]-methyl-amino}-butyl)-[1,4′]bipiperidinyl-4′-carboxylic acid dimethylamide;
1′-{3-(3,4-dichlorophenyl)-4-[methyl-(1-methyl-1H-pyrrole-2-carbonyl)-amino]-butyl}-[1,4′]bipiperidinyl-4′-carboxylic acid dimethylamide;
1′-{3-(3,4-dichlorophenyl)-4-[(furan-2-carbonyl)-methyl-amino]-butyl}-[1,4′]bipiperidinyl-4′-carboxylic acid dimethylamide;
1′-{3-(3,4-dichlorophenyl)-4-[methyl-(naphthalene-2-carbonyl)-amino]-butyl}-[1,4′]bipiperidinyl-4′-carboxylic acid dimethylamide;
1′-[4-[(2-biphenyl-4-yl-acetyl)-methyl-amino]-3-(3,4-dichlorophenyl)-butyl]-[1,4′]bipiperidinyl-4′-carboxylic acid dimethylamide;
1′-[4-{[3-(4-chlorophenyl)-acryloyl]-methyl-amino}-3-(3,4-dichlorophenyl)-butyl]-[1,4′]bipiperidinyl-4′-carboxylic acid dimethylamide;
1′-{3-(3,4-dichlorophenyl)-4-[methyl-(1H-pyrrole-2-carbonyl)-amino]-butyl}-[1,4′]bipiperidinyl-4′-carboxylic acid dimethylamide;
1′-{3-(3,4-dichlorophenyl)-4-[(furan-2-carbonyl)-methyl-amino]-butyl}-[1,4′]bipiperidinyl-4′-carboxylic acid dimethylamide;
1′-{3-(3,4-dichlorophenyl)-4-[methyl-(thiophene-2-carbonyl)-amino]-butyl}-[1,4′]bipiperidinyl-4′-carboxylic acid dimethylamide;
1′-{3-(3,4-dichlorophenyl)-4-[methyl-(thiophene-3-carbonyl)-amino]-butyl}-[1,4′]bipiperidinyl-4′-carboxylic acid dimethylamide;
1′-{3-(3,4-dichlorophenyl)-4-[(1H-indole-3-carbonyl)-methyl-amino]-butyl}-[1,4′]bipiperidinyl-4′-carboxylic acid dimethylamide;
1′-{3-(3,4-dichlorophenyl)-4-[(2-1H-indol-3-yl-acetyl)-methyl-amino]-butyl}-[1,4′]bipiperidinyl-4′-carboxylic acid dimethylamide;
1′-{3-(3,4-dichlorophenyl)-4-[(1H-indole-5-carbonyl)-methyl-amino]-butyl}-[1,4′]bipiperidinyl-4′-carboxylic acid dimethylamide;
1′-{3-(3,4-dichlorophenyl)-4-[methyl-(pyrazine-2-carbonyl)-amino]-butyl}-[1,4′]bipiperidinyl-4′-carboxylic acid dimethylamide;
1′-{3-(3,4-dichlorophenyl)-4-[methyl-(pyridine-2-carbonyl)-amino]-butyl}-[1,4′]bipiperidinyl-4′-carboxylic acid dimethylamide;
1′-{3-(3,4-dichlorophenyl)-4-[methyl-(pyridine-4-carbonyl)-amino]-butyl}-[1,4′]bipiperidinyl-4′-carboxylic acid dimethylamide;
1′-{3-(3,4-dichlorophenyl)-4-[methyl-(4-oxo-4H-chromene-2-carbonyl)-amino]-butyl}-[1,4′]bipiperidinyl-4′-carboxylic acid dimethylamide;
1′-{3-(3,4-dichlorophenyl)-4-[methyl-(4-sulfamoyl-benzoyl)-amino]-butyl}-[1,4′]bipiperidinyl-4′-carboxylic acid dimethylamide;
1′-[4-[(4-chloro-3-sulfamoyl-benzoyl)-methyl-amino]-3-(3,4-dichlorophenyl)-butyl]-[1,4′]bipiperidinyl-4′-carboxylic acid dimethylamide;
1′-{3-(3,4-dichlorophenyl)-4-[(2-1H-imidazol-4-yl-acetyl)-methyl-amino]-butyl}-[1,4′]bipiperidinyl-4′-carboxylic acid dimethylamide;
1′-{3-(3,4-dichlorophenyl)-4-[methyl-(2-pyridin-2-yl-acetyl)-amino]-butyl}-[1,4′]bipiperidinyl-4′-carboxylic acid dimethylamide;
1′-{3-(3,4-dichlorophenyl)-4-[methyl-(2-pyridin-3-yl-acetyl)-amino]-butyl}-[1,4′]bipiperidinyl-4′-carboxylic acid dimethylamide;
1′-{3-(3,4-dichlorophenyl)-4-[methyl-(2-pyridin-4-yl-acetyl)-amino]-butyl}-[1,4′]bipiperidinyl-4′-carboxylic acid dimethylamide;
1′-[4-[(1-acetyl-piperidine-4-carbonyl)-methyl-amino]-3-(3,4-dichlorophenyl)-butyl]-[1,4′]bipiperidinyl-4′-carboxylic acid dimethylamide;
1′-{3-(3,4-dichlorophenyl)-4-[methyl-(tetrahydro-pyran-4-carbonyl)-amino]-butyl}-[1,4′]bipiperidinyl-4′-carboxylic acid dimethylamide;
(4-{[2-(3,4-dichlorophenyl)-4-(4′-dimethylcarbamoyl-[1,4′]bipiperidinyl-1′-yl)-butyl]-methyl-carbamoyl}-phenyl)-carbamic acid tert-butyl ester;
1′-{3-(3,4-dichlorophenyl)-4-[(3-{trifluoromethyl-methoxy}-benzoyl)-methyl-amino]-butyl}-[1,4′]bipiperidinyl-4′-carboxylic acid dimethylamide
1′-(3-(3,4-dichlorophenyl)-4-{[2-(2,4-di{trifluoromethyl}-phenyl)-acetyl]-methyl-amino}-butyl)-[1,4′]bipiperidinyl-4′-carboxylic acid dimethylamide;
1′-(3-(3,4-dichlorophenyl)-4-{[2-(2,6-dihydroxy-pyrimidin-4-yl)-acetyl]-methyl-amino}-butyl)-[1,4′]bipiperidinyl-4′-carboxylic acid dimethylamide;
4-{[2-(3,4-dichlorophenyl)-4-(4′-dimethylcarbamoyl-[1,4′]bipiperidinyl-1′-yl)-butyl]-methyl-carbamoyl}-piperidine-1-carboxylic acid tert-butyl ester;
1′-{3-(3,4-dichlorophenyl)-4-[(1H-imidazole-4-carbonyl)-methyl-amino]-butyl}-[1,4′]bipiperidinyl-4′-carboxylic acid dimethylamide;
(1-{[2-(3,4-dichlorophenyl)-4-(4′-dimethylcarbamoyl-[1,4′]bipiperidinyl-1′-yl)-butyl]-methyl-carbamoyl}-2-phenyl-ethyl)-carbamic acid tert-butyl ester;
[2-(3,4-dichlorophenyl)-4-(4′-dimethylcarbamoyl-[1,4′]bipiperidinyl-1′-yl)-butyl]-methyl-carbamic acid tert-butyl ester;
1′-{3-(3,4-dichlorophenyl)-4-[(furazan-3-carbonyl)-methyl-amino]-butyl}-[1,4′]bipiperidinyl-4′-carboxylic acid dimethylamide;
1′-{3-(3,4-dichlorophenyl)-4-[(2,2-difluoro-benzo[1,3]dioxole-5-carbonyl)-methyl-amino]-butyl}-[1,4′]bipiperidinyl-4′-carboxylic acid dimethylamide;
1′-{3-(3,4-dichlorophenyl)-4-[methyl-(1H-pyrrole-3-carbonyl)-amino]-butyl}-[1,4′]bipiperidinyl-4′-carboxylic acid dimethylamide;
1′-(3-(3,4-dichlorophenyl)-4-{[3-(4-fluorophenyl)-5-methyl-isoxazole-4-carbo-nyl]-methyl-amino}-butyl)-[1,4′]bipiperidinyl-4′-carboxylic acid dimethylamide;
1′-(3-(3,4-dichlorophenyl)-4-{[5-(4-methoxy-phenyl)-oxazole-4-carbonyl]-methyl-amino}-butyl)-[1,4′]bipiperidinyl-4′-carboxylic acid dimethylamide;
1′-{3-(3,4-dichlorophenyl)-4-[methyl-(5-methyl-1-phenyl-1H-[1,2,3]triazole-4-carbonyl)-amino]-butyl}-[1,4′]bipiperidinyl-4′-carboxylic acid dimethylamide;
1′-[4-[(benzofuran-5-carbonyl)-methyl-amino]-3-(3,4-dichlorophenyl)-butyl]-[1,4′]bipiperidinyl-4′-carboxylic acid dimethylamide;
1′-{3-(3,4-dichlorophenyl)-4-[methyl-(5-methyl-benzo[b]thiophene-2-carbonyl)-amino]-butyl}-[1,4′]bipiperidinyl-4′-carboxylic acid dimethylamide;
1′-[4-[(3,5-bis-trifluoromethyl-benzoyl)-methyl-amino]-3-(3,4-dichlorophenyl)-butyl]-[1,4′]bipiperidinyl-4′-carboxylic acid dimethylamide;
1′-[4-[(2-bromo-benzoyl)-methyl-amino]-3-(3,4-dichlorophenyl)-butyl]-[1,4′]bipiperidinyl-4′-carboxylic acid dimethylamide;
1′-{3-(3,4-dichlorophenyl)-4-[(2-fluoro-benzoyl)-methyl-amino]-butyl}-[1,4′]bipiperidinyl-4′-carboxylic acid dimethylamide;
1′-[3-(3,4-dichlorophenyl)-4-(methyl-pentafluorobenzoyl-amino)-butyl]-[1,4′]bipiperidinyl-4′-carboxylic acid dimethylamide;
1′-{3-(3,4-dichlorophenyl)-4-[(2,6-difluoro-benzoyl)-methyl-amino]-butyl}-[1,4′]bipiperidinyl-4′-carboxylic acid dimethylamide;
1′-[4-[(2,4-dichloro-benzoyl)-methyl-amino]-3-(3,4-dichlorophenyl)-butyl]-[1,4′]bipiperidinyl-4′-carboxylic acid dimethylamide;
1′-[4-[(2,6-dichloro-benzoyl)-methyl-amino]-3-(3,4-dichlorophenyl)-butyl]-[1,4′]bipiperidinyl-4′-carboxylic acid dimethylamide;
1′-{3-(3,4-dichlorophenyl)-4-[methyl-(2-trifluoromethyl-benzoyl)-amino]-butyl}-[1,4′]bipiperidinyl-4′-carboxylic acid dimethylamide;
1′-{3-(3,4-dichlorophenyl)-4-[methyl-(2-methyl-benzoyl)-amino]-butyl}-[1,4′]bipiperidinyl-4′-carboxylic acid dimethylamide;
1′-{3-(3,4-dichlorophenyl)-4-[(3-fluoro-benzoyl)-methyl-amino]-butyl}-[1,4′]bipiperidinyl-4′-carboxylic acid dimethylamide;
1′-[4-[(3-chloro-benzoyl)-methyl-amino]-3-(3,4-dichlorophenyl)-butyl]-[1,4′]bipiperidinyl-4′-carboxylic acid dimethylamide;
1′-[4-[(3,4-dichloro-benzoyl)-methyl-amino]-3-(3,4-dichlorophenyl)-butyl]-[1,4′]bipiperidinyl-4′-carboxylic acid dimethylamide;
1′-{3-(3,4-dichlorophenyl)-4-[(3-methoxy-benzoyl)-methyl-amino]-butyl}-[1,4′]bipiperidinyl-4′-carboxylic acid dimethylamide;
1′-{3-(3,4-dichlorophenyl)-4-[methyl-(3-trifluoromethyl-benzoyl)-amino]-butyl}-[1,4′]bipiperidinyl-4′-carboxylic acid dimethylamide;
1′-[4-[(4-chloro-benzoyl)-methyl-amino]-3-(3,4-dichlorophenyl)-butyl]-[1,4′]bipiperidinyl-4′-carboxylic acid dimethylamide;
1′-{3-(3,4-dichlorophenyl)-4-[(4-methoxy-benzoyl)-methyl-amino]-butyl}-[1,4′]bipiperidinyl-4′-carboxylic acid dimethylamide;
1′-{3-(3,4-dichlorophenyl)-4-[methyl-(4-trifluoromethyl-benzoyl)-amino]-butyl}-[1,4′]bipiperidinyl-4′-carboxylic acid dimethylamide;
1′-{3-(3,4-dichlorophenyl)-4-[(4-methyl-benzoyl)-methyl-amino]-butyl}-[1,4′]bipiperidinyl-4′-carboxylic acid dimethylamide;
1′-{3-(3,4-dichlorophenyl)-4-[(2,2-dimethyl-propionyl)-methyl-amino]-butyl}-[1,4′]bipiperidinyl-4′-carboxylic acid dimethylamide;
1′-[3-(3,4-dichlorophenyl)-4-(methyl-phenylacetyl-amino)-butyl]-[1,4′]bipiperidinyl-4′-carboxylic acid dimethylamide;
1′-{3-(3,4-dichlorophenyl)-4-[methyl-(2-phenyl-cyclopropanecarbonyl)-amino]-butyl}-[1,4′]bipiperidinyl-4′-carboxylic acid dimethylamide;
1′-[4-[(4-cyano-benzoyl)-methyl-amino]-3-(3,4-dichlorophenyl)-butyl]-[1,4′]bipiperidinyl-4′-carboxylic acid dimethylamide;
1′-{3-(3,4-dichlorophenyl)-4-[methyl-(naphthalene-1-carbonyl)-amino]-butyl}-[1,4′]bipiperidinyl-4′-carboxylic acid dimethylamide;
1′-[4-[(3-cyano-naphthalene-1-carbonyl)-methyl-amino]-3-(3,4-dichlorophenyl)-butyl]-[1,4′]bipiperidinyl-4′-carboxylic acid dimethylamide;
1′-[4-(benzoyl-methyl-amino)-3-phenyl-butyl]-[1,4′]bipiperidinyl-4′-carboxylic acid dimethylamide;
1′-[4-(benzoyl-methyl-amino)-3-(3,4-dichlorophenyl)-butyl]-[1,4′]bipiperidinyl-4′-carboxylic acid ethyl-methyl-amide;
N-{2-(3,4-dichlorophenyl)-4-[4-(1-dimethylcarbamoyl-cyclohexyl)-piperazin-1-yl]-butyl}-N-methyl-benzamide;
1′-[4-(benzoyl-methyl-amino)-3-(3,4-dichlorophenyl)-butyl]-[1,4′]bipiperidinyl-2-carboxylic acid dimethylamide;
1-[4-[(3-cyano-naphthalene-1-carbonyl)-methyl-amino]-3-(3,4-dichlorophenyl)-butyl]-4-pyrrolidin-1-yl-piperidine-4-carboxylic acid dimethylamide;
1-[4-(benzoyl-methyl-amino)-3-(3,4-dichlorophenyl)-butyl]-4-pyrrolidin-1-yl-piperidine-4-carboxylic acid dimethylamide;
N-[4-[4-(cyclopropylmethyl-propionyl-amino)-piperidin-1-yl]-2-(3,4-dichlorophenyl)-butyl]-N-methyl-benzamide;
N-{2-(3,4-dichlorophenyl)-4-[4-(isopropyl-propionyl-amino)-piperidin-1-yl]-butyl}-N-methyl-benzamide;
N-{2-(3,4-dichlorophenyl)-4-[4-(phenyl-propionyl-amino)-piperidin-1-yl]-butyl}-N-methyl-benzamide;
N-[4-[4-(butyl-propionyl-amino)-piperidin-1-yl]-2-(3,4-dichlorophenyl)-butyl]-N-methyl-benzamide;
N-[4-[4-(butyl-cyclopropanecarbonyl-amino)-piperidin-1-yl]-2-(3,4-dichlorophenyl)-butyl]-N-methyl-benzamide;
N-[4-[4-(butyl-cyclohexanecarbonyl-amino)-piperidin-1-yl]-2-(3,4-dichlorophenyl)-butyl]-N-methyl-benzamide;
N-[4-[4-(benzoyl-butyl-amino)-piperidin-1-yl]-2-(3,4-dichlorophenyl)-butyl]-N-methyl-benzamide;
N-(2-(3,4-dichlorophenyl)-4-{4-[(4-methoxy-butyl)-propionyl-amino]-piperidin-1-yl}-butyl)-N-methyl-benzamide;
N-[4-{4-[cyclopropanecarbonyl-(4-methoxy-butyl)-amino]-piperidin-1-yl}-2-(3,4-dichlorophenyl)-butyl]-N-methyl-benzamide;
N-[4-{4-[cyclohexanecarbonyl-(4-methoxy-butyl)-amino]-piperidin-1-yl}-2-(3,4-dichlorophenyl)-butyl]-N-methyl-benzamide;
N-[4-{4-[benzoyl-(4-methoxy-butyl)-amino]-piperidin-1-yl}-2-(3,4-dichlorophenyl)-butyl]-N-methyl-benzamide;
N-[4-{4-[cyclohexyl(propionyl)amino]piperidin-1-yl}-2-(3,4-dichlorophenyl)butyl]-N-methylbenzamide;
N-[4-{4-[cyclohexyl(cyclopropylcarbonyl)amino]piperidin-1-yl}-2-(3,4-dichlorophenyl)butyl]-N-methylbenzamide;
N-[4-{4-[cyclohexyl(cyclohexylcarbonyl)amino]piperidin-1-yl}-2-(3,4-dichlorophenyl)butyl]-N-methylbenzamide;
N-[4-{4-[benzoyl(cyclohexyl)amino]piperidin-1-yl}-2-(3,4-dichlorophenyl)butyl]-N-methylbenzamide;
N-[2-(3,4-dichlorophenyl)-4-{4-[(1-methylpiperidin-4-yl)(propionyl)amino]piperidin-1-yl}butyl]-N-methylbenzamide;
N-[4-{4-[(cyclopropylcarbonyl)(1-methylpiperidin-4-yl)amino]piperidin-1-yl}-2-(3,4-dichlorophenyl)butyl]-N-methylbenzamide;
N-[4-{4-[(cyclohexylcarbonyl)(1-methylpiperidin-4-yl)amino]piperidin-1-yl}-2-(3,4-dichlorophenyl)butyl]-N-methylbenzamide;
N-{1-[4-[benzoyl(methyl)amino]-3-(3,4-dichlorophenyl)butyl]piperidin-4-yl}-N-(1-methylpiperidin-4-yl)benzamide;
N-{2-(3,4-Dichlorophenyl)-4-[4′-(pyrrolidine-1-carbonyl)-[1,4′]bipiperidinyl-1′-yl]-butyl}-N-methyl-benzamide;
1-[4-[(3-cyano-naphthalene-1-carbonyl)-methyl-amino]-3-(3,4-dichlorophenyl)-butyl]-4-(2-oxo-pyrrolidin-1-yl)-piperidine-4-carboxylic acid dimethylamide;
3-cyano-naphthalene-1-carboxylic acid {2-(3,4-dichlorophenyl)-4-[4′-(piperidine-1-carbonyl)-[1,4′]bipiperidinyl-1′-yl]-butyl}-methyl-amide;
1′-[4-(benzoyl-methyl-amino)-3-(3,4-dichlorophenyl)-butyl]-[1,4′]bipiperidinyl-4′-carboxylic acid di-propylamide;
1-[4-(benzoyl-methyl-amino)-3-(3,4-dichlorophenyl)-butyl]-4-morpholin-4-yl-piperidine-4-carboxylic acid dimethylamide;
1-[4-(benzoyl-methyl-amino)-3-(3,4-dichlorophenyl)-butyl]-4-pyrrolidin-1-yl-piperidine-4-carboxylic acid isopropyl-methyl-amide;
N-{2-(3,4-dichlorophenyl)-4-[4-(piperidine-1-carbonyl)-4-pyrrolidin-1-yl-piperidin-1-yl]-butyl}-N-methyl-benzamide;
1-[4-(benzoyl-methyl-amino)-3-(3,4-dichlorophenyl)-butyl]-4-pyrrolidin-1-yl-piperidine-4-carboxylic acid diethylamide;
N-{2-(3,4-dichlorophenyl)-4-[4-(morpholine-4-carbonyl)-4-pyrrolidin-1-yl-piperidin-1-yl]-butyl}-N-methyl-benzamide; and
physiologically compatible salts of these compounds.

12. A compound according to claim 11, selected from the group consisting of:
1′-[4-(benzoyl-methyl-amino)-3-(3,4-dichlorophenyl)-butyl]-[1,4′]bipiperidinyl-4′-carboxylic acid dimethylamide;
1′-[4-(benzoyl-methyl-amino)-3-(3,4-dichlorophenyl)-butyl]-[1,4′]bipiperidinyl-4′-carboxylic acid methylamide;
1′-[4-(cyclohexanecarbonyl-methyl-amino)-3-(3,4-dichlorophenyl)-butyl]-[1,4′]bipiperidinyl-4′-carboxylic acid dimethylamide;
1′-{3-(3,4-dichlorophenyl)-4-[(4-fluoro-benzoyl)-methyl-amino]-butyl}-[1,4′]bipiperidinyl-4′-carboxylic acid dimethylamide;
acetic acid 4-{[2-(3,4-dichlorophenyl)-4-(4′-dimethylcarbamoyl-[1,4′]bi-piperidinyl-1′-yl)-butyl]-methyl-carbamoyl}-phenyl ester;
1′-{3-(3,4-dichlorophenyl)-4-[(4-hydroxy-benzoyl)-methyl-amino]-butyl}-[1,4′]bi-piperidinyl-4′-carboxylic acid dimethylamide;
acetic acid 2-{[2-(3,4-dichlorophenyl)-4-(4′-dimethylcarbamoyl-[1,4′]bi-piperidinyl-1′-yl)-butyl]-methyl-carbamoyl}-phenyl ester;
1′-[4-[(3-chloro-4-fluoro-benzoyl)-methyl-amino]-3-(3,4-dichlorophenyl)-butyl]-[1,4′]bipiperidinyl-4′-carboxylic acid dimethylamide;
1′{-3-(3,4-dichlorophenyl)-4-[(3,5-difluoro-benzoyl)-methyl-amino]-butyl}-[1,4′]bipiperidinyl-4′-carboxylic acid dimethylamide;
1′-[4-[(5-chloro-2-fluoro-benzoyl)-methyl-amino]-3-(3,4-dichlorophenyl)-butyl]-[1,4′]bipiperidinyl-4′-carboxylic acid dimethylamide;
1′-{3-(3,4-dichlorophenyl)-4-[methyl-(naphthalene-1-carbonyl-3-cyano)-amino]-butyl}-[1,4′]bipiperidinyl-4′-carboxylic acid dimethylamide;
1′-{3-(3,4-dichlorophenyl)-4-[(2-hydroxy-benzoyl)-methyl-amino]-butyl}-[1,4′]bipiperidinyl-4′-carboxylic acid dimethylamide;
1′-{3-(3,4-dichlorophenyl)-4-[(2,4-difluoro-benzoyl)-methyl-amino]-butyl}-[1,4′]bipiperidinyl-4′-carboxylic acid dimethylamide;
1′-{3-(3,4-dichlorophenyl)-4-[(3,4-difluoro-benzoyl)-methyl-amino]-butyl}-[1,4′]bipiperidinyl-4′-carboxylic acid dimethylamide;
1′-{3-(3,4-dichlorophenyl)-4-[(2,5-difluoro-benzoyl)-methyl-amino]-butyl}-[1,4′]bipiperidinyl-4′-carboxylic acid dimethylamide;
1′-{3-(3,4-dichlorophenyl)-4-[methyl-(2,3,4-trifluoro-benzoyl)-amino]-butyl}-[1,4′]bipiperidinyl-4′-carboxylic acid dimethylamide;
1′-[4-[(6-chloro-pyridine-3-carbonyl)-methyl-amino]-3-(3,4-dichlorophenyl)-butyl]-[1,4′]bipiperidinyl-4′-carboxylic acid dimethylamide;
1′-{3-(3,4-dichlorophenyl)-4-[methyl-(pyridine-3-carbonyl)-amino]-butyl}-[1,4′]bipiperidinyl-4′-carboxylic acid dimethylamide;
1′-[4-(cyclopentanecarbonyl-methyl-amino)-3-(3,4-dichlorophenyl)-butyl]-[1,4′]bipiperidinyl-4′-carboxylic acid dimethylamide;
1′-{3-(3,4-dichlorophenyl)-4-[methyl-(2-methylamino-benzoyl)-amino]-butyl}-[1,4′]bipiperidinyl-4′-carboxylic acid dimethylamide;
N-[2-(3,4-dichlorophenyl)-4-(4′-dimethylcarbamoyl-[1,4′]bipiperidinyl-1′-yl)-butyl]-N-methyl-phthalamic acid;
1′-{3-(3,4-dichlorophenyl)-4-[(4-methoxy-benzoyl)-methyl-amino]-butyl}-[1,4′]bipiperidinyl-4′-carboxylic acid dimethylamide;
1′-(3-(3,4-dichlorophenyl)-4-{[3-(4-hydroxy-phenyl)-propionyl]-methyl-amino}-butyl)-[1,4′]bipiperidinyl-4′-carboxylic acid dimethylamide;
1′-{3-(3,4-dichlorophenyl)-4-[methyl-(1-methyl-1H-pyrrole-2-carbonyl)-amino]-butyl}-[1,4′]bipiperidinyl-4′-carboxylic acid dimethylamide;
1′-{3-(3,4-dichlorophenyl)-4-[(furan-2-carbonyl)-methyl-amino]-butyl}-[1,4′]bipiperidinyl-4′-carboxylic acid dimethylamide;
1′-{3-(3,4-dichlorophenyl)-4-[methyl-(naphthalene-2-carbonyl)-amino]-butyl}-[1,4′]bipiperidinyl-4′-carboxylic acid dimethylamide;
1′-{3-(3,4-dichlorophenyl)-4-[methyl-(1H-pyrrole-2-carbonyl)-amino]-butyl}-[1,4′]bipiperidinyl-4′-carboxylic acid dimethylamide;
1′-{3-(3,4-dichlorophenyl)-4-[(furan-2-carbonyl)-methyl-amino]-butyl}-[1,4′]bipiperidinyl-4′-carboxylic acid dimethylamide;
1′-{3-(3,4-dichlorophenyl)-4-[methyl-(thiophene-2-carbonyl)-amino]-butyl}-[1,4′]bipiperidinyl-4′-carboxylic acid dimethylamide;
1′-{3-(3,4-dichlorophenyl)-4-[methyl-(thiophene-3-carbonyl)-amino]-butyl}-[1,4′]bipiperidinyl-4′-carboxylic acid dimethylamide;
1′-{3-(3,4-dichlorophenyl)-4-[(1H-indole-5-carbonyl)-methyl-amino]-butyl}-[1,4′]bipiperidinyl-4′-carboxylic acid dimethylamide;
1′-{3-(3,4-dichlorophenyl)-4-[methyl-(pyridine-2-carbonyl)-amino]-butyl}-[1,4′]bipiperidinyl-4′-carboxylic acid dimethylamide;
1′-{3-(3,4-dichlorophenyl)-4-[methyl-(pyridine-4-carbonyl)-amino]-butyl}-[1,4′]bipiperidinyl-4′-carboxylic acid dimethylamide;
1′-{3-(3,4-dichlorophenyl)-4-[methyl-(4-oxo-4H-chromene-2-carbonyl)-amino]-butyl}-[1,4′]bipiperidinyl-4′-carboxylic acid dimethylamide;
1′-{3-(3,4-dichlorophenyl)-4-[methyl-(4-sulfamoyl-benzoyl)-amino]-butyl}-[1,4′]bipiperidinyl-4′-carboxylic acid dimethylamide;
1′-[4-[(4-chloro-3-sulfamoyl-benzoyl)-methyl-amino]-3-(3,4-dichlorophenyl)-butyl]-[1,4′]bipiperidinyl-4′-carboxylic acid dimethylamide;
1′-{3-(3,4-dichlorophenyl)-4-[methyl-(2-pyridin-2-yl-acetyl)-amino]-butyl}-[1,4′]bipiperidinyl-4′-carboxylic acid dimethylamide;
1′-{3-(3,4-dichlorophenyl)-4-[methyl-(2-pyridin-3-yl-acetyl)-amino]-butyl}-[1,4′]bipiperidinyl-4′-carboxylic acid dimethylamide;
1′-{3-(3,4-dichlorophenyl)-4-[methyl-(2-pyridin-4-yl-acetyl)-amino]-butyl}-[1,4′]bipiperidinyl-4′-carboxylic acid dimethylamide;
1′-{3-(3,4-dichlorophenyl)-4-[methyl-(tetrahydro-pyran-4-carbonyl)-amino]-butyl}-[1,4′]bipiperidinyl-4′-carboxylic acid dimethylamide;
(4-{[2-(3,4-dichlorophenyl)-4-(4′-dimethylcarbamoyl-[1,4′]bipiperidinyl-1′-yl)-butyl]-methyl-carbamoyl}-phenyl)-carbamic acid tert-butyl ester;
1′-{3-(3,4-dichlorophenyl)-4-[(3-{trifluoromethyl-methoxy}-benzoyl)-methyl-amino]-butyl}-[1,4′]bipiperidinyl-4′-carboxylic acid dimethylamide
1′-(3-(3,4-dichlorophenyl)-4-{[2-(2,4-di{trifluoromethyl}-phenyl)-acetyl]-methyl-amino}-butyl)-[1,4′]bipiperidinyl-4′-carboxylic acid dimethylamide;
1′-{3-(3,4-dichlorophenyl)-4-[(2,2-difluoro-benzo[1,3]dioxole-5-carbonyl)-methyl-amino]-butyl}-[1,4′]bipiperidinyl-4′-carboxylic acid dimethylamide;
1′-{3-(3,4-dichlorophenyl)-4-[methyl-(5-methyl-1-phenyl-1H-[1,2,3]triazole-4-carbonyl)-amino]-butyl}-[1,4′]bipiperidinyl-4′-carboxylic acid dimethylamide;
1′-[4-[(benzofuran-5-carbonyl)-methyl-amino]-3-(3,4-dichlorophenyl)-butyl]-[1,4′]bipiperidinyl-4′-carboxylic acid dimethylamide;
1′-[4-[(2-bromo-benzoyl)-methyl-amino]-3-(3,4-dichlorophenyl)-butyl]-[1,4′]bipiperidinyl-4′-carboxylic acid dimethylamide;
1′-{3-(3,4-dichlorophenyl)-4-[(2-fluoro-benzoyl)-methyl-amino]-butyl}-[1,4′]bipiperidinyl-4′-carboxylic acid dimethylamide;
1′-{3-(3,4-dichlorophenyl)-4-[(2,6-difluoro-benzoyl)-methyl-amino]-butyl}-[1,4′]bipiperidinyl-4′-carboxylic acid dimethylamide;
1′-[4-[(2,4-dichloro-benzoyl)-methyl-amino]-3-(3,4-dichlorophenyl)-butyl]-[1,4′]bipiperidinyl-4′-carboxylic acid dimethylamide;
1′{-3-(3,4-dichlorophenyl)-4-[methyl-(2-trifluoromethyl-benzoyl)-amino]-butyl}-[1,4′]bipiperidinyl-4′-carboxylic acid dimethylamide;
1′-{3-(3,4-dichlorophenyl)-4-[methyl-(2-methyl-benzoyl)-amino]-butyl}-[1,4′]bipiperidinyl-4′-carboxylic acid dimethylamide;
1′-{3-(3,4-dichlorophenyl)-4-[(3-fluoro-benzoyl)-methyl-amino]-butyl}-[1,4′]bipiperidinyl-4′-carboxylic acid dimethylamide;
1′-[4-[(3-chloro-benzoyl)-methyl-amino]-3-(3,4-dichlorophenyl)-butyl]-[1,4′]bipiperidinyl-4′-carboxylic acid dimethylamide;
1′-{3-(3,4-dichlorophenyl)-4-[(3-methoxy-benzoyl)-methyl-amino]-butyl}-[1,4′]bipiperidinyl-4′-carboxylic acid dimethylamide;
1′-{3-(3,4-dichlorophenyl)-4-[methyl-(3-trifluoromethyl-benzoyl)-amino]-butyl}-[1,4′]bipiperidinyl-4′-carboxylic acid dimethylamide;
1′-{3-(3,4-dichlorophenyl)-4-[(4-methyl-benzoyl)-methyl-amino]-butyl}-[1,4′]bipiperidinyl-4′-carboxylic acid dimethylamide;
1′-[4-[(4-cyano-benzoyl)-methyl-amino]-3-(3,4-dichlorophenyl)-butyl]-[1,4′]bipiperidinyl-4′-carboxylic acid dimethylamide;
1′-[4-[(3-cyano-naphthalene-1-carbonyl)-methyl-amino]-3-(3,4-dichlorophenyl)-butyl]-[1,4′]bipiperidinyl-4′-carboxylic acid dimethylamide;
1′-[4-(benzoyl-methyl-amino)-3-phenyl-butyl]-[1,4′]bipiperidinyl-4′-carboxylic acid dimethylamide;
1′-[4-(benzoyl-methyl-amino)-3-(3,4-dichlorophenyl)-butyl]-[1,4′]bipiperidinyl-4′-carboxylic acid ethyl-methyl-amide;
N-{2-(3,4-dichlorophenyl)-4-[4-(1-dimethylcarbamoyl-cyclohexyl)-piperazin-1-yl]-butyl}-N-methyl-benzamide;
1′-[4-(benzoyl-methyl-amino)-3-(3,4-dichlorophenyl)-butyl]-[1,4′]bipiperidinyl-2-carboxylic acid dimethylamide;
1-[4-[(3-cyano-naphthalene-1-carbonyl)-methyl-amino]-3-(3,4-dichlorophenyl)-butyl]-4-pyrrolidin-1-yl-piperidine-4-carboxylic acid dimethylamide;
1-[4-(benzoyl-methyl-amino)-3-(3,4-dichlorophenyl)-butyl]-4-pyrrolidin-1-yl-piperidine-4-carboxylic acid dimethylamide;
and physiologically compatible acid addition salts of these compounds.

13. A compound according to claim 11, selected from the group consisting of:
1′-[4-(benzoyl-methyl-amino)-3-(3,4-dichlorophenyl)-butyl]-[1,4′]bipiperidinyl-4′-carboxylic acid dimethylamide;
1′-[4-(cyclohexanecarbonyl-methyl-amino)-3-(3,4-dichlorophenyl)-butyl]-[1,4′]bipiperidinyl-4′-carboxylic acid dimethylamide;
1′-{3-(3,4-dichlorophenyl)-4-[(4-fluoro-benzoyl)-methyl-amino]-butyl}-[1,4′]bipiperidinyl-4′-carboxylic acid dimethylamide;
acetic acid 4-{[2-(3,4-dichlorophenyl)-4-(4′-dimethylcarbamoyl-[1,4′]bi-piperidinyl-1′-yl)-butyl]-methyl-carbamoyl}-phenyl ester;
1′-{3-(3,4-dichlorophenyl)-4-[(4-hydroxy-benzoyl)-methyl-amino]-butyl}-[1,4′]bi-piperidinyl-4′-carboxylic acid dimethylamide;
acetic acid 2-{[2-(3,4-dichlorophenyl)-4-(4′-dimethylcarbamoyl-[1,4′]bi-piperidinyl-1′-yl)-butyl]-methyl-carbamoyl}-phenyl ester;
1′-[4-[(3-chloro-4-fluoro-benzoyl)-methyl-amino]-3-(3,4-dichlorophenyl)-butyl]-[1,4′]bipiperidinyl-4′-carboxylic acid dimethylamide;
1′-{3-(3,4-dichlorophenyl)-4-[(3,5-difluoro-benzoyl)-methyl-amino]-butyl}-[1,4′]bipiperidinyl-4′-carboxylic acid dimethylamide;
1′-[4-[(5-chloro-2-fluoro-benzoyl)-methyl-amino]-3-(3,4-dichlorophenyl)-butyl]-[1,4′]bipiperidinyl-4′-carboxylic acid dimethylamide;
1′-{3-(3,4-dichlorophenyl)-4-[methyl-(naphthalene-1-carbonyl-3-cyano)-amino]-butyl}-[1,4′]bipiperidinyl-4′-carboxylic acid dimethylamide;
1′-{3-(3,4-dichlorophenyl)-4-[(2-hydroxy-benzoyl)-methyl-amino]-butyl}-[1,4′]bipiperidinyl-4′-carboxylic acid dimethylamide;
1′-{3-(3,4-dichlorophenyl)-4-[(2,4-difluoro-benzoyl)-methyl-amino]-butyl}-[1,4′]bipiperidinyl-4′-carboxylic acid dimethylamide;
1′-{3-(3,4-dichlorophenyl)-4-[(3,4-difluoro-benzoyl)-methyl-amino]-butyl}-[1,4′]bipiperidinyl-4′-carboxylic acid dimethylamide;
1′-{3-(3,4-dichlorophenyl)-4-[(2,5-difluoro-benzoyl)-methyl-amino]-butyl}-[1,4′]bipiperidinyl-4′-carboxylic acid dimethylamide;
1′-[4-[(6-chloro-pyridine-3-carbonyl)-methyl-amino]-3-(3,4-dichlorophenyl)-butyl]-[1,4′]bipiperidinyl-4′-carboxylic acid dimethylamide;
1′-[4-(cyclopentanecarbonyl-methyl-amino)-3-(3,4-dichlorophenyl)-butyl]-[1,4′]bipiperidinyl-4′-carboxylic acid dimethylamide;
1′-{3-(3,4-dichlorophenyl)-4-[(furan-2-carbonyl)-methyl-amino]-butyl}-[1,4′]bipiperidinyl-4′-carboxylic acid dimethylamide;
1′-{3-(3,4-dichlorophenyl)-4-[methyl-(pyridine-2-carbonyl)-amino]-butyl}-[1,4′]bipiperidinyl-4′-carboxylic acid dimethylamide;
1′-{3-(3,4-dichlorophenyl)-4-[methyl-(2-pyridin-2-yl-acetyl)-amino]-butyl}-[1,4′]bipiperidinyl-4′-carboxylic acid dimethylamide;
1′{-3-(3,4-dichlorophenyl)-4-[methyl-(2-pyridin-3-yl-acetyl)-amino]-butyl}-[1,4′]bipiperidinyl-4′-carboxylic acid dimethylamide;
1′-{3-(3,4-dichlorophenyl)-4-[(3-{trifluoromethyl-methoxy}-benzoyl)-methyl-amino]-butyl}-[1,4′]bipiperidinyl-4′-carboxylic acid dimethylamide;
1′-{3-(3,4-dichlorophenyl)-4-[methyl-(5-methyl-1-phenyl-1H-[1,2,3]triazole-4-carbonyl)-amino]-butyl}-[1,4′]bipiperidinyl-4′-carboxylic acid dimethylamide;
1′-[4-[(benzofuran-5-carbonyl)-methyl-amino]-3-(3,4-dichlorophenyl)-butyl]-[1,4′]bipiperidinyl-4′-carboxylic acid dimethylamide;
1′-[4-[(3-cyano-naphthalene-1-carbonyl)-methyl-amino]-3-(3,4-dichlorophenyl)-butyl]-[1,4′]bipiperidinyl-4′-carboxylic acid dimethylamide;
1-[4-[(3-cyano-naphthalene-1-carbonyl)-methyl-amino]-3-(3,4-dichlorophenyl)-butyl]-4-pyrrolidin-1-yl-piperidine-4-carboxylic acid dimethylamide; and
physiologically compatible salts of these compounds.

14. A pharmaceutical composition comprising:
(a) a pharmacologically effective quantity of a compound of Formula I, 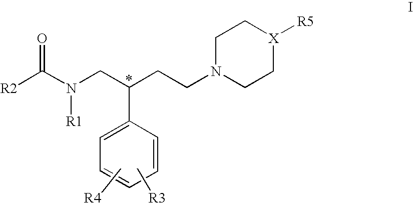
wherein
R1 is selected from the group consisting of alkyl and cycloalkyl;
R2 is selected from the group consisting of alkyl, cycloalkyl, aryl, alkylenearyl, alkenylenearyl, heteroaryl, and heterocyclic rings;
R3 and R4 are independently selected from the group consisting of hydrogen, halogen, hydroxyl, cyano, and carboxyalkyl;
X is selected from the group consisting of CR6 and nitrogen;
R5 is selected from the group consisting of alkyl optionally substituted with (CO)ₘNR9R10, cycloalkyl optionally substituted with (CO)ₘNR9R10, and NR7R8;
R6 is selected from the group consisting of hydrogen, alkyl, cycloalkyl, and (CO)ₘNR9R10;
R7 and R8 are independently selected from the group consisting of alkyl, cycloalkyl, aryl, alkylenearyl, alkyleneoxyalkyl, COalkyl, COaryl, or wherein R7 and R8 form together a 5- to 7-membered ring optionally containing an additional heteroatom, wherein such ring may be substituted by CONR9R10, and wherein in a 6-membered ring, none of the ring atoms is replaced by carbonyl;
R9 and R10 are independently selected from the group consisting of hydrogen, alkyl, cycloalkyl, aryl, alkylenearyl, alkyleneoxyalkyl, or wherein R9 and R10 form together a 5- to 7-membered ring optionally containing an additional heteroatom;
m is 0 or 1;
or a physiologically acceptable salt thereof; and
(b) at least one pharmaceutically acceptable auxiliary or carrier.

15. A method of preparing a compound corresponding to formula I: 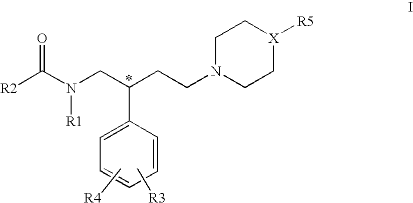
wherein
R1 is selected from the group consisting of alkyl and cycloalkyl;
R2 is selected from the group consisting of alkyl, cycloalkyl, aryl, alkylenearyl, alkenylenearyl, heteroaryl, and heterocyclic rings;
R3 and R4 are independently selected from the group consisting of hydrogen, halogen, hydroxyl, cyano, and carboxyalkyl;
X is selected from the group consisting of CR6 and nitrogen;
R5 is selected from the group consisting of alkyl optionally substituted with (CO)ₘNR9R10, cycloalkyl optionally substituted with (CO)ₘNR9R10, and NR7R8;
R6 is selected from the group consisting of hydrogen, alkyl, cycloalkyl, and (CO)ₘNR9R10;
R7 and R8 are independently selected from the group consisting of alkyl, cycloalkyl, aryl, alkylenearyl, alkyleneoxyalkyl, COalkyl, COaryl, or wherein R7 and R8 form together a 5- to 7-membered ring optionally containing an additional heteroatom, wherein such ring may be substituted by CONR9R10, and wherein in a 6-membered ring, none of the ring atoms is replaced by carbonyl;
R9 and R10 are independently selected from the group consisting of hydrogen, alkyl, cycloalkyl, aryl, alkylenearyl, alkyleneoxyalkyl, or wherein R9 and R10 form together a 5- to 7-membered ring optionally containing an additional heteroatom;
m is 0 or 1; or a physiologically acceptable salt thereof;
said method comprising:
(a) reacting a compound of formula II: 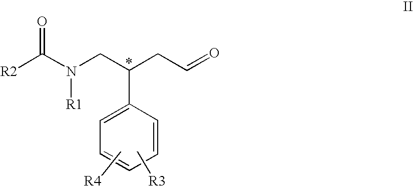
with a compound of formula III: 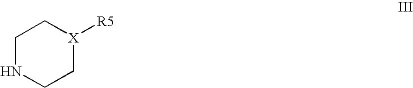
to yield a compound of formula I, and
optionally converting the compound of formula I to a physiologically compatible salt; or
(b) reacting a compound of formula III: 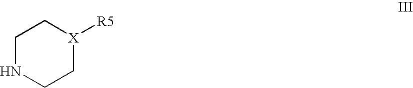
with a compound of formula IV: 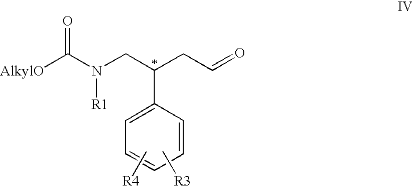
to yield a compound of formula V: 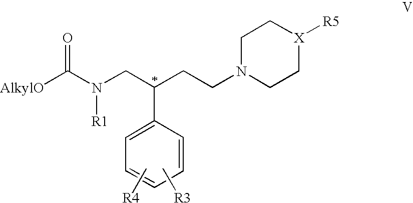
hydrolyzing the compound of formula V in an acidic medium to yield a compound of formula VI: 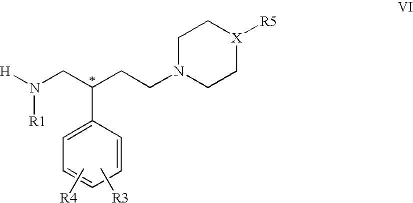
reacting the compound of formula VI with a compound of formula VII: 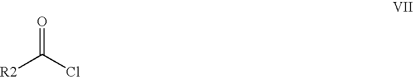
to yield a compound of formula I, and
optionally converting the compound of formula I to a physiologically compatible salt; or
(c) reacting a a compound of formula X: 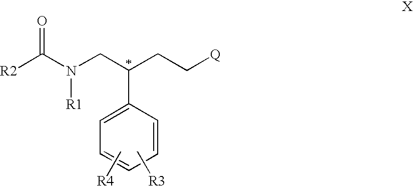
wherein Q is selected from the group consisting of halogen, and methylsulfonyl;
with a compound of formula III 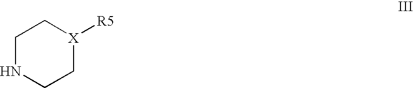
to yield a compound of formula I, and
optionally converting the compound of formula I to a physiologically compatible salt.

16. A method of treating or inhibiting a pathological state mediated by at least one enzyme or receptor selected from the group consisting of neurokinin A, a NK2 receptor, neurokinin B, and a NK3 receptor in a patient, said method comprising administering to said patient a therapeutically effective amount of a compound according to claim 1.

17. A method of treating or inhibiting a pathological state mediated by at least one enzyme or receptor selected from the group consisting of substance P, a NK1 receptor, neurokinin A, a NK2 receptor, neurokinin B, and a NK3 receptor in a patient, said method comprising administering to said patient a therapeutically effective amount of a compound according to claim 1, wherein R2 is a cyano-substituted naphthalene ring system.

18. A method of treating or inhibiting a pathological state of the respiratory, gastrointestinal, urinary, immune, cardiovascular or central nervous system or pain, migraine, inflammation, nausea, vomiting, or a skin disease in a patient in need thereof, said method comprising administering to said patient a therapeutically effective amount of a compound according to claim 1.

19. A method of treating or inhibiting a respiratory disease selected from the group consisting of asthma, chronic obstructive pulmonary disease, chronic obstructive bronchitis, bronchitis, cough, and rhinitis; or a skin disease selected from the group consisting of inflammatory skin reactions, allergic skin reactions, and psoriasis; or an arthropathic disease selected from the group consisting of arthritis, vasculitides and systemic lupus erythematosus; or a functional or inflammatory disorder of the gastrointestinal tract selected from the group consisting of pseudomembranous colitis, gastritis, acute and chronic pancreatitis, ulcerative colitis, Crohn's disease and diarrhea; or a bladder disease selected from the group consisting of cystitis and interstitial cystitis; or a cardiovascular disease; or a cancer selected from the group consisting of melanomas, gliomas, small-cell and large-cell lung cancers; or a disease of the immune system, bipolar disorders; migraine; pain, anxiety, depression, a cognitive disorder, a stress-related somatic disorder; or a psychosis selected from the group consisting of schizophrenia, mania, schizoaffective disorder and panic disorders in a patient, said method comprising administering to said patient a therapeutically effective amount of a compound according to claim 1.

20. A method of treating or inhibiting a pathological state mediated by at least one enzyme or receptor selected from the group consisting of neurokinin A, NK2 receptors, neurokinin B, and NK3 receptors, in a human or other mammal in need thereof, said method comprising administering to said human or other mammal a therapeutically effective amount of a compound of Formula I: 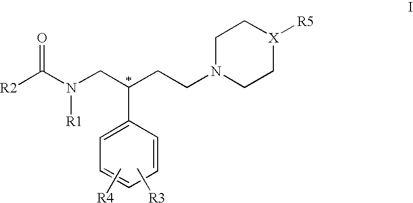
wherein
R1 is selected from the group consisting of alkyl and cycloalkyl;
R2 is selected from the group consisting of alkyl, cycloalkyl, aryl, alkylenearyl, alkenylenearyl, heteroaryl, and heterocyclic ring;
R3 and R4 are independently selected from the group consisting of hydrogen, halogen, hydroxyl, cyano, and carboxyalkyl;
X is selected from the group consisting of CR6 and nitrogen;
R5 is selected from the group consisting of alkyl optionally substituted with (CO)ₘNR9R10, cycloalkyl optionally substituted with (CO)ₘNR9R10, and NR7R8;
R6 is selected from the group consisting of hydrogen, alkyl, cycloalkyl, and (CO)ₘNR9R10;
R7 and R8 are independently selected from the group consisting of alkyl, cycloalkyl, aryl, alkylenearyl, alkyleneoxyalkyl, COalkyl, COaryl, or wherein R7 and R8 form together a 5- to 7-membered ring optionally containing an additional heteroatom, wherein such ring may be substituted by CONR9R10, and wherein in a 6-membered ring, none of the ring atoms is replaced by carbonyl;
R9 and R10 are independently selected from the group consisting of hydrogen, alkyl, cycloalkyl, aryl, alkylenearyl, alkyleneoxyalkyl, or wherein R9 and R10 form together a 5- to 7-membered ring optionally containing an additional heteroatom;
m is 0 or 1; or
a physiologically acceptable salt thereof.

21. A method of treating or inhibiting a pathological state mediated by at least one enzyme or receptor selected from the group consisting of substance P, NK1 receptors, neurokinin A, NK2 receptors, neurokinin B and NK3 receptors, in a human or other mammal in need thereof, said method comprising administering to said human or other mammal a therapeutically effective amount of a compound of Formula I, 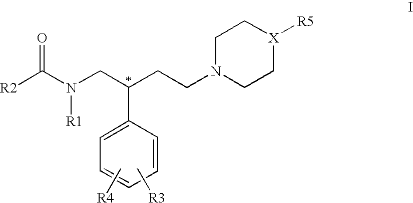
wherein
R1 is selected from the group consisting of alkyl and cycloalkyl;
R2 is a cyano-substituted naphthalene ring system;
R3 and R4 are independently selected from the group consisting of hydrogen, halogen, hydroxyl, cyano, and carboxyalkyl;
X is selected from the group consisting of CR6 and nitrogen;
R5 is selected from the group consisting of alkyl optionally substituted with (CO)ₘNR9R10, cycloalkyl optionally substituted with (CO)ₘNR9R10, and NR7R8;
R6 is selected from the group consisting of hydrogen, alkyl, cycloalkyl, and (CO)ₘNR9R10;
R7 and R8 are independently selected from the group consisting of alkyl, cycloalkyl, aryl, alkylenearyl, alkyleneoxyalkyl, COalkyl, COaryl, or wherein R7 and R8 form together a 5- to 7-membered ring optionally containing an additional heteroatom, wherein such ring may be substituted by CONR9R10, and wherein in a 6-membered ring, none of the ring atoms is replaced by carbonyl;
R9 and R10 are independently selected from the group consisting of hydrogen, alkyl, cycloalkyl, aryl, alkylenearyl, alkyleneoxyalkyl, or wherein R9 and R10 form together a 5- to 7-membered ring optionally containing an additional heteroatom;
m is 0 or 1; or
a physiologically compatible salt thereof.

22. A method according to claim 19, wherein a pathological state of the respiratory, gastrointestinal, urinary, immune, cardiovascular or central nervous system, or pain, migraine, inflammation, nausea, vomiting, or a skin disease is treated or inhibited.

23. A method according to claim 19, wherein a respiratory disease selected from the group consisting of asthma, chronic obstructive pulmonary disease, chronic obstructive bronchitis, bronchitis, cough, and rhinitis; or a skin disease selected from the group consisting of inflammatory skin reactions, allergic skin reactions, and psoriasis; or an arthropathic disease selected from the group consisting of arthritis, vasculitides and systemic lupus erythematosus; or a functional or inflammatory disorder of the gastrointestinal tract selected from the group consisting of pseudomembranous colitis, gastritis, acute and chronic pancreatitis, ulcerative colitis, Crohn's disease and diarrhea; or a bladder disease selected from the group consisting of cystitis and interstitial cystitis; or a cardiovascular disease; or a cancer selected from the group consisting of melanomas, gliomas, small-cell and large-cell lung cancers; or a disease of the immune system, bipolar disorder; migraine; anxiety, depression, cognitive disorder, stress-related somatic disorder; or a psychosis selected from the group consisting of schizophrenia, mania, schizoaffective disorder and panic disorders is treated or inhibited.
